(19) United States
(12) Patent Application Publication
(10) Pub. No.: US 2024/0218363 A1
(43) Pub. Date: Jul. 4, 2024

(54) OPTIMIZED 2'- MODIFIED RIBOSE DERIVATIVES AND METHODS OF USE

(71) Applicant: Sanegene Bio USA Inc., Woburn, MA (US)

(72) Inventors: Weimin WANG, Winchester, MA (US); Xiaochuan CAI, Woburn, MA (US); Shiyu WANG, Belmont, MA (US)

(21) Appl. No.: 18/350,256

(22) Filed: Jul. 11, 2023

Related U.S. Application Data
(60) Provisional application No. 63/359,969, filed on Jul. 11, 2022.

(51) Int. Cl. C12N 15/113

(52) U.S. Cl. CPC C12N 15/113; C12N 2310/11; C12N 2310/31; C12N 2310/321; C12N 2310/322; C12N 2310/332; C12N 2310/351

(57) ABSTRACT

The present disclosure provides optimized isolated oligonucleotides comprising specific combination of 2′-ribose derivatives or substitutions, to enhance stability and potency of the same for delivery and use as therapeutic agents.

### RELATED APPLICATIONS

This application claims priority to, and the benefit of, U.S. Provisional Application 63/359,969, filed Jul. 11, 2022, the entire contents of which are incorporated herein by reference.

### BACKGROUND

Small interfering RNAs (siRNAs) can efficiently trigger RNAi silencing of specific genes, but their therapeutic potential still faces challenges involving potency, safety, and delivery. Fully chemically modified siRNA molecules with a combination of ribose modification (e.g., 2′-O-methyl and 2′-deoxy-2′-fluoro), backbone modification (e.g., phosphorothioate), and 5′-phosphate modifications (e.g., 5′-E-vinylphosphonate) have been widely investigated and shown to significantly improve the metabolic stability and RNAi activity, leading to the enhanced potency and duration in vivo.

Among these chemical modifications, substitution of the 2′-OH group of ribose with 2′-O-methyl (2′-OMe), 2′-deoxy-2′-fluoro (2′-F) and other 2′-O-alkyl groups (e.g., 2′-O-methoxyethyl) have been demonstrated to enhance the metabolic stability and RNAi activity of siRNAs. More specifically, 2′-O-methyl modifications are sterically more hindered than 2′-deoxy-2′-fluoro and have shown stronger metabolic stability against endogenous nucleases. Also, it may be desirable to mitigate the concern about the tolerability of incorporating unnatural 2′-fluoro modification into siRNAs, although the 2′-fluoro containing siRNAs have been thoroughly examined in preclinical studies and well tolerated in clinical trials. Therefore, an increase of the 2′-O-methyl content with a concomitant decrease of the 2′-fluoro content in siRNAs is preferred in terms of improvement of metabolic stability and tolerability. However, the sterically more hindered 2′-O-methyl group can cause steric clashes with Argonaute RISC Catalytic Component 2 (Ago2) residues, thereby preventing guide strand loading into the RISC and substantially reducing RNAi activity if it is not applied judiciously. Thus, there continues to be a need for optimal chemical modifications that comprehensively enhance the metabolic stability, tolerability, and RNAi activity of siRNAs. The present disclosure addresses this need.

### SUMMARY

Provided herein is an isolated oligonucleotide, comprising: (a) a sense strand comprising X1 nucleotides, wherein at least one nucleotide is modified with a first modification, each of the remaining nucleotides is independently modified with a second modification, and X1 is an integer selected from 13-36, wherein the first modification and the second modification are different; and (b) an antisense strand comprising X2 nucleotides, wherein at least one nucleotide is modified with a third modification, each of the remaining nucleotides is independently modified with a fourth modification, and X2 is an integer selected from 18-31, wherein the third modification and the fourth modification are different.

In some embodiments, the first modification is modification of the sugar moiety of the at least one nucleotide at the 2′-position selected from 2′-F modification, 2′-CN modification, 2′-N3 modification, 2′-deoxy modification, and an equivalent thereof, and a combination thereof. In some embodiments, the first modification is 2′-F modification, 2′-CN modification, 2′-N3 modification, or 2′-deoxy modification, or a stereoisomer thereof. In some embodiments, the first modification is 2′-F modification, 2′-CN modification, or 2′-N3 modification, or a stereoisomer thereof. In some embodiments, the first modification is 2′-F modification or a stereoisomer thereof.

In some embodiments, the second modification is modification of the sugar moiety of one or more of the remaining nucleotides at the 2′-position selected from 2′-C₁-C₆ alkyl, 2′-OR modification wherein R is C1-C6 alkyl optionally substituted with C1-C6 alkoxy, acetamide, phenyl, or heteroaryl comprising a 5- or 6-membered ring and 1 or 2 heteroatoms selected from N, O, and S, 2′-amino, and morpholino replacement, and an equivalent thereof, and a combination thereof. In some embodiments, the second modification is 2′-OR modification, or morpholino replacement, or a combination thereof. In some embodiments, the second modification is 2′-OR modification. In some embodiments, the second modification is 2′-O-methyl modification or 2′-methoxyethoxy modification. In some embodiments, the second modification is 2′-O-methyl modification. In some embodiments, the second modification is morpholino replacement.

In some embodiments, the first modification is 2′-F modification or a stereoisomer thereof, and the second modification is 2′-O-methyl modification or 2′-methoxyethoxy modification. In some embodiments, the first modification is 2′-F modification or a stereoisomer thereof, and the second modification is 2′-O-methyl modification.

In some embodiments, the third modification is modification of the sugar moiety of the at least one nucleotide at the 2′-position selected from 2′-F modification, 2′-CN modification, 2′-N3 modification, 2′-deoxy modification, and an equivalent thereof, and a combination thereof. In some embodiments, the third modification is 2′-F modification, 2′-CN modification, 2′-N3 modification, or 2′-deoxy modification, or a stereoisomer thereof. In some embodiments, the third modification is 2′-F modification, 2′-CN modification, or 2′-N3 modification, or a stereoisomer thereof. In some embodiments, the third modification is 2′-F modification or a stereoisomer thereof.

In some embodiments, the fourth modification is modification of the sugar moiety of one or more of the remaining nucleotides at the 2′-position selected from 2′-C₁-C₆ alkyl, 2′-OR modification wherein R is C₁-C₆ alkyl optionally substituted with C₁-C₆ alkoxy, acetamide, phenyl, or heteroaryl comprising a 5- or 6-membered ring and 1 or 2 heteroatoms selected from N, O, and S, 2′-amino, and morpholino replacement, and an equivalent thereof, and a combination thereof. In some embodiments, the fourth modification is 2′-OR modification, or morpholino replacement, or a combination thereof. In some embodiments, the fourth modification is 2′-OR modification. In some embodiments, the fourth modification is 2′-O-methyl modification or 2′-methoxyethoxy modification. In some embodiments, the fourth modification is 2′-O-methyl modification. In some embodiments, the fourth modification is morpholino replacement.

In some embodiments, the third modification is 2′-F modification or a stereoisomer thereof, and the fourth modification is 2′-O-methyl modification or 2′-methoxyethoxy modification. In some embodiments, the third modification is 2′-F modification or a stereoisomer thereof, and the fourth modification is 2′-O-methyl modification.

In some embodiments, in the sense strand of the isolated oligonucleotide of the present disclosure, at least three nucleotides are modified with the first modification. In some embodiments, in the sense strand of the isolated oligonucleotide of the present disclosure, at least two of the at least three nucleotides modified with the first modification are consecutively located. In some embodiments, in the sense strand of the isolated oligonucleotide of the present disclosure, at least three of the at least three nucleotides modified with the first modification are consecutively located.

In some embodiments, in the sense strand of the isolated oligonucleotide of the present disclosure, in the sense strand at least four nucleotides are modified with the first modification. In some embodiments, in the sense strand of the isolated oligonucleotide of the present disclosure, at least three of the at least four nucleotides modified with the first modification are consecutively located. In some embodiments, in the sense strand of the isolated oligonucleotide of the present disclosure, at least four of the at least four nucleotides modified with the first modification are consecutively located.

In some embodiments, in the sense strand of the isolated oligonucleotide of the present disclosure, in the sense strand at least five nucleotides are modified with the first modification. In some embodiments, in the sense strand of the isolated oligonucleotide of the present disclosure, at least three of the at least five nucleotides modified with the first modification are consecutively located. at least four of the at least five nucleotides modified with the first modification are consecutively located.

In some embodiments, in the sense strand of the isolated oligonucleotide of the present disclosure, the at least three nucleotides, the at least four nucleotides, or the at least five nucleotides modified with the first modification are located from position 10 to position 15 from the nucleotide complementary to the first nucleotide at the 5′-terminus of the antisense strand.

In some embodiments, in the sense strand of the isolated oligonucleotide of the present disclosure, two of the at least three nucleotides modified with the first modification are located at positions selected from position 10, 11, 12, and 13 from the nucleotide complementary to the first nucleotide at the 5′-terminus of the antisense strand. In some embodiments, in the sense strand of the isolated oligonucleotide of the present disclosure, three of the at least three nucleotides modified with the first modification are located at positions selected from position 10, 11, 12, and 13 from the nucleotide complementary to the first nucleotide at the 5′-terminus of the antisense strand.

In some embodiments, in the sense strand of the isolated oligonucleotide of the present disclosure, one of the at least three nucleotides modified with the first modification is located at position 11 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 10 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 14 from the nucleotide complementary to the first nucleotide at the 5′-terminus of the antisense strand.

In some embodiments, in the sense strand of the isolated oligonucleotide of the present disclosure, two of the at least four nucleotides modified with the first modification are located from position 10 to position 14 from the nucleotide complementary to the first nucleotide at the 5′-terminus of the antisense strand.

In some embodiments, in the sense strand of the isolated oligonucleotide of the present disclosure, not all of the at least three nucleotides modified with the first modification are consecutively located. In some embodiments, in the sense strand of the isolated oligonucleotide of the present disclosure, the at least three nucleotides, the at least four nucleotides, or the at least five nucleotides are modified with 2′-F modification.

In some embodiments, the sense strand of the isolated oligonucleotide of the present disclosure, comprises nucleotides modified with 2′-F modification, and nucleotides modified with 2′-O-methyl modification, according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and each of a, b, c, d, e, f and g is any one of 0-16, and indicates the number of consecutive nucleotides modified with the modification, and wherein the sense strand is any one of:

| | a) |
|---|---|
| (SEQ ID NO: 52) | |
| | 5′(M)₀(F)₀(M)₀(F)₀(M)₈(F)₃(M)₁₀ 3′; |
| | b) |
| (SEQ ID NO: 53) | |
| | 5′(M)₀(F)₀(M)₅(F)₁(M)₁(F)₃(M)₁₀ 3′; |
| | c) |
| (SEQ ID NO: 54) | |
| | 5′(M)₀(F)₀(M)₀(F)₀(M)₇(F)₃(M)₁₀ 3′; |
| | d) |
| (SEQ ID NO: 55) | |
| | 5′(M)₀(F)₀(M)₅(F)₁(M)₁(F)₂(M)₁₁ 3′; |
| | e) |
| (SEQ ID NO: 56) | |
| | 5′(M)₀(F)₀(M)₆(F)₁(M)₁(F)₂(M)₁₁ 3′; |
| | f) |
| (SEQ ID NO: 57) | |
| | 5′(M)₆(F)₁(M)₁(F)₂(M)₁(F)₁(M)₉ 3′; |
| | g) |
| (SEQ ID NO: 58) | |
| | 5′(M)₅(F)₁(M)₁(F)₂(M)₁(F)₁(M)₉ 3′; |
| | h) |
| (SEQ ID NO: 59) | |
| | 5′(M)₀(F)₀(M)₀(F)₀(M)₇(F)₄(M)₁₀ 3′; |
| | i) |
| (SEQ ID NO: 60) | |
| | 5′(M)₀(F)₀(M)₆(F)₁(M)₁(F)₄(M)₉ 3′; |
| | j) |
| (SEQ ID NO: 61) | |
| | 5′(M)₀(F)₀(M)₀(F)₀(M)₈(F)₄(M)₉ 3′; |
| | k) |
| (SEQ ID NO: 62) | |
| | 5′(M)₀(F)₀(M)₀(F)₀(M)₆(F)₄(M)₁₀ 3′; |
| | l) |
| (SEQ ID NO: 63) | |
| | 5′(M)₀(F)₀(M)₅(F)₁(M)₁(F)₄(M)₉ 3′; |
| | m) |
| (SEQ ID NO: 64) | |
| | 5′(M)₀(F)₀(M)₀(F)₀(M)(F)₄(M)₉ 3′; |
| | n) |
| (SEQ ID NO: 65) | |
| | 5′(M)₀(F)₀(M)₀(F)₀(M)₇(F)₅(M)₉ 3′; |
| | and |
| | o) |
| (SEQ ID NO: 66) | |
| | 5′(M)₀(F)₀(M)₀(F)₀(M)₆(F)₅(M)₉ 3′. |

In some embodiments, in the antisense strand of the isolated oligonucleotide of the present disclosure, at most seven nucleotides are modified with the third modification.

In some embodiments, in the antisense strand of the isolated oligonucleotide of the present disclosure, at most four of the at most seven nucleotides modified with the third modification are located from position 2 to position 8 from the first nucleotide at the 5′-terminus of the antisense strand. In some embodiments, in the antisense strand of the isolated oligonucleotide of the present disclosure, at most two of the at most seven nucleotides modified with the third modification are consecutively located.

In some embodiments, in the antisense strand of the isolated oligonucleotide of the present disclosure, three or four of the at most seven nucleotides modified with the third modification are located at positions selected from position 2, 3, 5, 6, 7, and 8 from the first nucleotide at the 5′-terminus of the antisense strand. In some embodiments, in the antisense strand of the isolated oligonucleotide of the present disclosure, at least one of the at most seven nucleotides modified with the third modification is located at position 14 from the first nucleotide at the 5′-terminus of the antisense strand. In some embodiments, in the antisense strand of the isolated oligonucleotide of the present disclosure, two or three of the at most seven nucleotides modified with the third modification are located at positions selected from position 2, 3, 5, and 6 from the first nucleotide at the 5′-terminus of the antisense strand. In some embodiments, in the antisense strand of the isolated oligonucleotide of the present disclosure, three of the at most seven nucleotides modified with the third modification are located at positions selected from position 2, 3, 5, and 6 from the first nucleotide at the 5′-terminus of the antisense strand.

In some embodiments, in the antisense strand of the isolated oligonucleotide of the present disclosure, two of the at most seven nucleotides modified with the third modification are located at positions 8 and 14 from the nucleotide complementary to the first nucleotide at the 5′-terminus of the antisense strand.

In some embodiments, in the antisense strand of the isolated oligonucleotide of the present disclosure, one or two of the at most seven nucleotides modified with the third modification are located at positions selected from position 14 and 16 from the first nucleotide at the 5′-terminus of the antisense strand. In some embodiments, in the antisense strand of the isolated oligonucleotide of the present disclosure, two of the at most seven nucleotides modified with the third modification are located at positions 14 and 16 from the first nucleotide at the 5′-terminus of the antisense strand. In some embodiments, in the antisense strand of the isolated oligonucleotide of the present disclosure, the at most seven nucleotides are modified with 2′-F modification.

In some embodiments, in the antisense strand of the isolated oligonucleotide of the present disclosure, at most six nucleotides are modified with the third modification. In some embodiments, in the antisense strand of the isolated oligonucleotide of the present disclosure, the at most six nucleotides are modified with 2′-F modification.

In some embodiments, in the antisense strand of the isolated oligonucleotide of the present disclosure, at most five nucleotides are modified with the third modification. In some embodiments, in the antisense strand of the isolated oligonucleotide of the present disclosure, the at most five nucleotides are modified with 2′-F modification.

In some embodiments, in the antisense strand of the isolated oligonucleotide of the present disclosure, at most four nucleotides are modified with the third modification. In some embodiments, in the antisense strand of the isolated oligonucleotide of the present disclosure, at most four nucleotides are modified with 2′-F modification. In some embodiments, in the antisense strand of the isolated oligonucleotide of the present disclosure, at most three nucleotides are modified with the third modification. In some embodiments, in the antisense strand of the isolated oligonucleotide of the present disclosure, the at least three nucleotides are modified with 2′-F modification.

In some embodiments, in the antisense strand of the isolated oligonucleotide of the present disclosure, the antisense strand comprises nucleotides modified with 2′-F modification, and nucleotides modified with 2′-O-methyl modification, according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and each of a, b, c, d, e, f, g, h, i, j, k, l, m, n and o is any one of 0-16, and indicated the number of consecutive nucleotides modified with the modification, and wherein the antisense strand is any one of:

1)
(SEQ ID NO: 14)
3′(M)₀(F)₀(M)₀(F)₀(M)₀(F)₀(M)₇(F)₁(M)₁(F)₁(M)₇(F)₂(M)₂(F)₁(M)₁ 5′;
2)
(SEQ ID NO: 15)
3′(M)₀(F)₀(M)₀(F)₀(M)₀(F)₀(M)₇(F)₁(M)₁(F)₁(M)₇(F)₁(M)₂(F)₂(M)₁ 5′;
3)
(SEQ ID NO: 16)
3′(M)₀(F)₀(M)₀(F)₀(M)₀(F)₀(M)₇(F)₁(M)₁(F)₁(M)₆(F)₂(M)₃(F)₁(M)₁ 5′;
4)
(SEQ ID NO: 17)
3′(M)₀(F)₀(M)₀(F)₀(M)₇(F)₁(M)₁(F)₁(M)₅(F)₁(M)₁(F)₁(M)₃(F)₁(M)₁ 5′;
5)
(SEQ ID NO: 18)
3′(M)₀(F)₀(M)₀(F)₀(M)₇(F)₁(M)₁(F)₁(M)₄(F)₁(M)₂(F)₁(M)₃(F)₁(M)₁ 5′;
6)
(SEQ ID NO: 20)
3′(M)₀(F)₀(M)₀(F)₀(M)₀(F)₀(M)₇(F)₁(M)₁(F)₁(M)₈(F)₁(M)₂(F)₁(M)₁ 5′;
7)
(SEQ ID NO: 21)
3′(M)₀(F)₀(M)₀(F)₀(M)₀(F)₀(M)₀(F)₀(M)₉(F)₁(M)₇(F)₁(M)₃(F)₁(M)₁ 5′;
8)
(SEQ ID NO: 22)
3′(M)₀(F)₀(M)₀(F)₀(M)₇(F)₁(M)₁(F)₁(M)₄(F)₂(M)₁(F)₁(M)₂(F)₂(M)₁ 5′;
9)
(SEQ ID NO: 23)
3′(M)₀(F)₀(M)₀(F)₀(M)₇(F)₁(M)₁(F)₁(M)₄(F)₂(M)₂(F)₁(M)₁(F)₂(M)₁ 5′;
10)
(SEQ ID NO: 24)
3′(M)₀(F)₀(M)₀(F)₀(M)₇(F)₁(M)₁(F)₁(M)₄(F)₁(M)₁(F)₁(M)₁(F)₂(M)₁ 5′;
11)
(SEQ ID NO: 25)
3′(M)₀(F)₀(M)₀(F)₀(M)₇(F)₁(M)₁(F)₁(M)₃(F)₁(M)₂(F)₁(M)₁(F)₁(M)₁(F)₂(M)₁ 5′;
12)
(SEQ ID NO: 27)
3′(M)₀(F)₀(M)₄(F)₁(M)₁(F)₁(M)₁(F)₁(M)₃(F)₁(M)₁(F)₁(M)₁(F)₃(M)₁(F)₁(M)₁ 5′;
13)
(SEQ ID NO: 30)
3′(M)₀(F)₀(M)₀(F)₀(M)₀(F)₀(M)₆(F)₁(M)₁(F)₁(M)₇(F)₁(M)₃(F)₁(M)₁ 5′;
14)
(SEQ ID NO: 50)
3′(M)₀(F)₀(M)₀(F)₀(M)₀(F)₀(M)₆(F)₁(M)₁(F)₁(M)₇(F)₂(M)₂(F)₁(M)₁ 5′;
15)
(SEQ ID NO: 45)
3′(M)₀(F)₀(M)₀(F)₀(M)₀(F)₀(M)₆(F)₁(M)₁(F)₁(M)₇(F)₁(M)₂(F)₂(M)₁ 5′;
16)
(SEQ ID NO: 46)
3′(M)₀(F)₀(M)₀(F)₀(M)₀(F)₀(M)₆(F)₁(M)₁(F)₁(M)₆(F)₂(M)₃(F)₁(M)₁ 5′;
17)
(SEQ ID NO: 47)
3′(M)₀(F)₀(M)₀(F)₀(M)₆(F)₁(M)₁(F)₁(M)₅(F)₁(M)₁(F)₁(M)₃(F)₁(M)₁ 5′;
18)
(SEQ ID NO: 48)
3′(M)₀(F)₀(M)₀(F)₀(M)₆(F)₁(M)₁(F)₁(M)₄(F)₁(M)₂(F)₁(M)₃(F)₁(M)₁ 5′;
19)
(SEQ ID NO: 49)
3′(M)₀(F)₀(M)₀(F)₀(M)₆(F)₁(M)₁(F)₁(M)₄(F)₂(M)₁(F)₁(M)₃(F)₁(M)₁ 5′;
20)
(SEQ ID NO: 37)
3′(M)₀(F)₀(M)₀(F)₀(M)₀(F)₀(M)₆(F)₁(M)₁(F)₁(M)₈(F)₁(M)₂(F)₁(M)₁ 5′;
21)
(SEQ ID NO: 38)
3′(M)₀(F)₀(M)₀(F)₀(M)₀(F)₀(M)₀(F)₀(M)₈(F)₁(M)₇(F)₁(M)₃(F)₁(M)₁ 5′;
22)
(SEQ ID NO: 39)
3′(M)₀(F)₀(M)₀(F)₀(M)₆(F)₁(M)₁(F)₁(M)₄(F)₂(M)₁(F)₁(M)₂(F)₂(M)₁ 5′;
23)
(SEQ ID NO: 40)
3′(M)₀(F)₀(M)₀(F)₀(M)₆(F)₁(M)₁(F)₁(M)₄(F)₂(M)₂(F)₁(M)₁(F)₂(M)₁ 5′;
24)
(SEQ ID NO: 41)
3′(M)₀(F)₀(M)₆(F)₁(M)₁(F)₁(M)₄(F)₁(M)₁(F)₁(M)₁(F)₁(M)₁(F)₂(M)₁ 5′;
and
25)
(SEQ ID NO: 42)
3′(M)₀(F)₀(M)₆(F)₁(M)₁(F)₁(M)₃(F)₁(M)₂(F)₁(M)₁(F)₁(M)₁(F)₂(M)₁ 5′.

In some embodiments, the X1 nucleotides of the sense strand of the isolated oligonucleotide of the present disclosure, is 18-21 and the X2 nucleotides of the antisense strand of the isolated oligonucleotide of the present disclosure is 20-23. In some embodiments, the X1 nucleotides of the sense strand of the isolated oligonucleotide of the present disclosure, is 20 or 21 and the X2 nucleotides of the antisense strand of the isolated oligonucleotide of the present disclosure is 22 or 23. In some embodiments, the X2 nucleotides of the antisense strand of the isolated oligonucleotide of the present disclosure equals the X1 nucleotides of the sense strand of the isolated oligonucleotide of the present disclosure plus 2.

In some embodiments, the sense strand of the isolated oligonucleotide of the present disclosure comprises at least one nucleotide having a modified phosphate backbone. In some embodiments, the antisense strand of the isolated oligonucleotide of the present disclosure comprises at least one nucleotide having a modified phosphate backbone. In some embodiments, in the sense strand or the antisense strand or both sense and antisense strands of the isolated oligonucleotide of the present disclosure, the modified phosphate backbone comprises a modified phosphodiester bond. In some embodiments, the modified phosphodiester bond is modified by replacing one or more oxygen atoms with a moiety, wherein the moiety is bonded to the phosphorus atom in the phosphodiester bond with a carbon, nitrogen, or sulfur atom in the moiety, or by forming a 2′-5′ linkage. In some embodiments, the modified phosphodiester bond comprises phosphorothioate, phosphorodithioate, methylphosphonate, phosphoramidate diester, mesyl phosphoramidate, or phosphonoacetate.

In some embodiments, the isolated oligonucleotide of the present disclosure comprises one or more non-natural base-containing nucleotide, a locked nucleotide, or an abasic nucleotide. In some embodiments, the isolated oligonucleotide of the present disclosure, the terminal nucleotide at the 5′ end comprises a phosphate mimic. In some embodiments, the 5′-phosphate mimic is ethylphosphonate, vinylphosphonate or an analog thereof

In some embodiments, the antisense strand of the isolated oligonucleotide of the present disclosure comprises an overhang with at least two single-stranded nucleotides at the 3′-terminus.

In some embodiments, in the sense strand or the antisense strand or both of the isolated oligonucleotide of the present disclosure, a terminal or internal nucleotide is linked to one or more targeting ligands. In some embodiments, the terminal or internal nucleotide is linked to the one or more targeting ligands directly. In some embodiments, the terminal or internal nucleotide is linked to the one or more targeting ligands indirectly by a linker. In some embodiments, the one or more targeting ligands linked directly or indirectly to the terminal or internal nucleotide can further comprise a PK modulator. In some embodiments, the PK modulator is a competitive modulator, a positive allosteric modulator, a negative allosteric modulator or a neutral allosteric modulator. In some embodiments, the targeting ligand is selected from one or more of a carbohydrate, a peptide, a lipid, an antibody or a fragment thereof, an aptamer, an albumin, a fibrinogen, and a folate.

In some embodiments, the antisense strand of the isolated oligonucleotide of the present disclosure is complementary to an mRNA, wherein the sequence-specific hybridization to the antisense strand to the mRNA results in degradation of the mRNA.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF DRAWINGS

FIG. 1 is a chart depicting a set of siRNA 21/23 mer molecules directed to target gene 1 with different chemical modifications as indicated.

FIGS. 2A-2C are graphs depicting the potency of the siRNA molecules of FIG. 1. The X-axis indicates increasing concentration of the siRNAs and the Y-axis indicates the remaining % of the human mRNA of target gene 1. The IC₅₀ values are as indicated.

FIG. 3 is a chart depicting a set of siRNA 21/23 mer molecules which are optimized on the sense strand (e.g., at positions 7, 8, and 12) with the chemical modifications as indicated.

FIGS. 4A-4E are graphs depicting the potency of the siRNA molecules of FIG. 3. The X-axis indicates increasing concentration of the siRNAs and the Y-axis indicates the remaining % of the human mRNA of target gene 1. The IC₅₀ values are as indicated.

FIG. 5 is a chart depicting a set of siRNA 21/23 mer molecules which are optimized on the sense strands (e.g., at positions 11 and 12) with the chemical modifications as indicated.

FIGS. 6A-6C are graphs depicting the potency of the siRNA molecules of FIG. 5. The X-axis indicates increasing concentration of the siRNAs and the Y-axis indicates the remaining % of the human mRNA of target gene 1. The IC₅₀ values are as indicated.

FIG. 7 is a chart depicting a set of siRNA 21/23 mer molecules which are optimized on the antisense strand (e.g., at positions 3, 5, and 7-9) with the chemical modifications as indicated.

FIGS. 8A-8F are graphs depicting the potency of the siRNA molecules of FIG. 7. The X-axis indicates increasing concentration of the siRNAs and the Y-axis indicates the remaining % of the human mRNA of target gene 1. The IC₅₀ values are as indicated.

FIG. 9 is a chart depicting a set of siRNA 21/23 mer molecules which are optimized on the antisense strand (e.g., at the seed region and position 16) with the chemical modifications as indicated.

FIGS. 10A-10C are graphs depicting the potency of the siRNA molecules of FIG. 9. The X-axis indicates increasing concentration of the siRNAs and the Y-axis indicates the remaining % of the human mRNA of target gene 1. The IC₅₀ values are as indicated.

FIG. 11 is a chart depicting a set of siRNA 21/23 mer molecules which are optimized on both the sense and antisense strands with the chemical modifications as indicated.

FIGS. 12A-12F are graphs depicting the potency of the siRNA molecules of FIG. 11. The X-axis indicates increasing concentration of the siRNAs and the Y-axis indicates the remaining % of the human mRNA of target gene 1. The IC₅₀ values are as indicated.

FIG. 13 is a chart depicting a set of siRNA 20/22 mer molecules which are optimized with the chemical modifications as indicated.

FIGS. 14A-14C are graphs depicting the potency of the siRNA molecules of FIG. 13. The X-axis indicates increasing concentration of the siRNAs and the Y-axis indicates the remaining % of the human mRNA of target gene 1. The IC₅₀ values are as indicated.

FIG. 15 is a chart depicting a set of siRNA 20/22 mer molecules which are optimized on the sense strand (e.g., at positions 6, 7, and 11) with the chemical modifications as indicated.

FIGS. 16A-16E are graphs depicting the potency of the siRNA molecules of FIG. 15. The X-axis indicates increasing concentration of the siRNAs and the Y-axis indicates the remaining % of the human mRNA of target gene 1. The IC₅₀ values are as indicated.

FIG. 17 is a chart depicting a set of siRNA 20/22 mer molecules which are optimized on the sense strand (e.g., at positions 10 and 11) with the chemical modifications as indicated.

FIGS. 18A-18C are graphs depicting the potency of the siRNA molecules of FIG. 17. The X-axis indicates increasing concentration of the siRNAs and the Y-axis indicates the remaining % of the human mRNA of target gene 1. The IC₅₀ values are as indicated.

FIG. 19 is a chart depicting a set of siRNA 20/22 mer molecules which are optimized on the antisense strand (e.g., at positions 3, 5, and 7-9) with the chemical modifications as indicated.

FIGS. 20A-20F are graphs depicting the potency of the siRNA molecules of FIG. 19. The X-axis indicates increasing concentration of the siRNAs and the Y-axis indicates the remaining % of the human mRNA of target gene 1. The IC₅₀ values are as indicated.

FIG. 21 is a chart depicting a set of siRNA 20/22 mer molecules which are optimized on the antisense strand with the chemical modifications as indicated.

FIGS. 22A-22C are graphs depicting the potency of the siRNA molecules of FIG. 21. The X-axis indicates increasing concentration of the siRNAs and the Y-axis indicates the remaining % of the human mRNA of target gene 1. The IC₅₀ values are as indicated.

FIG. 23 is a chart depicting a set of siRNA 20/22 mer molecules which are optimized on both the sense and antisense strands with the chemical modifications as indicated.

FIGS. 24A-24F are graphs depicting the potency of the siRNA molecules of FIG. 23. The X-axis indicates increasing concentration of the siRNAs and the Y-axis indicates the remaining % of the human mRNA of target gene 1. The IC₅₀ values are as indicated.

### DETAILED DESCRIPTION

Provided herein is an isolated oligonucleotide, comprising: (a) a sense strand comprising X1 nucleotides, wherein at least one nucleotide is modified with a first modification, each of the remaining nucleotides is independently modified with a second modification, and X1 is an integer selected from 13-36, wherein the first modification and the second modification are different; and (b) an antisense strand comprising X2 nucleotides, wherein at least one nucleotide is modified with a third modification, each of the remaining nucleotides is independently modified with a fourth modification, and X2 is an integer selected from 18-31, wherein the third modification and the fourth modification are different.

In some embodiments, the first modification is modification of the sugar moiety of the at least one nucleotide at the 2′-position selected from 2′-F modification, 2′-CN modification, 2′-N3 modification, 2′-deoxy modification, and an equivalent thereof, and a combination thereof. In some embodiments, the first modification is 2′-F modification, 2′-CN modification, 2′-N3 modification, or 2′-deoxy modification, or a stereoisomer thereof. In some embodiments, the first modification is 2′-F modification, 2′-CN modification, or 2′-N3 modification, or a stereoisomer thereof. In some embodiments, the first modification is 2′-F modification or a stereoisomer thereof.

In some embodiments, the second modification is modification of the sugar moiety of one or more of the remaining nucleotides at the 2′-position selected from 2′-C1-C6 alkyl, 2′-OR modification wherein R is C1-C6 alkyl optionally substituted with C1-C6 alkoxy, acetamide, phenyl, or heteroaryl comprising a 5- or 6-membered ring and 1 or 2 heteroatoms selected from N, O, and S, 2′-amino, and morpholino replacement, and an equivalent thereof, and a combination thereof. In some embodiments, the second modification is 2′-OR modification, or morpholino replacement, or a combination thereof. In some embodiments, the second modification is 2′-OR modification. In some embodiments, the second modification is 2′-O-methyl modification or 2′-methoxyethoxy modification. In some embodiments, the second modification is 2′-O-methyl modification. In some embodiments, the second modification is morpholino replacement.

In some embodiments, the first modification is 2′-F modification or a stereoisomer thereof, and the second modification is 2′-O-methyl modification or 2′-methoxyethoxy modification. In some embodiments, the first modification is 2′-F modification or a stereoisomer thereof, and the second modification is 2′-O-methyl modification.

In some embodiments, the third modification is modification of the sugar moiety of the at least one nucleotide at the 2′-position selected from 2′-F modification, 2′-CN modification, 2′-N3 modification, 2′-deoxy modification, and an equivalent thereof, and a combination thereof. In some embodiments, the third modification is 2′-F modification, 2′-CN modification, 2′-N3 modification, or 2′-deoxy modification, or a stereoisomer thereof. In some embodiments, the third modification is 2′-F modification, 2′-CN modification, or 2′-N3 modification, or a stereoisomer thereof. In some embodiments, the third modification is 2′-F modification or a stereoisomer thereof.

In some embodiments, the fourth modification is modification of the sugar moiety of one or more of the remaining nucleotides at the 2′-position selected from 2′-C₁-C₆ alkyl, 2′-OR modification wherein R is C₁-C₆ alkyl optionally substituted with C₁-C₆ alkoxy, acetamide, phenyl, or heteroaryl comprising a 5- or 6-membered ring and 1 or 2 heteroatoms selected from N, O, and S, 2′-amino, and morpholino replacement, and an equivalent thereof, and a combination thereof. In some embodiments, the fourth modification is 2′-OR modification, or morpholino replacement, or a combination thereof. In some embodiments, the fourth modification is 2′-OR modification. In some embodiments, the fourth modification is 2′-O-methyl modification or 2′-methoxyethoxy modification. In some embodiments, the fourth modification is 2′-O-methyl modification. In some embodiments, the fourth modification is morpholino replacement.

In some embodiments, the third modification is 2′-F modification or a stereoisomer thereof, and the fourth modification is 2′-O-methyl modification or 2′-methoxyethoxy modification. In some embodiments, the third modification is 2′-F modification or a stereoisomer thereof, and the fourth modification is 2′-O-methyl modification.

In some embodiments, in the sense strand of the isolated oligonucleotide of the present disclosure, at least three nucleotides are modified with the first modification. In some embodiments, in the sense strand of the isolated oligonucleotide of the present disclosure, at least two of the at least three nucleotides modified with the first modification are consecutively located. In some embodiments, in the sense strand of the isolated oligonucleotide of the present disclosure, at least three of the at least three nucleotides modified with the first modification are consecutively located.

In some embodiments, in the sense strand of the isolated oligonucleotide of the present disclosure, in the sense strand at least four nucleotides are modified with the first modification. In some embodiments, in the sense strand of the isolated oligonucleotide of the present disclosure, at least three of the at least four nucleotides modified with the first modification are consecutively located. In some embodiments, in the sense strand of the isolated oligonucleotide of the present disclosure, at least four of the at least four nucleotides modified with the first modification are consecutively located.

In some embodiments, in the sense strand of the isolated oligonucleotide of the present disclosure, in the sense strand at least five nucleotides are modified with the first modification. In some embodiments, in the sense strand of the isolated oligonucleotide of the present disclosure, at least three of the at least five nucleotides modified with the first modification are consecutively located. at least four of the at least five nucleotides modified with the first modification are consecutively located.

In some embodiments, in the sense strand of the isolated oligonucleotide of the present disclosure, the at least three nucleotides, the at least four nucleotides, or the at least five nucleotides modified with the first modification are located from position 10 to position 15 from the nucleotide complementary to the first nucleotide at the 5′-terminus of the antisense strand.

In some embodiments, in the sense strand of the isolated oligonucleotide of the present disclosure, two of the at least three nucleotides modified with the first modification are located at positions selected from position 10, 11, 12, and 13 from the nucleotide complementary to the first nucleotide at the 5′-terminus of the antisense strand. In some embodiments, in the sense strand of the isolated oligonucleotide of the present disclosure, three of the at least three nucleotides modified with the first modification are located at positions selected from position 10, 11, 12, and 13 from the nucleotide complementary to the first nucleotide at the 5′-terminus of the antisense strand. In some embodiments, in the sense strand of the isolated oligonucleotide of the present disclosure, one of the at least three nucleotides modified with the first modification is located at position 11 from the nucleotide complementary to the first nucleotide at the 5′-terminus of the antisense strand.

In some embodiments, in the sense strand of the isolated oligonucleotide of the present disclosure, three of the at least three nucleotides modified with the first modification are located at positions 11, 12 and 13 from the nucleotide complementary to the first nucleotide at the 5′-terminus of the antisense strand. In some embodiments, in the sense strand of the isolated oligonucleotide of the present disclosure, three of the at least three nucleotides modified with the first modification are located at positions 12, 13 and 14 from the nucleotide complementary to the first nucleotide at the 5′-terminus of the antisense. In some embodiments, in the sense strand of the isolated oligonucleotide of the present disclosure, three of the at least three nucleotides modified with the first modification are located at positions 10, 11 and 12 from the nucleotide complementary to the first nucleotide at the 5′-terminus of the antisense strand.

In some embodiments, in the sense strand of the isolated oligonucleotide of the present disclosure, one of the at least four nucleotides modified with the first modification is located at position 10 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 11 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 12 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 13 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 14 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 15 from the nucleotide complementary to the first nucleotide at the 5′-terminus of the antisense strand.

In some embodiments, in the sense strand of the isolated oligonucleotide of the present disclosure, the at least four nucleotides modified with the first modification are located at positions 11, 12, 13 and 14 from the nucleotide complementary to the first nucleotide at the 5′-terminus of the antisense strand. In some embodiments, in the sense strand of the isolated oligonucleotide of the present disclosure, the at least four nucleotides modified with the first modification are located at positions 10, 11, 12 and 13 from the nucleotide complementary to the first nucleotide at the 5′-terminus of the antisense strand. In some embodiments, in the sense strand of the isolated oligonucleotide of the present disclosure, the at least four nucleotides modified with the first modification are located at positions 11, 12, 13 and 15 from the nucleotide complementary to the first nucleotide at the 5′-terminus of the antisense strand.

In some embodiments, in the sense strand of the isolated oligonucleotide of the present disclosure, two of the at least four nucleotides modified with the first modification are located from position 10 to position 14 from the nucleotide complementary to the first nucleotide at the 5′-terminus of the antisense strand.

In some embodiments, in the sense strand of the isolated oligonucleotide of the present disclosure, not all of the at least three nucleotides modified with the first modification are consecutively located. In some embodiments, in the sense strand of the isolated oligonucleotide of the present disclosure, the at least three nucleotides, the at least four nucleotides, or the at least five nucleotides are modified with 2′-F modification.

In some embodiments, in the sense strand of the isolated oligonucleotide of the present disclosure, the at least three nucleotides modified with the first modification are located at positions 12, 13 and 15 from the nucleotide complementary to the first nucleotide at the 5′-terminus of the antisense strand (e.g., SEQ ID NO: 11, SEQ ID NO: 34). In some embodiments, in the sense strand of the isolated oligonucleotide of the present disclosure, the at least four nucleotides modified with the first modification are located at positions 10, 12, 13 and 15 from the nucleotide complementary to the first nucleotide at the 5′-terminus of the antisense strand (e.g., SEQ ID NO: 12, SEQ ID NO: 35). In some embodiments, in the sense strand of the isolated oligonucleotide of the present disclosure, the at least four nucleotides modified with the first modification are located at positions 11, 12, 13 and 14 from the nucleotide complementary to the first nucleotide at the 5′-terminus of the antisense strand (e.g., SEQ ID NO: 8, SEQ ID NO: 32). In some embodiments, in the sense strand of the isolated oligonucleotide of the present disclosure, the at least four nucleotides modified with the first modification are located at positions 10, 11, 12 and 13 from the nucleotide complementary to the first nucleotide at the 5′-terminus of the antisense strand (e.g., SEQ ID NO: 9, SEQ ID NO: 33). In some embodiments, in the sense strand of the isolated oligonucleotide of the present disclosure, the at least five nucleotides modified with the first modification are located at positions 10, 11, 12, 13 and 14 from the nucleotide complementary to the first nucleotide at the 5′-terminus of the antisense strand (e.g., SEQ ID NO: 10, SEQ ID NO: 36). In some embodiments, in the sense strand of the isolated oligonucleotide of the present disclosure, the at least five nucleotides modified with the first modification are located at positions 10, 11, 12, 13, and 15 from the nucleotide complementary to the first nucleotide at the 5′-terminus of the antisense strand (e.g., SEQ ID NO: 13, SEQ ID NO: 44).

In some embodiments, the sense strand of the isolated oligonucleotide of the present disclosure, comprises nucleotides modified with 2′-F modification, and nucleotides modified with 2′-O-methyl modification, according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and each of a, b, c, d, e, f and g is any one of 0-16, and indicates the number of consecutive nucleotides modified with the modification, and wherein the sense strand is any one of:

| | a) |
|---|---|
| (SEQ ID NO: 52) | |
| | 5′(M)₀(F)₀(M)₀(F)₀(M)₈(F)₃(M)₁₀ 3′; |
| | b) |
| (SEQ ID NO: 53) | |
| | 5′(M)₀(F)₀(M)₅(F)₁(M)₁(F)₃(M)₁₀ 3′; |
| | c) |
| (SEQ ID NO: 54) | |
| | 5′(M)₀(F)₀(M)₀(F)₀(M)₇(F)₃(M)₁₀ 3′; |
| | d) |
| (SEQ ID NO: 55) | |
| | 5′(M)₀(F)₀(M)₅(F)₁(M)₁(F)₂(M)₁₁ 3′; |
| | e) |
| (SEQ ID NO: 56) | |
| | 5′(M)₀(F)₀(M)₆(F)₁(M)₁(F)₂(M)₁₁ 3′; |
| | f) |
| (SEQ ID NO: 57) | |
| | 5′(M)₆(F)₁(M)₁(F)₂(M)₁(F)₁(M)₉ 3′; |
| | g) |
| (SEQ ID NO: 58) | |
| | 5′(M)₅(F)₁(M)₁(F)₂(M)₁(F)₁(M)₉ 3′; |
| | h) |
| (SEQ ID NO: 59) | |
| | 5′(M)₀(F)₀(M)₀(F)₀(M)₇(F)₄(M)₁₀ 3′; |
| | i) |
| (SEQ ID NO: 60) | |
| | 5′(M)₀(F)₀(M)₆(F)₁(M)₁(F)₄(M)₉ 3′; |
| | j) |
| (SEQ ID NO: 61) | |
| | 5′(M)₀(F)₀(M)₀(F)₀(M)₈(F)₄(M)₉ 3′; |
| | k) |
| (SEQ ID NO: 62) | |
| | 5′(M)₀(F)₀(M)₀(F)₀(M)₆(F)₄(M)₁₀ 3′; |
| | l) |
| (SEQ ID NO: 63) | |
| | 5′(M)₀(F)₀(M)₅(F)₁(M)₁(F)₄(M)₉ 3′; |
| | m) |
| (SEQ ID NO: 64) | |
| | 5′(M)₀(F)₀(M)₀(F)₀(M)₇(F)₄(M)₉ 3′; |
| | n) |
| (SEQ ID NO: 65) | |
| | 5′(M)₀(F)₀(M)₀(F)₀(M)₇(F)₅(M)₉ 3′; |
| | and |
| | o) |
| (SEQ ID NO: 66) | |
| | 5′(M)₀(F)₀(M)₀(F)₀(M)₆(F)₅(M)₉ 3′. |

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₀(F)₀(M)₈(F)₃(M)₁₀ 3′ (SEQ ID NO: 52), wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₃(M)₁₀ 3′ (SEQ ID NO: 53), wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₀(F)₀(M)₇(F)₃(M)₁₀ 3′ (SEQ ID NO: 54), wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)ᵢ(M)ᵢ(F)₂(M)₁₁ 3′ (SEQ ID NO: 55), wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₆(F)₁(M)₁(F)₂(M)₁₁ 3′ (SEQ ID NO: 56), wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₆(F)₁(M)₁(F)₂(M)₁(F)₁(M)₉ 3′ (SEQ ID NO: 57), wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₅(F)₁(M)₁(F)₂(M)₁(F)₁(M)₉ 3′ (SEQ ID NO: 58), wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₀(F)₀(M)₇(F)₄(M)₁₀ 3′ (SEQ ID NO: 59), wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₆(F)₁(M)₁(F)₄(M)₉ 3′ (SEQ ID NO: 60), wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₀(F)₀(M)₈(F)₄(M)₉ 3′ (SEQ ID NO: 61), wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₀(F)₀(M)₆(F)₄(M)₁₀ 3′ (SEQ ID NO: 62), wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₄(M)₉ 3′ (SEQ ID NO: 63), wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₀(F)₀(M)₇(F)₄(M)₉ 3′ (SEQ ID NO: 64), wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₀(F)₀(M)₇(F)₅(M)₉ 3′ (SEQ ID NO: 65), wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₀(F)₀(M)₆(F)₅(M)₉ 3′ (SEQ ID NO: 66), wherein M is 2′-O-methyl modified nucleotide, F is 2′-F modified nucleotide.

In some embodiments, in the antisense strand of the isolated oligonucleotide of the present disclosure, at most seven nucleotides are modified with the third modification.

In some embodiments, in the antisense strand of the isolated oligonucleotide of the present disclosure, at most four of the at most seven nucleotides modified with the third modification are located from position 2 to position 8 from the first nucleotide at the 5′-terminus of the antisense strand. In some embodiments, in the antisense strand of the isolated oligonucleotide of the present disclosure, at least one of the at most seven nucleotides are modified with the third modification is located at position 2 from the first nucleotide at the 5′-terminus of the antisense strand.

In some embodiments, in the antisense strand of the isolated oligonucleotide of the present disclosure, at most two of the at most seven nucleotides modified with the third modification are consecutively located. In some embodiments, in the antisense strand of the isolated oligonucleotide of the present disclosure, the at most two consecutively located of the at most seven nucleotides modified with the third modification are located at positions 2 and 3 from the first nucleotide at the 5′-terminus of the antisense strand. In some embodiments, in the antisense strand of the isolated oligonucleotide of the present disclosure, the at most two consecutively located of the at most seven nucleotides modified with the third modification are located at positions 5 and 6 from the first nucleotide at the 5′-terminus of the antisense strand. In some embodiments, in the antisense strand of the isolated oligonucleotide of the present disclosure, the at most two consecutively located of the at most seven nucleotides modified with the third modification are located at positions 6 and 7 from the first nucleotide at the 5′-terminus of the antisense strand.

In some embodiments, in the antisense strand of the isolated oligonucleotide of the present disclosure, at most three of the at most seven nucleotides modified with the third modification are consecutively located. In some embodiments, in the antisense strand of the isolated oligonucleotide of the present disclosure, the at most three consecutively located of the at most seven nucleotides modified with the third modification are located at positions 4, 5 and 6 from the first nucleotide at the 5′-terminus of the antisense strand.

In some embodiments, in the antisense strand of the isolated oligonucleotide of the present disclosure, three or four of the at most seven nucleotides modified with the third modification are located at positions selected from position 2, 3, 5, 6, 7, and 8 from the first nucleotide at the 5′-terminus of the antisense strand. In some embodiments, in the antisense strand of the isolated oligonucleotide of the present disclosure, three of the at most seven nucleotides modified with the third modification are located at positions 2, 5 and 6 from the first nucleotide at the 5′-terminus of the antisense strand. In some embodiments, in the antisense strand of the isolated oligonucleotide of the present disclosure, three of the at most seven nucleotides modified with the third modification are located at positions 2, 3 and 6 from the first nucleotide at the 5′-terminus of the antisense strand. In some embodiments, in the antisense strand of the isolated oligonucleotide of the present disclosure, three of the at most seven nucleotides modified with the third modification are located at positions 2, 6 and 7 from the first nucleotide at the 5′-terminus of the antisense strand. In some embodiments, in the antisense strand of the isolated oligonucleotide of the present disclosure, three of the at most seven nucleotides modified with the third modification are located at positions 2, 6 and 8 from the first nucleotide at the 5′-terminus of the antisense strand.

In some embodiments, in the antisense strand of the isolated oligonucleotide of the present disclosure, at least one of the at most seven nucleotides modified with the third modification is located at position 14 from the first nucleotide at the 5′-terminus of the antisense strand. In some embodiments, in the antisense strand of the isolated oligonucleotide of the present disclosure, two or three of the at most seven nucleotides modified with the third modification are located at positions selected from position 2, 3, 5, and 6 from the first nucleotide at the 5′-terminus of the antisense strand. In some embodiments, in the antisense strand of the isolated oligonucleotide of the present disclosure, three of the at most seven nucleotides modified with the third modification are located at positions selected from position 2, 3, 5, and 6 from the first nucleotide at the 5′-terminus of the antisense strand. In some embodiments, in the antisense strand of the isolated oligonucleotide of the present disclosure, two of the at most seven nucleotides modified with the third modification are located at positions 2 and 6 from the first nucleotide at the 5′-terminus of the antisense strand. In some embodiments, in the antisense strand of the isolated oligonucleotide of the present disclosure, two of the at most seven nucleotides modified with the third modification are located at positions 2 and 5 from the first nucleotide at the 5′-terminus of the antisense strand. In some embodiments, in the antisense strand of the isolated oligonucleotide of the present disclosure, two of the at most seven nucleotides modified with the third modification are located at positions 2 and 3 from the first nucleotide at the 5′-terminus of the antisense strand.

In some embodiments, in the antisense strand of the isolated oligonucleotide of the present disclosure, three of the at most seven nucleotides modified with the third modification are located at positions 2, 3 and 6 from the first nucleotide at the 5′-terminus of the antisense strand. In some embodiments, in the antisense strand of the isolated oligonucleotide of the present disclosure, three of the at most seven nucleotides modified with the third modification are located at positions 2, 3 and 5 from the first nucleotide at the 5′-terminus of the antisense strand. In some embodiments, in the antisense strand of the isolated oligonucleotide of the present disclosure, three of the at most seven nucleotides modified with the third modification are located at positions 2, 5 and 6 from the first nucleotide at the 5′-terminus of the antisense strand.

In some embodiments, in the antisense strand of the isolated oligonucleotide of the present disclosure, two of the at most seven nucleotides modified with the third modification are located at positions 8 and 14 from the nucleotide complementary to the first nucleotide at the 5′-terminus of the antisense strand.

In some embodiments, in the antisense strand of the isolated oligonucleotide of the present disclosure, one or two of the at most seven nucleotides modified with the third modification are located at positions selected from position 14 and 16 from the first nucleotide at the 5′-terminus of the antisense strand. In some embodiments, in the antisense strand of the isolated oligonucleotide of the present disclosure, two of the at most seven nucleotides modified with the third modification are located at positions 14 and 16 from the first nucleotide at the 5′-terminus of the antisense strand. In some embodiments, in the antisense strand of the isolated oligonucleotide of the present disclosure, the at most seven nucleotides are modified with 2′-F modification. In some embodiments, in the antisense strand of the isolated oligonucleotide of the present disclosure, one of the at most seven nucleotides modified with the third modification is located at position 14 from the first nucleotide at the 5′-terminus of the antisense strand. In some embodiments, in the antisense strand of the isolated oligonucleotide of the present disclosure, two of the at most seven nucleotides modified with the third modification is located at positions 14 and 16 from the first nucleotide at the 5′-terminus of the antisense strand.

In some embodiments, in the antisense strand of the isolated oligonucleotide of the present disclosure, the at most seven nucleotides modified with the third modification are located at positions selected from position 2, 3, 5, 6, 7, 8, 9, 14 and 16 from the first nucleotide at the 5′-terminus of the antisense strand. In some embodiments, in the antisense strand of the isolated oligonucleotide of the present disclosure, the at most seven nucleotides modified with the third modification are located at positions 2, 3, 6, 14 and 16 from the first nucleotide at the 5′-terminus of the antisense strand (e.g., SEQ ID NO: 15, SEQ ID NO: 45). In some embodiments, in the antisense strand of the isolated oligonucleotide of the present disclosure, the at most seven nucleotides modified with the third modification are located at positions 2, 6, 7, 14 and 16 from the first nucleotide at the 5′-terminus of the antisense strand (e.g., SEQ ID NO: 16, SEQ ID NO: 46). In some embodiments, in the antisense strand of the isolated oligonucleotide of the present disclosure, the at most seven nucleotides modified with the third modification are located at positions 2, 3, 6, 8, 9, 14 and 16 from the first nucleotide at the 5′-terminus of the antisense strand (e.g., SEQ ID NO: 22, SEQ ID NO: 39). In some embodiments, in the antisense strand of the isolated oligonucleotide of the present disclosure, the at most seven nucleotides modified with the third modification are located at positions 2, 3, 5, 8, 9, 14 and 16 from the first nucleotide at the 5′-terminus of the antisense strand (e.g., SEQ ID NO: 23, SEQ ID NO: 40). In some embodiments, in the antisense strand of the isolated oligonucleotide of the present disclosure, the at most seven nucleotides modified with the third modification are located at positions 2, 3, 5, 7, 9, 14 and 16 from the first nucleotide at the 5′-terminus of the antisense strand (e.g., SEQ ID NO: 24, SEQ ID NO: 41). In some embodiments, in the antisense strand of the isolated oligonucleotide of the present disclosure, the at most seven nucleotides modified with the third modification are located at positions 2, 3, 5, 7, 10, 14 and 16 from the first nucleotide at the 5′-terminus of the antisense strand (e.g., SEQ ID NO: 25, SEQ ID NO: 42).

In some embodiments, in the antisense strand of the isolated oligonucleotide of the present disclosure, the at most six nucleotides are modified with the third modification. In some embodiments, in the antisense strand of the isolated oligonucleotide of the present disclosure, the at most six nucleotides are modified with 2′-F modification.

In some embodiments, in the antisense strand of the isolated oligonucleotide of the present disclosure, at most five nucleotides are modified with the third modification. In some embodiments, in the antisense strand of the isolated oligonucleotide of the present disclosure, the at most five nucleotides are modified with 2′-F modification. In some embodiments, in the antisense strand of the isolated oligonucleotide of the present disclosure, the at most five nucleotides modified with the third modification are located at positions selected from positions 2, 3, 5, 6, 7, 8, 9, 14 and 16 from the first nucleotide at the 5′-terminus of the antisense strand.

In some embodiments, in the antisense strand of the isolated oligonucleotide of the present disclosure, the at most five nucleotides are modified with the third modification at positions 2, 3, 6, 14 and 16, from the nucleotide complementary to the first nucleotide at the 5′-terminus of the antisense strand. In some embodiments, in the antisense strand of the isolated oligonucleotide of the present disclosure, the at most five nucleotides are modified with the third modification at positions 2, 6, 7, 14 and 16, from the nucleotide complementary to the first nucleotide at the 5′-terminus of the antisense strand. In some embodiments, in the antisense strand of the isolated oligonucleotide of the present disclosure, the at most six nucleotides are modified with the third modification at positions 2, 3, 6, 8, 9 and 16, from the nucleotide complementary to the first nucleotide at the 5′-terminus of the antisense strand. In some embodiments, in the antisense strand of the isolated oligonucleotide of the present disclosure, the at most six nucleotides are modified with the third modification at positions 2, 3, 5, 8, 9 and 16, from the nucleotide complementary to the first nucleotide at the 5′-terminus of the antisense strand. In some embodiments, in the antisense strand of the isolated oligonucleotide of the present disclosure, the at most seven nucleotides are modified with the third modification at positions 2, 3, 5, 7, 9, 14 and 16, from the nucleotide complementary to the first nucleotide at the 5′-terminus of the antisense strand. In some embodiments, in the antisense strand of the isolated oligonucleotide of the present disclosure, the at most seven nucleotides are modified with the third modification at positions 2, 3, 5, 7, 10, 14 and 16, from the nucleotide complementary to the first nucleotide at the 5′-terminus of the antisense strand.

In some embodiments, in the antisense strand of the isolated oligonucleotide of the present disclosure, the at most five nucleotides modified with the third modification are located at positions 2, 5, 6, 14 and 16 from the first nucleotide at the 5′-terminus of the antisense strand. In some embodiments, in the antisense strand of the isolated oligonucleotide of the present disclosure, the at most five nucleotides modified with the third modification are located at positions 2, 3, 6, 14 and 16 from the first nucleotide at the 5′-terminus of the antisense strand. In some embodiments, in the antisense strand of the isolated oligonucleotide of the present disclosure, the at most five nucleotides modified with the third modification are located at positions 2, 6, 8, 14 and 16 from the first nucleotide at the 5′-terminus of the antisense strand. In some embodiments, in the antisense strand of the isolated oligonucleotide of the present disclosure, the at most five nucleotides modified with the third modification are located at positions 2, 6, 9, 14 and 16 from the first nucleotide at the 5′-terminus of the antisense strand.

In some embodiments, in the antisense strand of the isolated oligonucleotide of the present disclosure, at most four nucleotides are modified with the third modification. In some embodiments, in the antisense strand of the isolated oligonucleotide of the present disclosure, at most four nucleotides are modified with 2′-F modification. In some embodiments, in the antisense strand of the isolated oligonucleotide of the present disclosure, the at most four nucleotides modified with the third modification are located at positions selected from positions 2, 3, 5, 6, 7, 8, 9, 14 and 16 from the first nucleotide at the 5′-terminus of the antisense strand. In some embodiments, in the antisense strand of the isolated oligonucleotide of the present disclosure, the at most four nucleotides modified with the third modification are located at positions 2, 6, 14 and 16 from the first nucleotide at the 5′-terminus of the antisense strand. In some embodiments, in the antisense strand of the isolated oligonucleotide of the present disclosure, the at most four nucleotides modified with the third modification are located at positions 2, 5, 14 and 16 from the first nucleotide at the 5′-terminus of the antisense strand.

In some embodiments, in the antisense strand of the isolated oligonucleotide of the present disclosure, at most three nucleotides are modified with the third modification. In some embodiments, in the antisense strand of the isolated oligonucleotide of the present disclosure, the at least three nucleotides are modified with 2′-F modification. In some embodiments, in the antisense strand of the isolated oligonucleotide of the present disclosure, the at most three nucleotides modified with the third modification are located at positions selected from positions 2, 3, 5, 6, 7, 8, 9, 14 and 16 from the first nucleotide at the 5′-terminus of the antisense strand. In some embodiments, in the antisense strand of the isolated oligonucleotide of the present disclosure, the at most three nucleotides modified with the third modification are located at positions 2, 6 and 14 from the first nucleotide at the 5′-terminus of the antisense strand.

In some embodiments, in the antisense strand of the isolated oligonucleotide of the present disclosure, the antisense strand comprises nucleotides modified with 2′-F modification, and nucleotides modified with 2′-O-methyl modification, according to the formula: 3′ (M)ₐ(F)_{b}(M)_{c}(F)_{d}(M)ₑ(F) (M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and each of a, b, c, d, e, f, g, h, i, j, k, l, m, n and o is any one of 0-16, and indicates the number of consecutive nucleotides modified with the modification, and wherein the antisense strand is any one of:

1)
(SEQ ID NO: 14)
3′(M)₀(F)₀(M)₀(F)₀(M)₀(F)₀(M)₇(F)₁(M)₁(F)₁(M)₇(F)₂(M)₂(F)₁(M)₁ 5′;
2)
(SEQ ID NO: 15)
3′(M)₀(F)₀(M)₀(F)₀(M)₀(F)₀(M)₇(F)₁(M)₁(F)₁(M)₇(F)₁(M)₂(F)₂(M)₁ 5′;
3)
(SEQ ID NO: 16)
3′(M)₀(F)₀(M)₀(F)₀(M)₀(F)₀(M)₇(F)₁(M)₁(F)₁(M)₆(F)₂(M)₃(F)₁(M)₁ 5′;
4)
(SEQ ID NO: 17)
3′(M)₀(F)₀(M)₀(F)₀(M)₇(F)₁(M)₁(F)₁(M)₅(F)₁(M)₁(F)₁(M)₃(F)₁(M)₁ 5′;
5)
(SEQ ID NO: 18)
3′(M)₀(F)₀(M)₀(F)₀(M)₇(F)₁(M)₁(F)₁(M)₄(F)₁(M)₂(F)₁(M)₃(F)₁(M)₁ 5′;
6)
(SEQ ID NO: 20)
3′(M)₀(F)₀(M)₀(F)₀(M)₀(F)₀(M)₇(F)₁(M)₁(F)₁(M)₈(F)₁(M)₂(F)₁(M)₁ 5′;
7)
(SEQ ID NO: 21)
3′(M)₀(F)₀(M)₀(F)₀(M)₀(F)₀(M)₀(F)₀(M)₉(F)₁(M)₇(F)₁(M)₃(F)₁(M)₁ 5′;
8)
(SEQ ID NO: 22)
3′(M)₀(F)₀(M)₀(F)₀(M)₇(F)₁(M)₁(F)₁(M)₄(F)₂(M)₁(F)₁(M)₂(F)₂(M)₁ 5′;
9)
(SEQ ID NO: 23)
3′(M)₀(F)₀(M)₀(F)₀(M)₇(F)₁(M)₁(F)₁(M)₄(F)₂(M)₂(F)₁(M)₁(F)₂(M)₁ 5′;
10)
(SEQ ID NO: 24)
3′(M)₀(F)₀(M)₀(F)₀(M)₇(F)₁(M)₁(F)₁(M)₄(F)₁(M)₁(F)₁(M)₁(F)₂(M)₁ 5′;
11)
(SEQ ID NO: 25)
3′(M)₀(F)₀(M)₀(F)₀(M)₇(F)₁(M)₁(F)₁(M)₃(F)₁(M)₂(F)₁(M)₁(F)₁(M)₁(F)₂(M)₁ 5′;
12)
(SEQ ID NO: 27)
3′(M)₀(F)₀(M)₄(F)₁(M)₁(F)₁(M)₁(F)₁(M)₃(F)₁(M)₁(F)₁(M)₁(F)₃(M)₁(F)₁(M)₁ 5′;
13)
(SEQ ID NO: 30)
3′(M)₀(F)₀(M)₀(F)₀(M)₀(F)₀(M)₆(F)₁(M)₁(F)₁(M)₇(F)₁(M)₃(F)₁(M)₁ 5′;
14)
(SEQ ID NO: 50)
3′(M)₀(F)₀(M)₀(F)₀(M)₀(F)₀(M)₆(F)₁(M)₁(F)₁(M)₇(F)₂(M)₂(F)₁(M)₁ 5′;
15)
(SEQ ID NO: 45)
3′(M)₀(F)₀(M)₀(F)₀(M)₀(F)₀(M)₆(F)₁(M)₁(F)₁(M)₇(F)₁(M)₂(F)₂(M)₁ 5′;
16)
(SEQ ID NO: 46)
3′(M)₀(F)₀(M)₀(F)₀(M)₀(F)₀(M)₆(F)₁(M)₁(F)₁(M)₆(F)₂(M)₃(F)₁(M)₁ 5′;
17)
(SEQ ID NO: 47)
3′(M)₀(F)₀(M)₀(F)₀(M)₆(F)₁(M)₁(F)₁(M)₅(F)₁(M)₁(F)₁(M)₃(F)₁(M)₁ 5′;
18)
(SEQ ID NO: 48)
3′(M)₀(F)₀(M)₀(F)₀(M)₆(F)₁(M)₁(F)₁(M)₄(F)₁(M)₂(F)₁(M)₃(F)₁(M)₁ 5′;
19)
(SEQ ID NO: 49)
3′(M)₀(F)₀(M)₀(F)₀(M)₆(F)₁(M)₁(F)₁(M)₄(F)₂(M)₁(F)₁(M)₃(F)₁(M)₁ 5′;
20)
(SEQ ID NO: 37)
3′(M)₀(F)₀(M)₀(F)₀(M)₀(F)₀(M)₆(F)₁(M)₁(F)₁(M)₈(F)₁(M)₂(F)₁(M)₁ 5′;
21)
(SEQ ID NO: 38)
3′(M)₀(F)₀(M)₀(F)₀(M)₀(F)₀(M)₀(F)₀(M)₈(F)₁(M)₇(F)₁(M)₃(F)₁(M)₁ 5′;
22)
(SEQ ID NO: 39)
3′(M)₀(F)₀(M)₀(F)₀(M)₆(F)₁(M)₁(F)₁(M)₄(F)₂(M)₁(F)₁(M)₂(F)₂(M)₁ 5′;
23)
(SEQ ID NO: 40)
3′(M)₀(F)₀(M)₀(F)₀(M)₆(F)₁(M)₁(F)₁(M)₄(F)₂(M)₂(F)₁(M)₁(F)₂(M)₁ 5′;
24)
(SEQ ID NO: 41)
3′(M)₀(F)₀(M)₆(F)₁(M)₁(F)₁(M)₄(F)₁(M)₁(F)₁(M)₁(F)₁(M)₁(F)₂(M)₁ 5′;
and
25)
(SEQ ID NO: 42)
3′(M)₀(F)₀(M)₆(F)₁(M)₁(F)₁(M)₃(F)₁(M)₂(F)₁(M)₁(F)₁(M)₁(F)₂(M)₁ 5′.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₀(F)₀(M)₇(F)₁(M)₁(F)₁(M)₇(F)₂(M)₂(F)₁(M)₁ 5′ (SEQ ID NO: 14), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₀(F)₀(M)₇(F)₁(M)₁(F)₁(M)₇(F)₁(M)₂(F)₂(M)₁ 5′ (SEQ ID NO: 15), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₀(F)₀(M)₇(F)₁(M)₁(F)₁(M)₆(F)₂(M)₃(F)₁(M)₁ 5′ (SEQ ID NO: 16), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₇(F)₁(M)₁(F)₁(M)₅(F)₁(M)₁(F)₁(M)₃(F)₁(M)₁ 5′ (SEQ ID NO: 17), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₇(F)₁(M)₁(F)₁(M)₄(F)₁(M)₂(F)₁(M)₃(F)₁(M)₁ 5′ (SEQ ID NO: 18), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₀(F)₀(M)₇(F)₁(M)₁(F)₁(M)₅(F)₁(M)₂(F)₁(M)₁ 5′ (SEQ ID NO: 20), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₀(F)₀(M)₀(F)₀(M)₉(F)₁(M)₇(F)₁(M)₃(F)₁(M)₁ 5′ (SEQ ID NO: 21), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀ (M)₀(F)₀(M)₇(F)₁(M)₁(F)₁(M)₄(F)₂(M)₁(F)₁(M)₂(F)₂(M)₁ 5′ (SEQ ID NO: 22), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₇(F)₁(M)₁(F)₁(M)₄(F)₂(M)₂(F)₁(M)₁(F)₂(M)₁ 5′ (SEQ ID NO: 23), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₇(F)₁(M)₁(F)₁(M)₄(F)₁(M)₁(F)₁(M)₁(F)₂(M)₁ 5′ (SEQ ID NO: 24), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₇(F)₁(M)₁(F)₁(M)₃(F)₁(M)₂(F)₁(M)₁(F)₁(M)₁(F)₂(M)₁ 5′ (SEQ ID NO: 25), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₄(F)₁(M)₁(F)₁(M)₁(F)₁(M)₃(F)₁(M)₁(F)₁(M)₁(F)₃(M)₁(F)₁(M)₁ 5′ (SEQ ID NO: 27), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₇(F)₁(M)₃(F)₁(M)₁ 5′ (SEQ ID NO: 30), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₇(F)₂(M)₂(F)₁(M)₁ 5′ (SEQ ID NO: 50), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₇(F)₁(M)₂(F)₂(M)₁ 5′ (SEQ ID NO: 45), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₆(F)₂(M)₃(F)₁(M)₁ 5′ (SEQ ID NO: 46), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₆(F)₁(M)₁(F)₁(M)₅(F)₁(M)₁(F)₁(M)₃(F)₁(M)₁ 5′ (SEQ ID NO: 47), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₆(F)₁(M)₁(F)₁(M)₄(F)₁(M)₂(F)₁(M)₃(F)₁(M)₁ 5′ (SEQ ID NO: 48), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₆(F)₁(M)₁(F)₁(M)₄(F)₂(M)₁(F)₁(M)₃(F)₁(M)₁ 5′ (SEQ ID NO: 49), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₈(F)₁(M)₂(F)₁(M)₁ 5′ (SEQ ID NO: 37), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₀(F)₀(M)₀(F)₀(M)₈(F)₁(M)₇(F)₁(M)₃(F)₁(M)₁ 5′ (SEQ ID NO: 38), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₆(F)₁(M)₁(F)₁(M)₄(F)₂(M)₁(F)₁(M)₂(F)₂(M)₁ 5′ (SEQ ID NO: 39), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₀(F)₀(M)₆(F)₁(M)₁(F)₁(M)₄(F)₂(M)₂(F)₁(M)₁(F)₂(M)₁ 5′ (SEQ ID NO: 40), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₆(F)₁(M)₁(F)₁(M)₄(F)₁(M)₁(F)₁(M)₁(F)₁(M)₁(F)₂(M)₁ 5′ (SEQ ID NO: 41), wherein M is 2′-O-methyl modified nucleotide, F is 2′-F modified nucleotide.

In some embodiments of the isolated oligonucleotide of the present disclosure, the antisense strand comprises nucleotides modified with 2′-F modification, and nucleotides modified with 2′-O-methyl modification, according to the formula: 3′(M)₀(F)₀(M)₆(F)₁(M)₁(F)₁(M)₃(F)₁(M)₂(F)₁(M)₁(F)₁(M)₁(F)₂(M)₁ 5′ (SEQ ID NO: 42), wherein M is 2′-O-methyl modified nucleotide, F is 2′-F modified nucleotide.

In some embodiments of the isolated oligonucleotide of the present disclosure, the sense strand comprises nucleotides modified with the first modification located at positions 11, 12, 13 and 15 from the nucleotide complementary to the first nucleotide at the 5′-terminus of the sense strand, and the antisense strand comprises nucleotides modified with the first modification located at positions 2, 6, 14 and 16 from the nucleotide complementary to the first nucleotide at the 5′-terminus of the antisense strand.

In some embodiments of the isolated oligonucleotide of the present disclosure, the sense strand comprises nucleotides modified with the first modification located at positions 11, 12 and 13 from the nucleotide complementary to the first nucleotide at the 5′-terminus of the sense strand, and the antisense strand comprises nucleotides modified with the first modification located at positions 2, 6, 14 and 16 from the nucleotide complementary to the first nucleotide at the 5′-terminus of the antisense strand.

In some embodiments of the isolated oligonucleotide of the present disclosure, the sense strand comprises nucleotides modified with the first modification located at positions 11, 12, 13 and 14 from the nucleotide complementary to the first nucleotide at the 5′-terminus of the sense strand, and the antisense strand comprises nucleotides modified with the first modification located at positions 2, 6, 14 and 16 from the nucleotide complementary to the first nucleotide at the 5′-terminus of the antisense strand.

In some embodiments of the isolated oligonucleotide of the present disclosure, the sense strand comprises nucleotides modified with the first modification located at positions 10, 11, 12 and 13 from the nucleotide complementary to the first nucleotide at the 5′-terminus of the sense strand, and the antisense strand comprises nucleotides modified with the first modification located at positions 2, 6, 14 and 16 from the nucleotide complementary to the first nucleotide at the 5′-terminus of the antisense strand.

In some embodiments of the isolated oligonucleotide of the present disclosure, the sense strand comprises nucleotides modified with the first modification located at positions 10, 11, 12, 13 and 14 from the nucleotide complementary to the first nucleotide at the 5′-terminus of the sense strand, and the antisense strand comprises nucleotides modified with the first modification located at positions 2, 6, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 12, 13 and 15 from the nucleotide complementary to the first nucleotide at the 5′-terminus of the sense strand, and the antisense strand comprises nucleotides modified with the first modification located at positions 2, 6, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 10, 12, 13 and 15 from the nucleotide complementary to the first nucleotide at the 5′-terminus of the sense strand, and the antisense strand comprises nucleotides modified with the first modification located at positions 2, 6, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 10, 11, 12, 13 and 15 from the nucleotide complementary to the first nucleotide at the 5′-terminus of the sense strand, and the antisense strand comprises nucleotides modified with the first modification located at positions 2, 6, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 11, 12, 13 and 15 from the nucleotide complementary to the first nucleotide at the 5′-terminus of the sense strand, and the antisense strand comprises nucleotides modified with the first modification located at positions 2, 5, 6, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 11, 12, 13 and 15 from the nucleotide complementary to the first nucleotide at the 5′-terminus of the sense strand, and the antisense strand comprises nucleotides modified with the first modification located at positions 2, 5, 6, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 11, 12, 13 and 15 from the nucleotide complementary to the first nucleotide at the 5′-terminus of the sense strand, and the antisense strand comprises nucleotides modified with the first modification located at positions 2, 3, 6, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 11, 12, 13 and 15 from the nucleotide complementary to the first nucleotide at the 5′-terminus of the sense strand, and the antisense strand comprises nucleotides modified with the first modification located at positions 2, 6, 7, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 11, 12, 13 and 15 from the nucleotide complementary to the first nucleotide at the 5′-terminus of the sense strand, and the antisense strand comprises nucleotides modified with the first modification located at positions 2, 6, 8, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 11, 12, 13 and 15 from the nucleotide complementary to the first nucleotide at the 5′-terminus of the sense strand, and the antisense strand comprises nucleotides modified with the first modification located at positions 2, 6, 9, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 11, 12, 13 and 15 from the nucleotide complementary to the first nucleotide at the 5′-terminus of the sense strand, and the antisense strand comprises nucleotides modified with the first modification located at positions 2, 6, 8, 9, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 11, 12, 13 and 15 from the nucleotide complementary to the first nucleotide at the 5′-terminus of the sense strand, and the antisense strand comprises nucleotides modified with the first modification located at positions 2, 5, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 11, 12, 13 and 15 from the nucleotide complementary to the first nucleotide at the 5′-terminus of the sense strand, and the antisense strand comprises nucleotides modified with the first modification located at positions 2, 6 and 14 from the nucleotide complementary to the first nucleotide at the 5′-terminus of the antisense strand.

In some embodiments of the isolated oligonucleotide of the present disclosure, the sense strand comprises nucleotides modified with the modification located at positions 10, 11, 12, 13 and 15 from the nucleotide complementary to the first nucleotide at the 5′-terminus of the sense strand, and the antisense strand comprises nucleotides modified with the first modification located at positions 2, 3, 6, 8, 9, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 10, 11, 12 and 13 from the nucleotide complementary to the first nucleotide at the 5′-terminus of the sense strand, and the antisense strand comprises nucleotides modified with the first modification located at positions 2, 3, 6, 8, 9, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 10, 11, 12, 13 and 15 from the nucleotide complementary to the first nucleotide at the 5′-terminus of the sense strand, and the antisense strand comprises nucleotides modified with the first modification located at positions 2, 3, 5, 8, 9, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 10, 11, 12, 13 and 15 from the nucleotide complementary to the first nucleotide at the 5′-terminus of the sense strand, and the antisense strand comprises nucleotides modified with the first modification located at positions 2, 3, 5, 7, 9, 14 and 16 from the nucleotide complementary to the first nucleotide at the 5′-terminus of the antisense strand.

In some embodiments of the isolated oligonucleotide of the present disclosure, the sense strand comprises nucleotides modified with the modification located at positions 10, 11, 12, 13 and 15 from the nucleotide complementary to the first nucleotide at the 5′-terminus of the sense strand, and the antisense strand comprises nucleotides modified with the first modification located at positions 2, 3, 5, 7, 10, 14 and 16 from the nucleotide complementary to the first nucleotide at the 5′-terminus of the antisense strand.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₆(F)₁(M)₁(F)₄(M)₉ 3′; and the antisense strand comprises nucleotides modified with 2′-F modification, and nucleotides modified with 2′-O-methyl modification, according to the formula: 3′(M)₀(F)₀ (M)₀(F)₀(M)₇(F)₁(M)₁(F)₁(M)₄(F)₂(M)₁(F)₁(M)₂(F)₂(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₀(F)₀(M)₈(F)₄(M)₉ 3′; and the antisense strand comprises nucleotides modified with 2′-F modification, and nucleotides modified with 2′-O-methyl modification, according to the formula: 3′(M)₀(F)₀ (M)₀(F)₀(M)₇(F)₁(M)₁(F)₁(M)₄(F)₂(M)₁(F)₁(M)₂(F)₂(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₆(F)₁(M)₁(F)₄(M)₉ 3′; and the antisense strand comprises nucleotides modified with 2′-F modification, and nucleotides modified with 2′-O-methyl modification, according to the formula: 3′(M)₀(F)₀(M)₀(F)₀(M)₇(F)₁(M)₁(F)₁(M)₄(F)₂(M)₂(F)₁(M)₁(F)₂(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₆(F)₁(M)₁(F)₄(M)₉ 3′; and the antisense strand comprises nucleotides modified with 2′-F modification, and nucleotides modified with 2′-O-methyl modification, according to the formula: 3′(M)₀(F)₀(M)₀(F)₀(M)₇(F)₁(M)₁(F)₁(M)₄(F)₁(M)₁(F)₁(M)₁(F)₂(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₆(F)₁(M)₁(F)₄(M)₉ 3′; and the antisense strand comprises nucleotides modified with 2′-F modification, and nucleotides modified with 2′-O-methyl modification, according to the formula: 3′(M)₀(F)₀(M)₀(F)₀(M)₇(F)₁(M)₁(F)₁(M)₄(F)₁(M)₁(F)₁(M)₁(F)₂(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₃(M)₁₀ 3′; and the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₇(F)₁(M)₃(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₀(F)₀(M)₇(F)₃(M)₁₀ 3′; and the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₇(F)₁(M)₃(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₀(F)₀(M)₆(F)₄(M)₁₀ 3′; and the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₇(F)₁(M)₃(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₀(F)₀(M)₇(F)₄(M)₉ 3′; and the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₇(F)₁(M)₃(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₀(F)₀(M)₆(F)₅(M)₉ 3′; and the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₇(F)₁(M)₃(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₂(M)₁₁ 3′; and the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₇(F)₁(M)₃(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₅(F)₁(M)₁(F)₂(M)₁(F)₁(M)₉ 3′; and the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₇(F)₁(M)₃(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₄(M)₉ 3′; and the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₇(F)₁(M)₃(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₃(M)₁₀ 3′; and the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₇(F)₁(M)₃(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₃(M)₁₀ 3′; and the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₇(F)₂(M)₂(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₃(M)₁₀ 3′; and the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₇(F)₁(M)₂(F)₂(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₃(M)₁₀ 3′; and the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₆(F)₂(M)₃(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₃(M)₁₀ 3′; and the antisense strand comprises nucleotides modified with 2′-F modification, and nucleotides modified with 2′-O-methyl modification, according to the formula: 3′(M)₀(F)₀(M)₀(F)₀(M)₆(F)₁(M)₁(F)₁(M)₅(F)₁(M)₁(F)₁(M)₃(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₃(M)₁₀ 3′; and the antisense strand comprises nucleotides modified with 2′-F modification, and nucleotides modified with 2′-O-methyl modification, according to the formula: 3′(M)₀(F)₀(M)₀(F)₀(M)₆(F)₁(M)₁(F)₁(M)₄(F)₁(M)₂(F)₁(M)₃(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₃(M)₁₀ 3′; and the antisense strand comprises nucleotides modified with 2′-F modification, and nucleotides modified with 2′-O-methyl modification, according to the formula: 3′(M)₀(F)₀(M)₀(F)₀(M)₆(F)₁(M)₁(F)₁(M)₄(F)₂(M)₁(F)₁(M)₃(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₃(M)₁₀ 3′; and the antisense strand comprises nucleotides modified with 2′-F modification, and nucleotides modified with 2′-O-methyl modification, according to the formula: 3′(M)₀(F)₀(M)₀(F)₀(M)₀(F)₀(M)₆(F)₁(M)₁(F)₁(M)₈(F)₁(M)₂(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₃(M)₁₀ 3′; and the antisense strand comprises nucleotides modified with 2′-F modification, and nucleotides modified with 2′-O-methyl modification, according to the formula: 3′(M)₀(F)₀(M)₀(F)₀(M)₀(F)₀(M)₀(F)₀(M)₈(F)₁(M)₇(F)₁(M)₃(F)₁(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₄(M)₉ 3′; and the antisense strand comprises nucleotides modified with 2′-F modification, and nucleotides modified with 2′-O-methyl modification, according to the formula: 3′(M)₀(F)₀(M)₀(F)₀(M)₆(F)₁(M)₁(F)₁(M)₄(F)₂(M)₁(F)₁(M)₂(F)₂(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₀(F)₀(M)₇(F)₄(M)₉ 3′; and the antisense strand comprises nucleotides modified with 2′-F modification, and nucleotides modified with 2′-O-methyl modification, according to the formula: 3′(M)₀(F)₀(M)₀(F)₀(M)₆(F)₁(M)₁(F)₁(M)₄(F)₂(M)₁(F)₁(M)₂(F)₂(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₄(M)₉ 3′; and the antisense strand comprises nucleotides modified with 2′-F modification, and nucleotides modified with 2′-O-methyl modification, according to the formula: 3′(M)₀(F)₀(M)₀(F)₀(M)₆(F)₁(M)₁(F)₁(M)₄(F)₂(M)₂(F)₁(M)₁(F)₂(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₄(M)₉ 3′; and the antisense strand comprises nucleotides modified with 2′-F modification, and nucleotides modified with 2′-O-methyl modification, according to the formula: 3′(M)₀(F)₀(M)₆(F)₁(M)₁(F)₁(M)₄(F)₁(M)₁(F)₁(M)₁(F)₁(M)₁(F)₂(M)₁ 5′, wherein M is 2′-O-methyl modified nucleotide, F is 2′-F modified nucleotide.

In some embodiments of the isolated oligonucleotide of the present disclosure, the sense strand comprises nucleotides modified with 2′-F modification, and nucleotides modified with 2′-O-methyl modification, according to the formula: 5′(M)₀(F)₀(M)₅(F)₁(M)₁(F)₄(M)₉ 3′; and the antisense strand comprises nucleotides modified with 2′-F modification, and nucleotides modified with 2′-O-methyl modification, according to the formula: 3′(M)₀(F)₀(M)₆(F)₁(M)₁(F)₁(M)₃(F)₁(M)₂(F)₁(M)₁(F)₁(M)₁(F)₂(M)₁ 5′, wherein M is 2′-O-methyl modified nucleotide, F is 2′-F modified nucleotide.

In some embodiments, the X1 nucleotides of the sense strand of the isolated oligonucleotide of the present disclosure, is 18-21 and the X2 nucleotides of the antisense strand of the isolated oligonucleotide of the present disclosure is 20-23. In some embodiments, the X1 nucleotides of the sense strand of the isolated oligonucleotide of the present disclosure, is 20 or 21 and the X2 nucleotides of the antisense strand of the isolated oligonucleotide of the present disclosure is 22 or 23. In some embodiments, the X2 nucleotides of the antisense strand of the isolated oligonucleotide of the present disclosure equals the X1 nucleotides of the sense strand of the isolated oligonucleotide of the present disclosure plus 2. In some embodiments, the X1 nucleotides of the sense strand of the isolated oligonucleotide of the present disclosure, is 21 and the X2 nucleotides of the antisense strand of the isolated oligonucleotide of the present disclosure is 23. In some embodiments, the X1 nucleotides of the sense strand of the isolated oligonucleotide of the present disclosure, is 20 and the X2 nucleotides of the antisense strand of the isolated oligonucleotide of the present disclosure is 22.

In some embodiments, the sense strand of the isolated oligonucleotide of the present disclosure comprises at least one nucleotide having a modified phosphate backbone. In some embodiments, the antisense strand of the isolated oligonucleotide of the present disclosure comprises at least one nucleotide having a modified phosphate backbone. In some embodiments, in the sense strand or the antisense strand or both sense and antisense strands of the isolated oligonucleotide of the present disclosure, the modified phosphate backbone comprises a modified phosphodiester bond. In some embodiments, the modified phosphodiester bond is modified by replacing one or more oxygen atoms with a moiety, wherein the moiety is bonded to the phosphorus atom in the phosphodiester bond with a carbon, nitrogen, or sulfur atom in the moiety, or by forming a 2′-5′ linkage. In some embodiments, the modified phosphodiester bond comprises phosphorothioate, phosphorodithioate, methylphosphonate, phosphoramidate diester, mesyl phosphoramidate, or phosphonoacetate.

In some embodiments, the isolated oligonucleotide of the present disclosure comprises one or more non-natural base-containing nucleotide, a locked nucleotide, or an abasic nucleotide. In some embodiments, the isolated oligonucleotide of the present disclosure, the terminal nucleotide at the 5′ end comprises a phosphate mimic. In some embodiments, the 5′-phosphate mimic is ethylphosphonate, vinylphosphonate or an analog thereof.

In some embodiments, the antisense strand of the isolated oligonucleotide of the present disclosure comprises at least two single-stranded nucleotides at the 3′-terminus. In some embodiments, the antisense strand of the isolated oligonucleotide of the present disclosure comprises two single-stranded nucleotides at the 3′-terminus.

### Targeting Ligand

In some embodiments, in the sense strand or the antisense strand or both of the isolated oligonucleotide of the present disclosure, a terminal or internal nucleotide is linked to a targeting ligand. In some embodiments, the targeting ligand is attached to one or more nucleotides at the 5′ end of the sense strand of the isolated oligonucleotide of the present disclosure. In some embodiments, the targeting ligand is attached to one or more nucleotides at the 3′ end of the sense strand of the isolated oligonucleotide of the present disclosure. In some embodiments, the targeting ligand is attached to one or more nucleotides at the 5′ end of the antisense strand of the isolated oligonucleotide of the present disclosure. In some embodiments, the targeting ligand is attached to one or more nucleotides at the 3′ end of the antisense strand of the isolated oligonucleotide of the present disclosure. In some embodiments, the targeting ligand is attached to one or more nucleotides of the at least two single-stranded nucleotides at the 3′-terminus of the antisense strand of the isolated oligonucleotide of the present disclosure.

In some embodiments, the targeting ligand is selected from one or more of a carbohydrate, a peptide, a lipid, an antibody or a fragment thereof, an aptamer, an albumin, a fibrinogen, and a folate. In some embodiments, the targeting ligand binds to a surface protein on a cell expressing a target mRNA of the isolated oligonucleotide of the present disclosure. In some embodiments, the targeting ligand mediates entry of the isolated oligonucleotide of the present disclosure, into a cell expressing a target mRNA of the isolated oligonucleotide of the present disclosure.

In some embodiments, the targeting ligand is a therapeutic ligand. In some embodiments, the targeting ligand is a therapeutic antibody.

In some embodiments, the targeting ligand is attached to the isolated oligonucleotide of the present disclosure by a linker. In some embodiments, the linker is any one or a protein, a DNA, an RNA or a chemical compound. In some embodiments, the isolated oligonucleotide, the linker and the targeting ligand, of the present disclosure form a scaffold. As used herein, the term “scaffold” refers to a compound or complex that comprises a linker of the present disclosure, wherein the linker is covalently attached to either a ligand or an isolated oligonucleotide or both.

In some embodiments, the isolated oligonucleotide, the linker and the targeting ligand, of the present disclosure form a conjugate. As used herein, the term “conjugate” refers to a compound or complex that comprises an isolated oligonucleotide being covalently attached to a ligand via a linker of the present disclosure.

As used herein, the term “targeting ligand” or “ligand” refers to a moiety that, when being covalently attached to a Nucleic Acid Agent (e.g., an oligonucleotide), is capable of mediating its entry into, or facilitating or allowing its delivery to, a target site (e.g., a target cell or tissue). In some embodiments, the targeting ligand comprises a sugar ligand moiety (e.g., N-acetylgalactosamine (GalNAc)) which may direct uptake of an oligonucleotide into the liver.

In some embodiments, the targeting ligand binds to the asialoglycoprotein receptor (ASGPR). In some embodiments, the targeting ligand binds to (e.g., through ASGPR) the liver, such as the parenchymal cells of the liver.

Suitable targeting ligands include, but are not limited to, the ligands disclosed in Winkler (Ther. Deliv., 2013, 4(7): 791-809), PCT Patent Appl'n Pub. Nos. WO/2016/100401, WO/2012/089352, and WO/2009/082607, and U.S. Patent Appl'n Pub. Nos. 2009/0239814, 2012/0136042, 2013/0158824, and 2009/0247608, each of which is incorporated by reference.

In some embodiments, the targeting ligand comprises a carbohydrate moiety.

As used herein, “carbohydrate moiety” refers to a moiety which comprises one or more monosaccharide units each having at least six carbon atoms (which may be linear, branched or cyclic), with an oxygen, nitrogen or sulfur atom bonded to each carbon atom. In some embodiments, the carbohydrate moiety comprises a monosaccharide, a disaccharide, a trisaccharide, or a tetrasaccharide. In some embodiments, the carbohydrate moiety comprises an oligosaccharide containing from about 4-9 monosaccharide units. In some embodiments, the carbohydrate moiety comprises a polysaccharide (e.g., a starch, a glycogen, a cellulose, or a polysaccharide gum).

In some embodiments, the carbohydrate moiety comprises a monosaccharide, a disaccharide, a trisaccharide, or a tetrasaccharide. In some embodiments, the carbohydrate moiety comprises an oligosaccharide (e.g., containing from about four to about nine monosaccharide units). In some embodiments, the carbohydrate moiety comprises a polysaccharide (e.g., a starch, a glycogen, a cellulose, or a polysaccharide gum).

In some embodiments, the ligand is capable of binding to a human asialoglycoprotein receptor (ASGPR), e.g., human asialoglycoprotein receptor 2 (ASGPR2).

In some embodiments, the carbohydrate moiety comprises a sugar (e.g., one, two, or three sugar). In some embodiments, the carbohydrate moiety comprises galactose or a derivative thereof (e.g., one, two, or three galactose or the derivative thereof). In some embodiments, the carbohydrate moiety comprises N-acetylgalactosamine or a derivative thereof (e.g., one, two, or three N-acetylgalactosamine or the derivative thereof). In some embodiments, the carbohydrate moiety comprises N-acetyl-D-galactosylamine or a derivative thereof (e.g., one, two, or three N-acetyl-D-galactosylamine or the derivative thereof).

In some embodiments, the carbohydrate moiety comprises N-acetylgalactosamine (e.g., one, two, or three N-acetylgalactosamine). In some embodiments, the carbohydrate moiety comprises N-acetyl-D-galactosylamine (e.g., one, two, or three N-acetyl-D-galactosylamine).

In some embodiments, the carbohydrate moiety comprises mannose or a derivative thereof (e.g., mannose-6-phosphate). In some embodiments, the carbohydrate moiety further comprises a linking moiety that connects the one or more sugar (e.g., N-acetyl-D-galactosylamine) with the Linker.

In some embodiments the Linker comprises thioether (e.g., thiosuccinimide, or the hydrolysis analogue thereof), disulfide, triazole, phosphorothioate, phosphodiester, ester, amide, or any combination thereof. In some embodiments, the Linker is a triantennary linking moiety. Suitable targeting ligands include, but are not limited to, the ligands disclosed in PCT Appl'n Pub. Nos. WO/2015/006740, WO/2016/100401, WO/2017/214112, WO/2018/039364, and WO/2018/045317, each of which is incorporated herein by reference.

In some embodiments, the targeting ligand comprises a lipid or a lipid moiety (e.g., one, two, or three lipid moiety). In some embodiments the lipid moiety comprises (e.g., one, two, of three of) C8-C24 fatty acid, cholesterol, vitamin, sterol, phospholipid, or any combination thereof.

In some embodiments, the targeting ligand comprises a peptide or a peptide moiety (e.g., one, two, or three peptide moiety). In some embodiments, the peptide moiety comprises (e.g., one, two, or three of) integrin, insulin, glucagon-like peptide, or any combination thereof. In some embodiments, the targeting ligand comprises an antibody or an antibody moiety (e.g., transferrin). In some embodiments, the targeting ligand comprises one, two, or three antibody moieties (e.g., transferrin).

In some embodiments, the targeting ligand comprises an oligonucleotide (e.g., aptamer or CpG). In some embodiments, the targeting ligand comprises one, two, or three oligonucleotides (e.g., aptamer or CpG).

In some embodiments, the ligand comprises: one, two, or three sugar (e.g., N-acetyl-D-galactosylamine); one, two, or three lipid moieties; one, two, or three peptide moieties; one, two, or three antibody moieties; one, two, or three oligonucleotides; or any combination thereof.

In some embodiments, the Linker is attached to the isolated oligonucleotide of the present disclosure, via a phosphate group, or an analog of a phosphate group, in the isolated oligonucleotide.

### Immunostimulatory Oligonucleotides

In some embodiments, the isolated oligonucleotide of the present disclosure linked to a lipid, carbohydrate or peptide, that may be immunostimulatory, including immunostimulatory oligonucleotides (ISS; single- or double-stranded) capable of inducing an immune response when administered to a subject, which may be a mammal or other patient. ISS include, e.g., certain palindromes leading to hairpin secondary structures (see Yamamoto S., et al. (1992) J. Immunol. 148: 4072-4076, which is incorporated by reference in its entirety), or CpG motifs, as well as other known ISS features (such as multi-G domains, see WO 96/11266, which is incorporated by reference in its entirety).

The immune response may be an innate or an adaptive immune response. The immune system is divided into a more innate immune system, and acquired adaptive immune system of vertebrates, the latter of which is further divided into humoral cellular components. In some embodiments, the immune response may be mucosal.

In some embodiments, an immunostimulatory isolated oligonucleotide is only immunostimulatory when administered in combination with a lipid particle, and is not immunostimulatory when administered in its “free form.” Such an oligonucleotide is considered to be immunostimulatory.

Immunostimulatory isolated oligonucleotides are considered to be non-sequence specific when it is not required that they specifically bind to and reduce the expression of a target polynucleotide in order to provoke an immune response. Thus, certain immunostimulatory isolated oligonucleotide may comprise a sequence corresponding to a region of a naturally occurring gene or mRNA, but they may still be considered non-sequence specific immunostimulatory nucleic acids.

In some embodiments, the immunostimulatory isolated oligonucleotide comprises at least one CpG dinucleotide. The oligonucleotide or CpG dinucleotide may be unmethylated or methylated. In another embodiment, the immunostimulatory isolated oligonucleotide comprises at least one CpG dinucleotide having a methylated cytosine. In some embodiments, the nucleic acid comprises a single CpG dinucleotide, wherein the cytosine in said CpG dinucleotide is methylated. In an alternative embodiment, the isolated oligonucleotide comprises at least two CpG dinucleotides, wherein at least one cytosine in the CpG dinucleotides is methylated. In a further embodiment, each cytosine in the CpG dinucleotides present in the sequence is methylated. In another embodiment, the isolated oligonucleotide comprises a plurality of CpG dinucleotides, wherein at least one of said CpG dinucleotides comprises a methylated cytosine.

Attachments Between Linker Unit, isolated oligonucleotide, and Ligand

In some embodiments, the attachment between the Linker Unit and the Nucleic Acid Agent is a bond.

In some embodiments, the attachment between the Linker Unit and the Nucleic Acid Agent is a moiety (e.g., a moiety comprising a cleavable group).

In some embodiments, the attachment between the Linker Unit and the ligand is a bond.

In some embodiments, the attachment between the Linker Unit and the ligand is a moiety (e.g., a moiety comprising a cleavable group).

In some embodiments, the attachment between the Linker Unit and the ligand comprises —C(═O)— connected to the Linker Unit.

The group can be cleavable or non-cleavable. Suitable groups include, for example, —NR—, —C(═O)—, —C(═O)NH—, —S(═O)—, —S(═O)₂—, —S(═O)₂NH— or a chain of atoms, such as, but not limited to, alkylene, alkenylene, alkynylene, arylalkylene, arylalkenylene, arylalkynylene, heteroarylalkylene, heteroarylalkenylene, heteroarylalkynylene, heterocyclylalkylene, heterocyclylalkenylene, heterocyclylalkynylene, arylene, heteroarylene, heterocyclylene, cycloalkylene, cycloalkenylene, alkylarylalkylene, alkylarylalkenylene, alkylarylalkynylene, alkenylarylalkylene, alkenylarylalkenylene, alkenylarylalkynylene, alkynylarylalkylene, alkynylarylalkenylene, alkynylarylalkynylene, alkylheteroarylalkylene, alkylheteroarylalkenylene, alkylheteroarylalkynylene, alkenylheteroarylalkylene, alkenylheteroarylalkenylene, alkenylheteroarylalkynylene, alkynylheteroarylalkylene, alkynylheteroarylalkenylene, alkynylheteroarylalkynylene, alkylheterocyclylalkylene, alkylheterocyclylalkenylene, alkylhererocyclylalkynylene, alkenylheterocyclylalkylene, alkenylheterocyclylalkenylene, alkenylheterocyclylalkynylene, alkynylheterocyclylalkylene, alkynylheterocyclylalkenylene, alkynylheterocyclylalkynylene, alkylarylene, alkenylarylene, alkynylarylene, alkylheteroarylene, alkenylheteroarylene, alkynylhereroarylene, each of which may be substituted or unsubstituted, and which one or more methylenes can be interrupted or terminated by —O—, —S—, —S(═O)—, —S(═O)₂—, —NR—, —C(═O)—, substituted or unsubstituted aryl, substituted or unsubstituted heteroaryl, or substituted or unsubstituted heterocyclic, where R is hydrogen, acyl, aliphatic or substituted aliphatic.

A cleavable group is one which is sufficiently stable outside the cell, but which upon entry into a target cell is cleaved to release the two parts the group is holding together. In a preferred embodiment, the cleavable group is cleaved at least 10 times or more, preferably at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable group by reduction; esterases; endosomes or agents that can create an acidic environment, e.g., those that result in a pH of five or lower; enzymes that can hydrolyze or degrade an acid cleavable group by acting as a general acid, peptidases (which can be substrate specific), and phosphatases.

A cleavable group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable group that is cleaved at a preferred pH, thereby releasing the cationic lipid from the ligand inside the cell, or into the desired compartment of the cell.

A conjugate can include a cleavable group that is cleavable by a particular enzyme. The type of cleavable group incorporated into a conjugate can depend on the cell to be targeted. For example, liver targeting ligands can be attached to the cationic lipids through a chemical moiety that includes an ester group. Liver cells are rich in esterases, and therefore the group will be cleaved more efficiently in liver cells than in cell types that are not esterase-rich. Other cell-types rich in esterases include cells of the lung, renal cortex, and testis.

Coupling groups that contain peptide bonds can be used when targeting cell types rich in peptidases, such as liver cells and synoviocytes.

In general, the suitability of a candidate cleavable group can be evaluated by testing the ability of a degradative agent (or condition) to cleave the candidate group. It will also be desirable to also test the candidate cleavable group for the ability to resist cleavage in the blood or when in contact with other non-target tissue. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

Redox Cleavable Groups. One class of cleavable groups are redox cleavable groups that are cleaved upon reduction or oxidation. An example of reductively cleavable group is a disulphide linking group (—S—S—). To determine if a candidate cleavable group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

Phosphate-Based Cleavable Groups. Phosphate-based cleavable groups are cleaved by agents that degrade or hydrolyze the phosphate group. An example of an agent that cleaves phosphate groups in cells are enzymes such as phosphatases in cells. In some embodiments, the phosphate-based linking group is —O—P(═O)(ORk)-O—, —O—P(═S)(ORk)-O—, —O—P(═S)(SRk)-O—, —S—P(═O)(ORk)-O—, —O—P(═O)(ORk)-S—, —S—P(═O)(ORk)-S—, —O—P(═S)(ORk)-S—, —S—P(═S)(ORk)-O—, —O—P(═O)(Rk)-O—, —O—P(═S)(Rk)-O—, —S—P(═O)(Rk)-O—, —S—P(═S)(Rk)-O—, —S—P(═O)(Rk)-S—, or —O—P(═S)(Rk)-S—. In some embodiments, the phosphate-based linking group is —O—P(═O)(OH)—O—, —O—P(═S)(OH)—O—, —O—P(═S)(SH)—O—, —S—P(—O)(OH)—O—, —O—P(═O)(OH)—S—, —S—P(—O)(OH)—S—, —O—P(═S)(OH)—S—, —S—P(═S)(OH)—O—, —O—P(═O)(H)—O—, —O—P(═S)(H)—O—, —S—P(═O)(H)—O—, —S—P(═S)(H)—O—, —S—P(═O)(H)—S—, or —O—P(═S)(H)—S—. In some embodiments, the phosphate-based linking group is —O—P(—O)(OH)—O—.

Acid Cleavable Groups. Acid cleavable groups are linking groups that are cleaved under acidic conditions. In preferred embodiments acid cleavable groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

Ester-Based Cleavable Groups. Ester-based cleavable groups are cleaved by enzymes such as esterases and amidases in cells. Examples of ester-based cleavable groups include but are not limited to esters of alkylene, alkenylene and alkynylene groups. Ester cleavable linking groups have the general formula —C(O)O—, or —OC(O)—. These candidates can be evaluated using methods analogous to those described above.

Peptide-Based Cleavable Groups. Peptide-based cleavable groups are cleaved by enzymes such as peptidases and proteases in cells. Peptide-based cleavable groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above. As used herein, “carbohydrate” refers to a compound which is either a carbohydrate per se made up of one or more monosaccharide units having at least 6 carbon atoms (which may be linear, branched or cyclic) with an oxygen, nitrogen or sulfur atom bonded to each carbon atom; or a compound having as a part thereof a carbohydrate moiety made up of one or more monosaccharide units each having at least six carbon atoms (which may be linear, branched or cyclic), with an oxygen, nitrogen or sulfur atom bonded to each carbon atom. Representative carbohydrates include the sugars (mono-, di-, tri- and oligosaccharides containing from about 4-9 monosaccharide units), and polysaccharides such as starches, glycogen, cellulose and polysaccharide gums. Specific monosaccharides include C5 and above (preferably C5-C8) sugars; di- and trisaccharides include sugars having two or three monosaccharide units (preferably C5-C8).

In some embodiments, the antisense strand of the isolated oligonucleotide of the present disclosure is complementary to an mRNA, wherein the sequence-specific hybridization to the antisense strand to the mRNA results in degradation of the mRNA.

### Methods of Preparing Isolated Oligonucleotides of the Present Disclosure

In some aspects, the present disclosure provides a pharmaceutical composition comprising an isolated oligonucleotide or a compound, a scaffold, or conjugate comprising an isolated oligonucleotide, described herein.

In some aspects, the present disclosure provides a method of modulating the expression of a target gene in a subject, comprising administering to the subject an isolated oligonucleotide or a compound, a scaffold, or conjugate comprising an isolated oligonucleotide, described herein described herein.

In some aspects, the present disclosure provides a method of delivering a Nucleic Acid Agent to a subject, comprising administering to the subject an isolated oligonucleotide or a compound, a scaffold, or conjugate comprising an isolated oligonucleotide, described herein described herein.

In some aspects, the present disclosure provides a method of treating or preventing a disease in a subject in need thereof, comprising administering to the subject a therapeutically effective amount of an isolated oligonucleotide or a compound, a scaffold, or conjugate comprising an isolated oligonucleotide, described herein described herein.

In some aspects, the present disclosure provides a use of an isolated oligonucleotide or a compound, a scaffold, or conjugate comprising an isolated oligonucleotide, described herein described herein in the manufacture of a medicament for modulating the expression of a target gene in a subject.

In some aspects, the present disclosure provides a use of an isolated oligonucleotide or a compound, a scaffold, or conjugate comprising an isolated oligonucleotide, described herein described herein in the manufacture of a medicament for delivering a Nucleic Acid Agent to a subject.

In some aspects, the present disclosure provides a use of an isolated oligonucleotide or a compound, a scaffold, or conjugate comprising an isolated oligonucleotide, described herein described herein in the manufacture of a medicament for treating or preventing a disease in a subject in need thereof.

In some embodiments, the isolated oligonucleotide of the present disclosure comprises one or more one or more phosphate groups or one or more analogs of a phosphate group.

In some embodiments, the isolated oligonucleotide is an siRNA (e.g., a single strand siRNA (e.g., a hairpin single strand siRNA) or a double strand siRNA), microRNA, antimicroRNA, microRNA mimics, antimiR, antagomir, dsRNA, aptamer, immune stimulatory oligonucleotide, decoy oligonucleotide, splice altering oligonucleotide, triplex forming oligonucleotide, G-quadruplex, or antisense oligonucleotide.

In some embodiments, the isolated oligonucleotide a double stranded RNA (dsRNA), wherein the double stranded RNA.

It is understood that sense strand of the isolated oligonucleotide of the present disclosure is also known as passenger strand, and the terms “sense strand” and “passenger strand” are used interchangeably herein.

It is understood that antisense strand of the isolated oligonucleotide of the present disclosure is also known as guide strand, and the terms “antisense strand” and “guide strand” are used interchangeably herein.

In some embodiments, the isolated oligonucleotide is an iRNA.

The term “iRNA” refers to an RNA agent which can down regulate the expression of a target gene (e.g., an siRNA), e.g., an endogenous or pathogen target RNA. While not wishing to be bound by theory, an iRNA may act by one or more of a number of mechanisms, including post-transcriptional cleavage of a target mRNA (referred to in the art as RNAi), or pre-transcriptional or pre-translational mechanisms. An iRNA can include a single strand or can include more than one strands, e.g., it can be a double stranded iRNA. If the iRNA is a single strand it can include a 5′ modification which includes one or more phosphate groups or one or more analogs of a phosphate group. In some embodiments, the iRNA is double stranded. In some embodiments, one or both strands of the double stranded iRNA can be modified, e.g., 5′ modification.

The iRNA typically includes a region of sufficient homology to the target gene, and is of sufficient length in terms of nucleotides, such that the iRNA, or a fragment thereof, can mediate down regulation of the target gene. The iRNA is or includes a region which is at least partially, and in some embodiments fully, complementary to the target RNA. It is not necessary that there be perfect complementarity between the iRNA and the target, but the correspondence may be sufficient to enable the iRNA, or a cleavage product thereof, to direct sequence specific silencing, e.g., by RNAi cleavage of the target RNA, e.g., mRNA.

The nucleotides of the isolated oligonucleotide may be further modified. The single stranded or double stranded regions of an isolated oligonucleotide may be further modified or further include nucleotide surrogates, e.g., the unpaired region or regions of a hairpin structure, e.g., a region which links two complementary regions, can have modifications or nucleotide surrogates. Modification to stabilize one or more 3′- or 5′-terminus of an isolated oligonucleotide, e.g., against exonucleases. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis. Further modifications can also include, e.g., the use of modifications at the 2′ OH group of the ribose sugar, e.g., the use of deoxyribonucleotides, e.g., deoxythymidine, instead of ribonucleotides, and modifications in the phosphate group, e.g., phosphothioate modifications. In some embodiments, the different strands will include different further modifications.

In some embodiments, the length for the duplexed regions between the strands of the isolated oligonucleotide are between 6 and 30 nucleotides in length. In some embodiments, the duplexed regions are between 15 and 30, most preferably 18, 19, 20, 21, 22, and 23 nucleotides in length. In some embodiments, the duplexed regions are between 6 and 20 nucleotides, most preferably 6, 7, 8, 9, 10, 11 and 12 nucleotides in length.

The isolated oligonucleotide may be that described in U.S. Patent Publication Nos. 2009/0239814, 2012/0136042, 2013/0158824, or 2009/0247608, each of which is hereby incorporated by reference.

In some embodiments, the isolated oligonucleotide is an siRNA.

In some embodiments, the isolated oligonucleotide is a double strand siRNA, for example, double strand siRNA described herein.

A “double stranded siRNA” as used herein, is an siRNA which includes more than one, and in some cases two, strands in which interchain hybridization can form a region of duplex structure. In some embodiments, the sense strand of a double stranded siRNA may be equal to or at least 13, 14, 15, 16 17, 18, 19, 20, 21, 22, 23, 24, 25, 29, 30, 31, 32, 33, 34, 35 or 36 nucleotides in length. In some embodiments, the antisense strand of a double stranded siRNA may be equal to or at least, 18, 19, 20, 21, 22, 23, 24, 25, 29, 30 or 31 nucleotides in length.

In some embodiments, the isolated oligonucleotide of the present disclosure can be converted to or dissociated to form a single strand siRNA.

A “single strand siRNA” as used herein, is an siRNA which is made up of a single strand, which includes a duplexed region, formed by intra-strand pairing, e.g., it may be, or include, a hairpin or pan-handle structure. Single strand siRNAs may be antisense with regard to the target molecule. A single strand siRNA may be sufficiently long that it can enter the RISC and participate in RISC mediated cleavage of a target mRNA. A single strand siRNA is at least 13, 14, 15, 16 17, 18, 19, 20, 21, 22, 23, 24, 25, 29, 30, 31, 32, 33, 34, 35 or 36 nucleotides in length. A single strand siRNA is at least 18, 19, 20, 21, 22, 23, 24, 25, 29, 30 or 31 nucleotides in length.

In some embodiments, the isolated oligonucleotide is an Hairpin siRNA.

Hairpin siRNAs may have a duplex region equal to or at least 17, 18, 19, 20, 21, 22, 23, 24, or 25 nucleotide pairs. In some embodiments, ranges for the duplex region are 15-30, 17 to 23, 19 to 23, 18-21, and 19 to 21 nucleotides pairs in length. The hairpin may have a single strand overhang or terminal unpaired region. In some embodiments, the overhangs are at least two in length. In some embodiments, the overhang is at the antisense side of the hairpin.

In some embodiments, the siRNA is sufficiently large that it can be cleaved by an endogenous molecule, e.g., by Dicer, to produce smaller siRNAs, e.g., siRNAs agents

The sense and antisense strands may be chosen such that the double-stranded siRNA includes a single strand or unpaired region at one end of the molecule. Thus, a double-stranded siRNA may contain sense and antisense strands, paired to contain a 3′ overhang of 2 or more nucleotides. In some embodiments, the overhang is 2 nucleotides.

The siRNAs described herein, including double-stranded siRNAs and a single-stranded siRNAs generated from the double-stranded siRNA, can mediate silencing of a target RNA, e.g., mRNA, e.g., a transcript of a gene that encodes a protein. For convenience, such mRNA is also referred to herein as mRNA to be silenced. Such a gene is also referred to as a target gene. In general, the RNA to be silenced is an endogenous gene or a pathogen gene. In addition, RNAs other than mRNA, e.g., tRNAs, and viral RNAs, can also be targeted.

As used herein, the phrases “mediates RNAi” or “mRNA silencing” or “targeting mRNA” “inhibiting mRNA” or “degrading mRNA” refer to the ability to silence, in a sequence specific manner, a target RNA. While not wishing to be bound by theory, it is believed that silencing uses the RNAi machinery or process and a guide RNA, e.g., an ssRNA of 21 to 23 nucleotides.

In some embodiments, the isolated oligonucleotide is an siRNA that is “sufficiently complementary” to a target RNA, e.g., a target mRNA, such that the siRNA silences production of protein encoded by the target mRNA. In another embodiment, the siRNA is “exactly complementary” to a target RNA, e.g., the target RNA and the siRNA anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in some embodiments, the siRNA specifically discriminates a single-nucleotide difference. In this case, the siRNA only mediates RNAi if exact complementary is found in the region (e.g., within 7 nucleotides of) the single-nucleotide difference.

In some embodiments, the isolated oligonucleotide is an antisense oligonucleotide directed to a target polynucleotide. The term “antisense oligonucleotide” or simply “antisense” is meant to include oligonucleotides that are complementary to a targeted polynucleotide sequence. Antisense oligonucleotides are single strands of DNA or RNA that are complementary to a chosen sequence, e.g., a target gene mRNA. Antisense oligonucleotides are thought to inhibit gene expression by binding to a complementary mRNA. Binding to the target mRNA can lead to inhibition of gene expression either by preventing translation of complementary mRNA strands by binding to it, or by leading to degradation of the target mRNA. In some embodiments, antisense oligonucleotides contain from about 13 to about 36 nucleotides, more preferably about 20 to about 25 nucleotides. The term also encompasses antisense oligonucleotides that may not be exactly complementary to the desired target gene. Thus, instances where non-target specific-activities are found with antisense, or where an antisense sequence containing one or more mismatches with the target sequence is the most preferred for a particular use, are contemplated.

Antisense oligonucleotides have been demonstrated to be effective and targeted inhibitors of protein synthesis, and, consequently, can be used to specifically inhibit protein synthesis by a targeted gene. The efficacy of antisense oligonucleotides for inhibiting protein synthesis is well established. For example, the synthesis of polygalacturonase and the muscarine type 2 acetylcholine receptor are inhibited by antisense oligonucleotides directed to their respective mRNA sequences (U.S. Pat. Nos. 5,739,119 and 5,759,829 each of which is incorporated by reference). Further, examples of antisense inhibition have been demonstrated with the nuclear protein cyclin, the multiple drug resistance gene (MDG1), ICAM-1, E-selectin, STK-1, striatal GABAA receptor and human EGF (Jaskulski et al., Science. 1988 Jun. 10; 240(4858):1544-6; Vasanthakumar and Ahmed, Cancer Commun. 1989; 1(4):225-32; Peris et al., Brain Res Mol Brain Res. 1998 Jun. 15; 57(2):310-20; U.S. Pat. Nos. 5,801,154; 5,789,573; 5,718,709 and 5,610,288, each of which is incorporated by reference). Furthermore, antisense constructs have also been described that inhibit and can be used to treat a variety of abnormal cellular proliferations, e.g., cancer (U.S. Pat. Nos. 5,747,470; 5,591,317 and 5,783,683, each of which is incorporated by reference).

Methods of producing antisense oligonucleotides are known in the art and can be readily adapted to produce an antisense oligonucleotide that targets any polynucleotide sequence. Selection of antisense oligonucleotide sequences specific for a given target sequence is based upon analysis of the chosen target sequence and determination of secondary structure, Tm, binding energy, and relative stability. Antisense oligonucleotides may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences that are substantially complementary to 5′ regions of the mRNA. These secondary structure analyses and target site selection considerations can be performed, for example, using v.4 of the OLIGO primer analysis software (Molecular Biology Insights) and/or the BLASTN 2.0.5 algorithm software (Altschul et al., Nucleic Acids Res. 1997, 25(17):3389-402).

In some embodiments, the isolated oligonucleotide can be an antagomir. Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate backbone and, for example, a cholesterol-moiety at 3′-end. Antagomirs may be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al, Nature, 2005, 438: 685-689, which is expressly incorporated by reference herein in its entirety. Antagomir RNAs may be synthesized using standard solid phase oligonucleotide synthesis protocols. See U.S. Patent Application Publication Nos. 2007/0123482 and 2007/0213292 (each of which is incorporated herein by reference).

An antagomir can include ligand-conjugated monomer subunits and monomers for oligonucleotide synthesis. Exemplary monomers are described in U.S. Patent Application Publication No. 2005/0107325, which is incorporated by reference in its entirety. An antagomir can have a ZXY structure, such as is described in WO 2004/080406, which is incorporated by reference in its entirety. An antagomir can be complexed with an amphipathic moiety. Exemplary amphipathic moieties for use with oligonucleotide agents are described in WO 2004/080406, which is incorporated by reference in its entirety.

In some embodiments, the isolated oligonucleotides of the present disclosure linked to a lipid, are associated with ribozymes. Ribozymes are RNA molecules complexes having specific catalytic domains that possess endonuclease activity (Kim and Cech, Proc Natl Acad Sci USA. 1987 December; 84(24):8788-92; Forster and Symons, Cell. 1987 Apr. 24; 49(2):211-20). For example, a large number of ribozymes accelerate phosphoester transfer reactions with a high degree of specificity, often cleaving only one of several phosphoesters in an oligonucleotide substrate (Cech et al., Cell. 1981 December; 27(3 Pt 2):487-96; Michel and Westhof, J Mol Biol. 1990 Dec. 5; 216(3):585-610; Reinhold-Hurek and Shub, Nature. 1992 May 14; 357(6374):173-6). This specificity has been attributed to the requirement that the substrate bind via specific base-pairing interactions to the internal guide sequence (“IGS”) of the ribozyme prior to chemical reaction.

At least six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nucleic acid molecule may be formed in a hammerhead, hairpin, a hepatitis 6 virus, group I intron or RNaseP RNA (in association with an RNA guide sequence) or Neurospora VS RNA motif, for example. Specific examples of hammerhead motifs are described by Rossi et al. Nucleic Acids Res. 1992 Sep. 11; 20(17):4559-65. Examples of hairpin motifs are described by Hampel et al. (Eur. Pat. Appl. Publ. No. EP 0360257), Hampel and Tritz, Biochemistry 1989 Jun. 13; 28(12):4929-33; Hampel et al., Nucleic Acids Res. 1990 Jan. 25; 18(2):299-304 and U.S. Pat. No. 5,631,359. An example of the hepatitis 6 virus motif is described by Perrotta and Been, Biochemistry. 1992 Dec. 1; 31(47):11843-52; an example of the RNaseP motif is described by Guerrier-Takada et al., Cell. 1983 December; 35(3 Pt 2):849-57; Neurospora VS RNA ribozyme motif is described by Collins (Saville and Collins, Cell. 1990 May 18; 61(4):685-96; Saville and Collins, Proc Natl Acad Sci USA. 1991 Oct. 1; 88(19):8826-30; Collins and Olive, Biochemistry. 1993 Mar. 23; 32(11):2795-9); and an example of the Group I intron is described in U.S. Pat. No. 4,987,071. Important characteristics of enzymatic nucleic acid molecules used are that they have a specific substrate binding site which is complementary to one or more of the target gene DNA or RNA regions, and that they have nucleotide sequences within or surrounding that substrate binding site which impart an RNA cleaving activity to the molecule. Thus, the ribozyme constructs need not be limited to specific motifs mentioned herein.

Methods of producing a ribozyme targeted to any polynucleotide sequence are known in the art. Ribozymes may be designed as described in Int. Pat. Appl. Publ. Nos. WO 93/23569 and WO 94/02595, each specifically incorporated herein by reference, and synthesized to be tested in vitro and in vivo, as described therein.

Ribozyme activity can be optimized by altering the length of the ribozyme binding arms or chemically synthesizing ribozymes with modifications that prevent their degradation by serum ribonucleases (see e.g., Int. Pat. Appl. Publ. Nos. WO 92/07065, WO 93/15187, and WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

### Methods of Synthesis

In some aspects, the present disclosure provides a method of preparing an isolated oligonucleotide or a compound, a conjugate or a scaffold comprising an isolated oligonucleotide of the present disclosure.

In some aspects, the present disclosure provides an intermediate as described herein, being suitable for use in a method for preparing an isolated oligonucleotide or a compound, a conjugate or a scaffold comprising an isolated oligonucleotide of the present disclosure.

The isolated oligonucleotide or a compound, a conjugate or a scaffold comprising an isolated oligonucleotide of the present disclosure can be prepared by any suitable technique known in the art. Particular processes for the preparation of an isolated oligonucleotide or a compound, a conjugate or a scaffold comprising an isolated oligonucleotide of the present disclosure are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art. It is understood by one skilled in the art of organic synthesis that the functionality present on various portions of the molecule must be compatible with the reagents and reaction conditions utilized.

It will be appreciated that during the synthesis of the isolated oligonucleotide or a compound, a conjugate or a scaffold comprising an isolated oligonucleotide of the present disclosure, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. A suitable protecting group for an hydroxy or alkylhydroxy group can be, e.g., Acetyl (Ac), Benzoyl (Bz), Benzyl (Bn), β-Methoxyethoxymethyl ether (MEM), Dimethoxytrityl (DMT), Methoxymethyl ether (MOM), Methoxytrityl (MMT), p-Methoxybenzyl ether (PMB), p-Methoxyphenyl ether (PMP), Pivaloyl (Piv), Tetrahydropyranyl (THP), Tetrahydrofuran (THF), Trityl (triphenylmethyl, Tr), Silyl ether (e.g., trimethylsilyl (TMS), tert-butyldimethylsilyl (TBDMS), tri-iso-propylsilyloxymethyl (TOM), and triisopropylsilyl (TIPS) ethers), a Methyl ether, or an Ethoxyethyl ether (EE). A suitable protecting group for an 1,2-diol can be, e.g., acetal. A suitable protecting group for an 1,3-diol can be, e.g., tetraisopropyldisiloxanylidene (TIPDS).

The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively, an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, an isolated oligonucleotide or a compound, a conjugate or a scaffold comprising an isolated oligonucleotide of the present disclosure, are readily accessible by various synthetic routes, some of which are exemplified in the accompanying examples. The skilled person will easily recognize which kind of reagents and reactions conditions are to be used and how they are to be applied and adapted in any particular instance—wherever necessary or useful—in order to obtain the compounds of the present disclosure. Furthermore, some of the an isolated oligonucleotide or a compound, a conjugate or a scaffold comprising an isolated oligonucleotide of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

### Biological Assays

An isolated oligonucleotide or a compound, a conjugate or a scaffold comprising an isolated oligonucleotide of the present disclosure, designed, selected, prepared and/or optimized by methods described above, once produced, can be characterized using a variety of assays known to those skilled in the art to determine whether the compounds, scaffolds, or conjugates have biological activity. For example, the isolated oligonucleotide or a compound, a conjugate or a scaffold comprising the isolated oligonucleotide of the present disclosure, can be characterized by conventional assays, including but not limited to those assays described below, to determine whether they have a desired activity, e.g., target binding activity and/or specificity and/or stability.

Furthermore, high-throughput screening can be used to speed up analysis using such assays. As a result, it may be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds, scaffolds, or conjugates of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments, the biological assays are described in the Examples herein.

### Pharmaceutical Compositions

In some aspects, the present disclosure provides a pharmaceutical composition comprising an isolated oligonucleotide or a compound, a conjugate or a scaffold comprising an isolated oligonucleotide of the present disclosure of the present disclosure as an active ingredient.

As used herein, the term “composition” is intended to encompass a product comprising the specified ingredients in the specified amounts, as well as any product which results, directly or indirectly, from combination of the specified ingredients in the specified amounts.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and F-aminocaproic acid, and mixtures thereof.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

An effective amount of a compound of the present disclosure for use in therapy is an amount sufficient to treat or prevent an inflammasome related condition referred to herein, slow its progression and/or reduce the symptoms associated with the condition.

An effective amount of a compound of the present disclosure for use in therapy is an amount sufficient to treat an inflammasome related condition referred to herein, slow its progression and/or reduce the symptoms associated with the condition.

The size of the dose for therapeutic or prophylactic purposes of a compound of Formula (I) or (II) will naturally vary according to the nature and severity of the conditions, the age and sex of the animal or patient and the route of administration, according to well-known principles of medicine. It is to be understood that the present disclosure also provides pharmaceutical compositions comprising any compound, scaffold, or conjugate described herein in combination with at least one pharmaceutically acceptable excipient or carrier.

As used herein, the term “pharmaceutical composition” is a formulation containing the compounds, scaffolds, or conjugates of the present disclosure in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In some embodiments, the active compound is mixed under sterile conditions with a pharmaceutically acceptable carrier, and with any preservatives, buffers, or propellants that are required.

As used herein, the term “pharmaceutically acceptable” refers to those compounds, scaffolds, conjugates, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

It is to be understood that compounds, scaffolds, and conjugates of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5th edition, John Wiley & Sons: New York, 2001; Greene, T. W., Wuts, P. G. M., Protective Groups in Organic Synthesis, 3rd edition, John Wiley & Sons: New York, 1999; R. Larock, Comprehensive Organic Transformations, VCH Publishers (1989); L. Fieser and M. Fieser, Fieser and Fieser's Reagents forganic Synthesis, John Wiley and Sons (1994); and L. Paquette, ed., Encyclopedia of Reagents forganic Synthesis, John Wiley and Sons (1995), incorporated by reference herein, are useful and recognised reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3rd edition, John Wiley & Sons: New York, 1999.

Techniques for formulation and administration of the disclosed compounds of the disclosure can be found in Remington: the Science and Practice of Pharmacy, 19th edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous organic solutions. The compounds will be present in such pharmaceutical compositions in amounts sufficient to provide the desired dosage amount in the range described herein.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL□ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze drying that yields a powder of the active ingredient plus any additional desired ingredient from a previously sterile filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, orange flavoring.

For administration by inhalation, the compounds are delivered in the form of an aerosol spray from pressured container or dispenser, which contains a suitable propellant, e.g., a gas such as carbon dioxide, or a nebuliser.

For intranasal administration, the compounds are delivered in solution or solid formulation. In some embodiments, the compounds are delivered in solution as a mist, a drip, or a swab. In some embodiments, the compounds are delivered as a powder. In some embodiments, the compound is included in a kit which further includes an intranasal applicator.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure is dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds, scaffolds, or conjugates of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral organic acid salts of basic residues such as amines, alkali organic salts of acidic residues such as carboxylic acids, and the like. The pharmaceutically acceptable salts include the conventional non-toxic salts or the quaternary ammonium salts of the parent compound formed, for example, from non-toxic inorganic organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In some embodiments, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

### Methods of Use

In some aspects, the present disclosure provides a method of modulating (e.g., reducing or eliminating) the expression of a target gene in a subject, comprising administering to the subject an isolated oligonucleotide or a compound, a conjugate or a scaffold comprising an isolated oligonucleotide of the present disclosure.

In some aspects, the present disclosure provides a method of modulating (e.g., reducing or eliminating) the expression of a target gene in a cell or tissue of a subject, comprising administering to the subject an isolated oligonucleotide or a compound, a conjugate or a scaffold comprising an isolated oligonucleotide of the present disclosure.

In some aspects, the present disclosure provides a method of delivering an isolated oligonucleotide or a compound, a conjugate or a scaffold comprising an isolated oligonucleotide or a compound, a conjugate or a scaffold comprising an isolated oligonucleotide of the present disclosure to a subject, comprising administering to the subject the isolated oligonucleotide or the compound, a conjugate or a scaffold comprising an isolated oligonucleotide of the present disclosure.

In some aspects, the present disclosure provides a method of treating or preventing a disease in a subject in need thereof, comprising administering to the subject a therapeutically effective amount of an isolated oligonucleotide or a compound, a conjugate or a scaffold comprising an isolated oligonucleotide of the present disclosure.

In some aspects, the present disclosure provides an isolated oligonucleotide or a compound, a conjugate or a scaffold comprising an isolated oligonucleotide of the present disclosure for modulating (e.g., reducing or eliminating) the expression of a target gene in a subject.

In some aspects, the present disclosure provides an isolated oligonucleotide or a compound, a conjugate or a scaffold comprising an isolated oligonucleotide of the present disclosure for modulating (e.g., reducing or eliminating) the expression of a target gene in a cell or tissue of a subject.

In some aspects, the present disclosure provides a compound, a conjugate or a scaffold comprising an isolated oligonucleotide of the present disclosure for delivering an isolated oligonucleotide of the present disclosure.

In some aspects, the present disclosure provides an isolated oligonucleotide or a compound, a conjugate or a scaffold comprising an isolated oligonucleotide of the present disclosure, for treating or preventing a disease in a subject in need thereof.

In some aspects, the present disclosure provides use of an isolated oligonucleotide or a compound, a conjugate or a scaffold comprising an isolated oligonucleotide of the present disclosure, in the manufacture of a medicament for modulating (e.g., reducing or eliminating) the expression of a target gene in a subject.

In some aspects, the present disclosure provides use of an isolated oligonucleotide or a compound, a conjugate or a scaffold comprising an isolated oligonucleotide of the present disclosure, in the manufacture of a medicament for modulating (e.g., reducing or eliminating) the expression of a target gene in a cell or tissue of a subject.

In some aspects, the present disclosure provides use of a compound, a conjugate or a scaffold comprising an isolated oligonucleotide of the present disclosure, in the manufacture of a medicament for delivering the isolated oligonucleotide of the present disclosure to a subject.

In some aspects, the present disclosure provides use of an isolated oligonucleotide of the present disclosure or a compound, a conjugate or a scaffold comprising an isolated oligonucleotide of the present disclosure, in the manufacture of a medicament for treating or preventing a disease in a subject in need thereof.

In some embodiments, the subject is a cell. In some embodiments, the subject is a tissue. In some embodiments, the subject is a human.

In some embodiments, an modified siRNA molecule with the chemically modified sense and antisense strands of the present disclosure can be directed against any target gene, for example, PCSK9, ANGPTL-3, AGT, HSD17b3, C3, CfB, APOC3, C5, SOD1, GPAM, LPA, F11, Factor VII, Eg5, TPX2, apoB, SAA, TTR, HBV, HCV, RSV, PDGF beta gene, Erb-B gene, Src gene, CRK gene, GRB2 gene, RAS gene, MEKK gene, JNK gene, RAF gene, Erkl/2 gene, PCNA(p21) gene, MYB gene, JUN gene, FOS gene, BCL-2 gene, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, p73 gene, p21(WAF1/CIP1) gene, p27(KIP1) gene, PPM1D gene, RAS gene, caveolin I gene, MIB I gene, MTAI gene, M68 gene, mutations in tumor suppressor genes, p53 tumor suppressor gene, LDHA, or any combination thereof.

In some embodiments, the disease characterized by unwanted or aberrant expression of the target gene. In some embodiments, the administration results in reduced or eliminated expression of the target gene in the subject.

In some embodiments, the disease is a viral infection, e.g., an HCV, HBV, HPV, HSV or HIV infection.

In some embodiments, the disease is cancer. In some embodiments, the cancer is biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, wilms tumor, leukemia, acute lymphocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T-cell lymphomas, non-Hodgkin lymphoma, precursor T-lymphoblastic lymphoma/leukemia, peripheral T-cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

In some embodiments, the cancer is liver cancer, liver carcinoma, hepatoma, hepatocellular carcinoma, cholangiocarcinoma, orhepatoblastoma.

In some embodiments, the disease is a proliferative, inflammatory, autoimmune, neurologic, ocular, respiratory, metabolic, dermatological, auditory, liver, kidney, or infectious disease. In some embodiments, the disease is a disease of the liver. In some embodiments, the disease of the liver is cirrhosis, steatosis or a combination thereof.

In some embodiments, the disease is a metabolic disorder. In some embodiments, the metabolic disorder is hypercholesterolemia, hypobetalipoproteinemia, coronary heart disease, peripheral arterial disease, stroke, type 2 diabetes, obesity, or high blood pressure or a combination thereof.

It is to be understood that, unless otherwise stated, any description of a method of treatment or prevention includes use of the compounds, scaffolds, and conjugates to provide such treatment or prevention as is described herein. It is to be further understood, unless otherwise stated, any description of a method of treatment or prevention includes use of the compounds, scaffolds, and conjugates to prepare a medicament to treat or prevent such condition. The treatment or prevention includes treatment or prevention of human or non-human animals including rodents and other disease models.

It is to be understood that, unless otherwise stated, any description of a method of treatment includes use of the compounds, scaffolds, and conjugates to provide such treatment as is described herein. It is to be further understood, unless otherwise stated, any description of a method of treatment includes use of the compounds, scaffolds, and conjugates to prepare a medicament to treat such condition. The treatment includes treatment of human or non-human animals including rodents and other disease models.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In some embodiments, the mammal is a human. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that compounds, scaffolds, and conjugates of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

As used herein, the term “therapeutically effective amount”, refers to an amount of a pharmaceutical agent to treat or ameliorate an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1: Optimization of Chemical Modification Pattern to Improve Stability and Potency of siRNAs Targeting Gene 1

Described herein is a study showing further improvement in the stability and tolerability of siRNAs by reducing the 2′-fluoro nucleotide content and concomitantly increasing 2′-O-methyl content. The 2′-fluoro and 2′-O-methyl content across both the sense and antisense strands of siRNAs was systemically examined and advanced modification designs with improved in vitro activity and metabolic stability were identified, which can be translated to enhanced potency, duration, and tolerability in vivo. In the study described herein, the positional effects of 2′-O-methyl and 2′-fluoro modification on the sense and antisense strands systematically examined, and several advanced designs with significantly improved in vitro activity were identified for both 21/23mer and 20/22mer platforms.

The study described herein used a publicly reported prototype compound (21/23mer), which is a clinically approved drug. Based on the chemical modification of the prototype compound, the positional effects of the 2′-O-methyl and 2′-fluoro modification on both sense and antisense strands were systemically examined for both 21/23mer and 20/22mer platforms. Described herein are several advanced designs with up to 60-fold improvement of in vitro activity compared with that of the prototype compound (IC₅₀s of 4 pm vs 245 pm).

### Materials and Methods

Compounds (e.g., Nucleic Acid Agents, Nucleic Acid Agents containing Nucleotide-Based Enhancement Agents, and Conjugates) were prepared by solid-phase synthesis according to standard synthesis protocols.

Briefly, oligonucleotide synthesis was conducted on a solid support to incorporate each nucleoside phosphoramidites from 3′-end to 5′-end to prepare oligo single strands. ETT or BTT was used as an activator for the coupling reaction. Iodine in water/pyridine/THF was used to oxidize phosphite-triester (P(III)) to afford phosphate backbones and DDTT was used for the preparation of phosphorothioate linkages. Aqueous ammonium was used to cleave oligos from solid support and to remove protecting groups globally. The oligo crude was then concentrated and purified by strong anion exchange or reverse phase HPLC. The purification fractions were combined and concentrated.

In some cases, oligo single strands were then conjugated with targeting ligands (e.g., sugar, peptides, antibodies) through post-synthetic conjugation to afford conjugates. The conjugation reactions were performed using standard conjugation methods. The conjugate crude was further purified by strong anion exchange or reverse phase HPLC. The purification fractions were combined and concentrated. The synthesized single strands were then dialyzed against water, concentrated, and their OD amounts were measured. The sense and antisense strands were annealed based on 1:1 molar ratio. The solution of the annealed duplex was lyophilized to afford the desired siRNA molecules.

To measure the in vitro activity, all test articles in the optimization series were transfected into Huh7 cells with Lipofectamine RNAiMax. In a 96-well plate, 20,000 cells/well were transfected with each compound with concentrations ranging from 5000 pM to 2 pM, according to the manufacturer's protocol (Invitrogen-13778-150). All transfections were carried out in triplicates. Prior to RNA isolation, cells were incubated at 37° C. for 24 hr. Then intracellular RNA was isolated with RNeasy 96 kit according to the manufacturer's protocol (Qiagen-74182). The target gene 1 cDNA was detected by qPCR and GAPDH cDNA was used an internal control and detected in parallel. GraphPad Prism was used to plot the graphs and calculate the IC₅₀s.

In the study described herein, the positional effects of 2′-F modification and 2′-OMe modifications of sense strand and antisense strands of a siRNA molecule against a target gene 1, on its stability and tolerability was examined.

Test of Chemical Modifications of Prototype siRNA Molecules of 21/23Mer to Target Gene 1

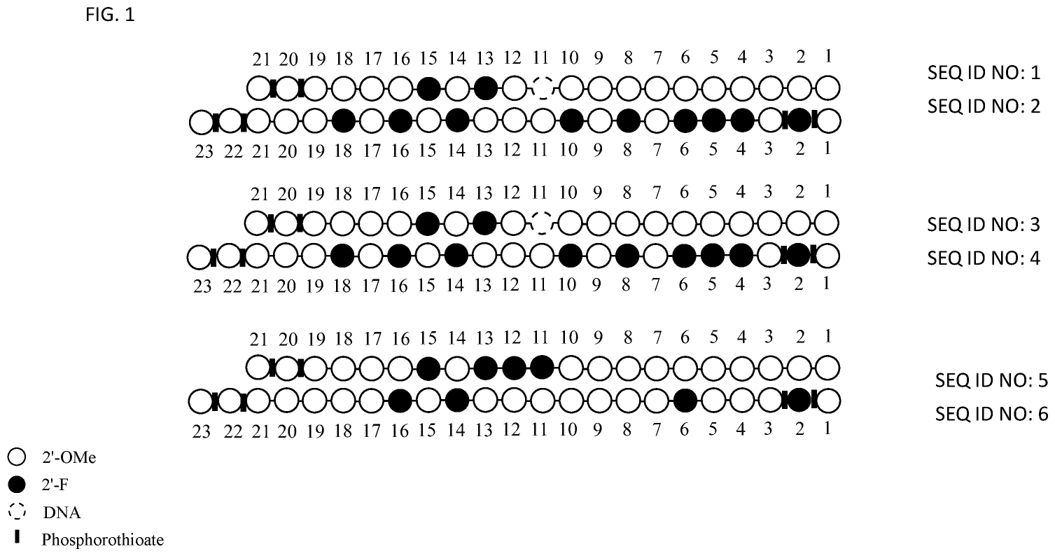

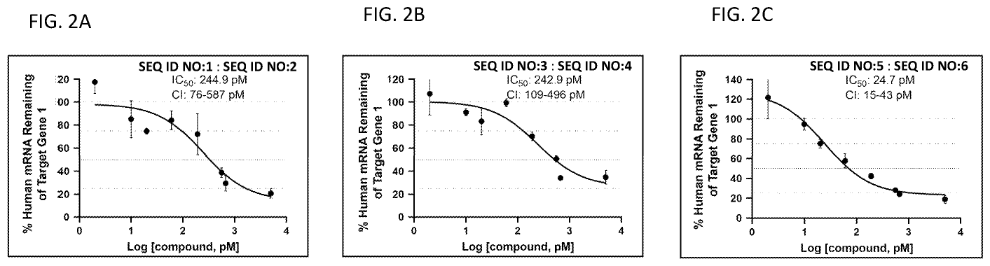

In the first set of optimization described herein (FIG. 1), the modification pattern of an exemplary siRNA prototype compound to target gene 1 was utilized as the parental design [SEQ ID NO: 1: SEQ ID NO: 2], which inhibited the target gene 1 with an IC₅₀ of 245 pm. Further modified siRNA molecules [SEQ ID NO: 3: SEQ ID NO: 4] showed a comparable IC₅₀ of 243 pm, and [SEQ ID NO: 5: SEQ ID NO: 6] showed a ˜10-fold increase of RNAi activity compared with that of the parental design (IC₅₀s of 25 pm vs 245 pm) (FIGS. 2A-2C).

Subsequent Optimization of Prototype siRNA Molecules: Positional Effect of 2′-OMe and 2′-F Modifications of Sense Strand

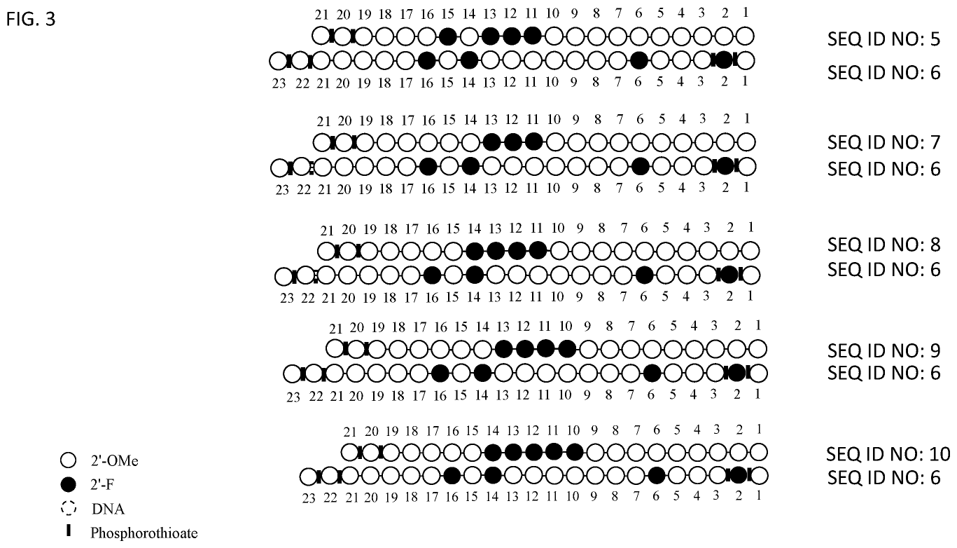

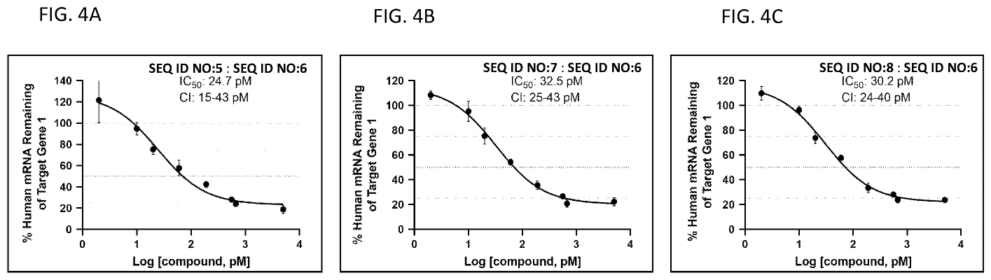

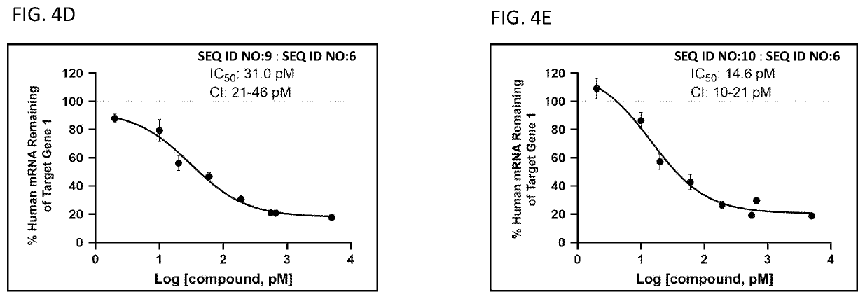

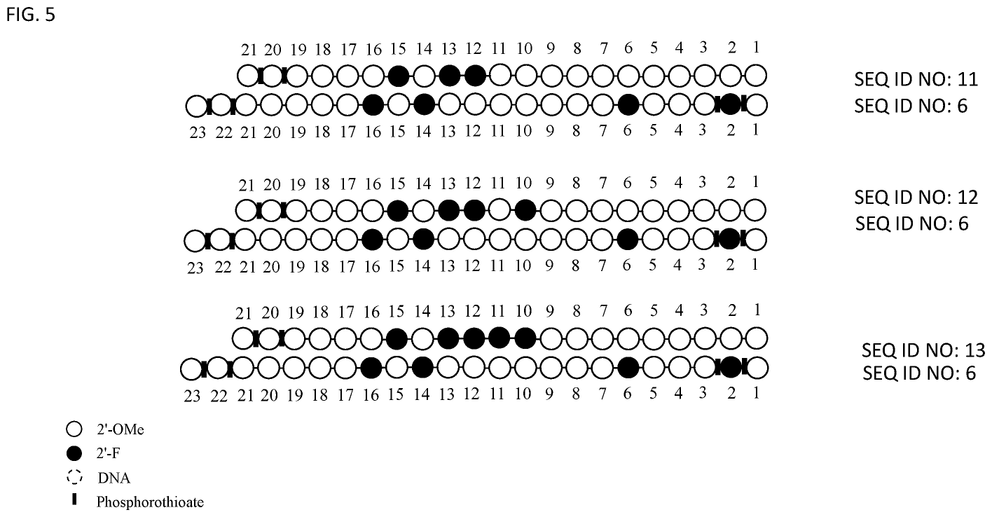

In a subsequent set of optimization described herein (FIGS. 3 and 5), improvement of potency (IC₅₀s) via refinement of the modification content (2′-OMe and 2′-F) on the sense strand was determined, with a focus on the positions ranging from 10 to 15. As shown in FIGS. 4A-4E, the in vitro activities of the siRNA molecules [SEQ ID NO: 7: SEQ ID NO: 6], [SEQ ID NO: 8: SEQ ID NO: 6], and [SEQ ID NO: 9: SEQ ID NO: 6] were well maintained with IC₅₀s of 33, 30, and 31 pm, respectively. However, [SEQ ID NO: 10: SEQ ID NO: 6] showed an IC₅₀ of 14 pm, which was a ˜2-fold improvement of potency compared with that of the siRNA molecule [SEQ ID NO: 7 and SEQ ID NO: 6], and also a ˜2-fold improvement compared with the siRNA molecule [SEQ ID NO: 5: SEQ ID NO: 6]. The results described herein indicated that 2′-F modification on positions 10 and 14, in combination with neighboring modifications, can additively improve in vitro activity.

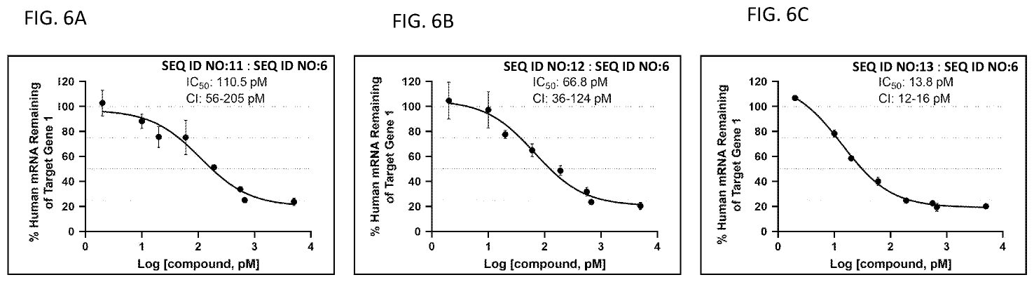

Further, when the 2′-OMe modification was moved to position 11 of the siRNA molecule [SEQ ID NO: 11: SEQ ID NO: 6] (FIG. 5), the in vitro potency was substantially compromised to afford an IC₅₀ of 110 pm, as shown in FIGS. 6A-6C. However, further modification on the siRNA molecule [SEQ ID NO: 12: SEQ ID NO: 6] was shown to partially rescue the potency with an IC₅₀ of 67 pm. Moreover, the siRNA molecule [SEQ ID NO: 13: SEQ ID NO: 6] showed an excellent IC₅₀ of 14 pm and a ˜2-fold improvement of in vitro activity compared with that of [SEQ ID NO: 5 and SEQ ID NO: 6].

Further Optimization of 2′-F Content of Antisense Strand of siRNA Molecules

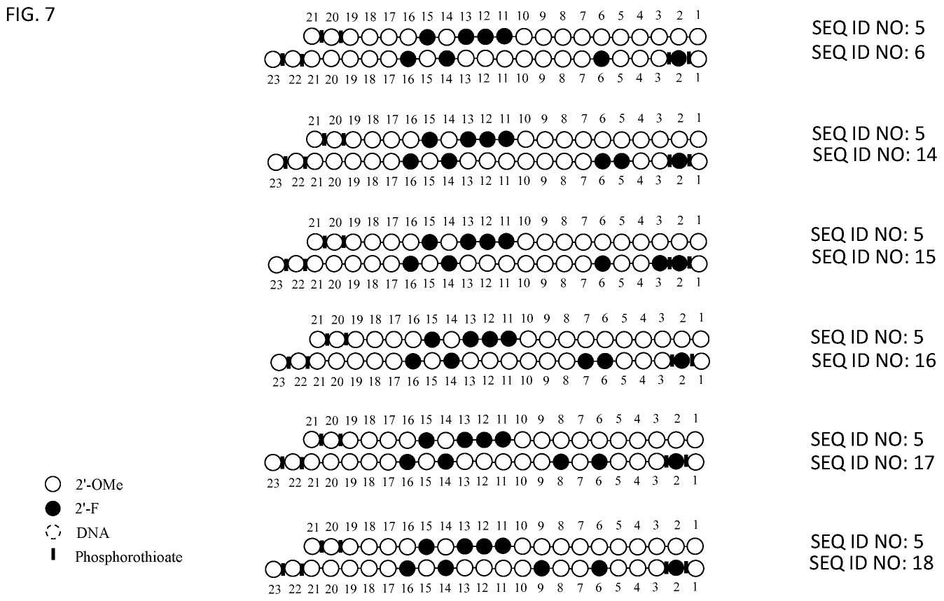

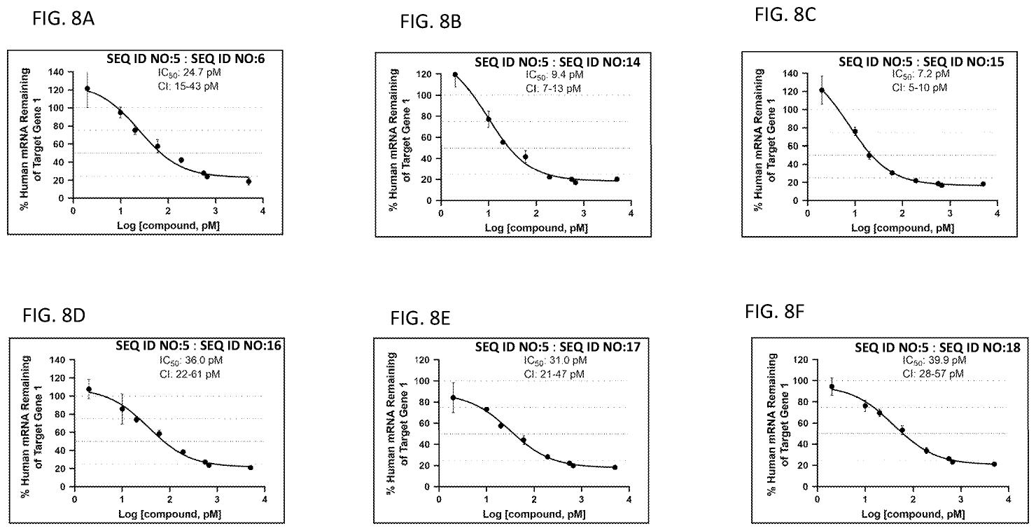

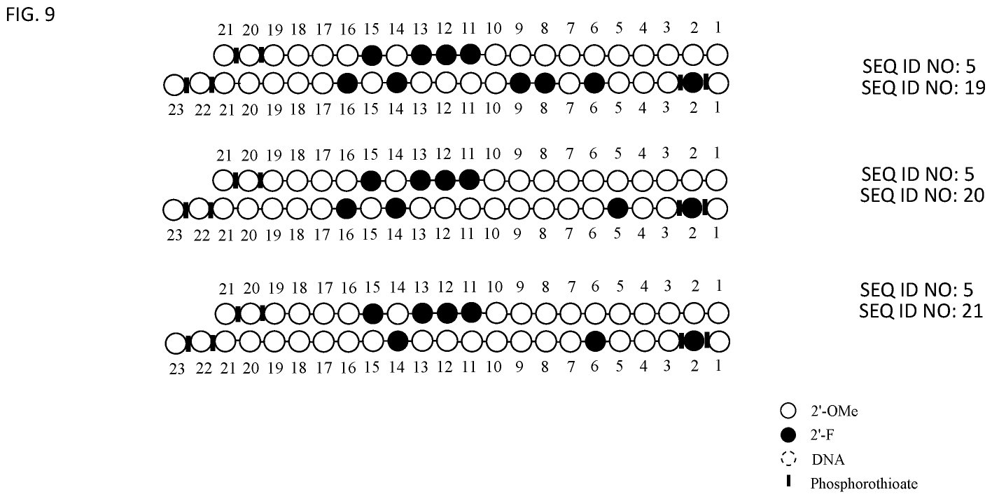

The results described herein describe optimizing the 2′-F content on the antisense strand to generate a third set of siRNAs (FIGS. 7 and 9). The design of siRNA comprising a sense strand and an antisense strand according to the siRNA molecule [SEQ ID NO: 5: SEQ ID NO: 6] was used as the parental design and examined for the impact of 2′-F modification mainly on the seed region (positions 2-8). The 2′-F modification was added on the positions 3, 5, 7, 8, and 9, respectively. Among these individual modifications, siRNA molecules [SEQ ID NO: 5: SEQ ID NO: 15] and [SEQ ID NO: 5: SEQ ID NO: 14] demonstrated IC₅₀s of 7 pm and 9 pm, which were 2-3-fold improvements of activity compared with that of parental design, as shown in FIGS. 8A-8F. The remaining modified siRNA molecules [SEQ ID NO: 5: SEQ ID NO: 16], [SEQ ID NO: 5: SEQ ID NO: 17], and [SEQ ID NO: 5: SEQ ID NO: 18] also showed marginal impact on the in vitro activity.

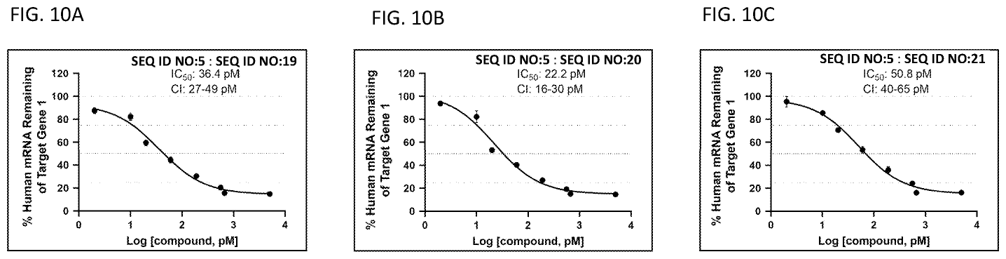

The combinational effects of 2′-F modification on some of these critical positions as shown in FIG. 9, was further examined. [SEQ ID NO: 5: SEQ ID NO: 19], [SEQ ID NO: 5: SEQ ID NO: 20], and [SEQ ID NO: 5: SEQ ID NO: 21] did not further improve in vitro activity compared with the parental design of the siRNA molecule [SEQ ID NO: 5: SEQ ID NO: 6]. See FIGS. 10A-10C.

### Fourth Optimization of Combined Modification on Sense and Antisense Strands

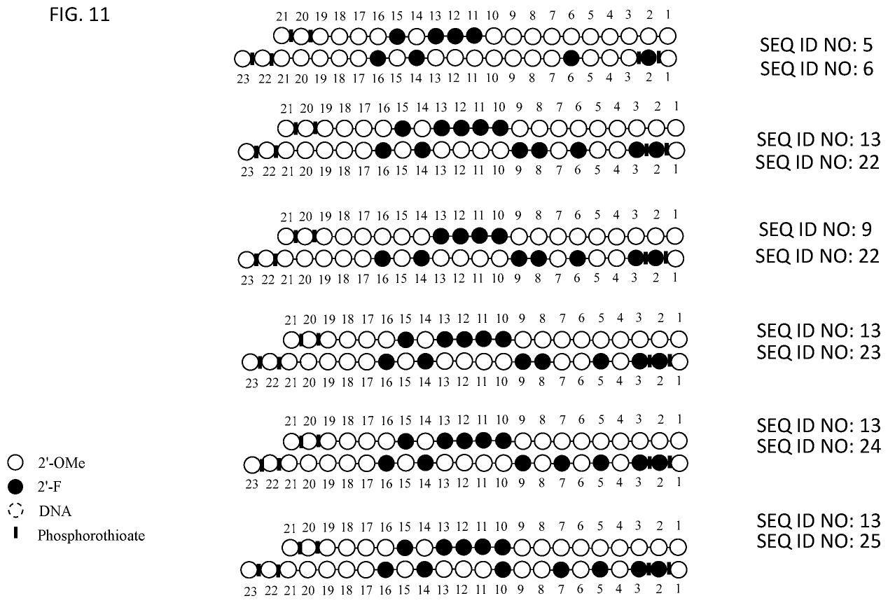

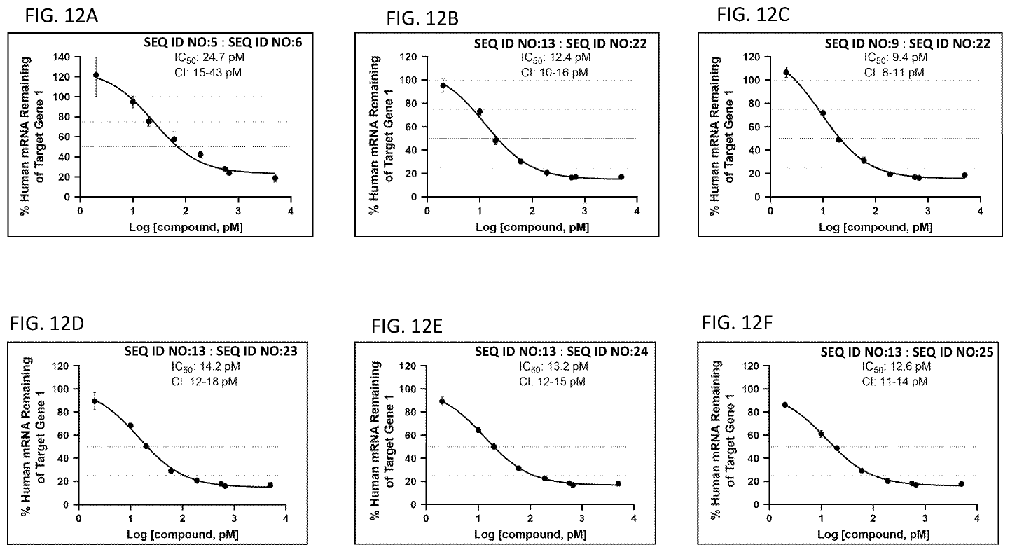

Described herein is a subsequent set of optimizations, the antisense with further optimized 2′-F content to afford antisense strand according to SEQ ID NO: 22, SEQ ID NO: 23, SEQ ID NO: 24, and SEQ ID NO: 25. The antisense strands were then paired with the optimal sense strand according to SEQ ID NO: 13 or the suboptimal sense according to SEQ ID NO: 9 identified (FIGS. 7 and 8A-8F), to examine their overall impact on the potency (FIG. 11). As shown in FIGS. 12A-12F, the siRNA molecule [SEQ ID NO: 9 and SEQ ID NO: 22] afforded an IC₅₀ of 9 pm, which was a ˜3-fold increase of in vitro activity compared with that of the siRNA molecule [SEQ ID NO: 9 and SEQ ID NO: 6], (IC₅₀ of 31 pm) and a ˜3-fold improvement compared with that of the parental design [SEQ ID NO: 5: SEQ ID NO: 6] (IC₅₀ of 25 pm). Paring the optimal sense strand according to SEQ ID NO: 13 with further optimized antisense strand according to SEQ ID NO: 22, SEQ ID NO: 23, SEQ ID NO: 24, and SEQ ID NO: 25 all showed excellent potency with IC₅₀s of 12-14 pm.

Optimization of Chemical Modifications on 20/22Mer to Improve the Stability and Potency of siRNAs Targeting Gene 1

Guided by the results of 2′-F and 2′-OMe modification in the 21/23mer platform described above, the positional effects in a 20/22mer platform following the same siRNA modification designs were examined. To generate the 20/22mer duplex based on the 21/23mer, both nucleosides were clipped on position 21 of the sense strand according to SEQ ID NO: 1 and position 23 of the antisense strand according to SEQ ID NO: 2 to afford a design comprising a sense and an antisense strand according to the siRNA molecule [SEQ ID NO: 26: SEQ ID NO: 27]. Therefore, [SEQ ID NO: 26: SEQ ID NO: 27] was designed as the 20/22mer counterpart of the 21/23mer [SEQ ID NO: 1: SEQ ID NO: 2], and used as the parental design in the following optimization of the 20/22mer platform. Although the number of the nucleoside position on the sense strand of 20mer is one less than that of the corresponding position on the sense strand of 21mer, substantial siRNA modification trends and positional effects that were observed in the 21/23mer platform could be translated to the 20/22mer platform.

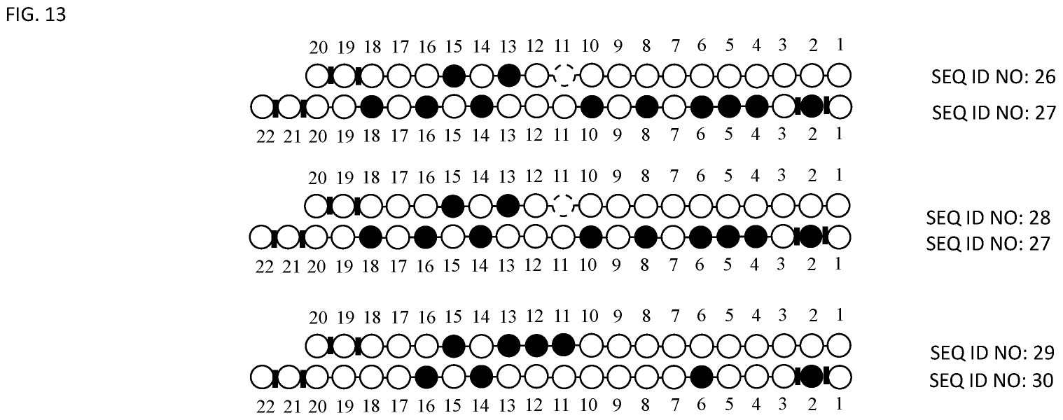

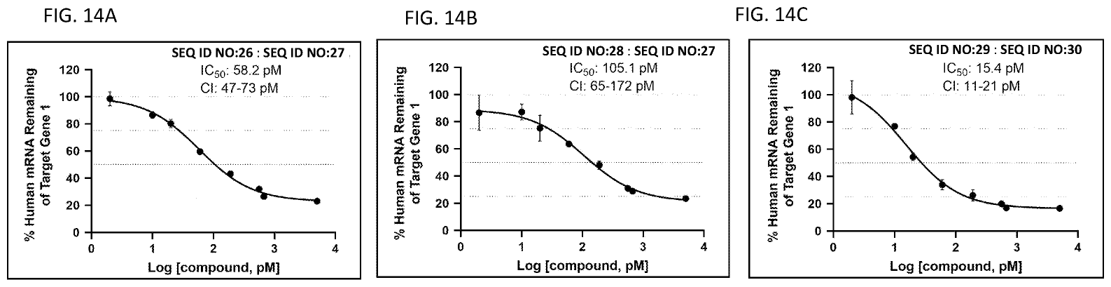

In the study described herein, the 1st siRNA modification set of the 20/22mer platform, [SEQ ID NO: 26: SEQ ID NO: 27] was used as the parental design, which demonstrated an IC₅₀ of 58 pm (FIG. 13). The modified siRNA molecule [SEQ ID NO: 28: SEQ ID NO: 27], demonstrated comparable potency as shown in FIGS. 14A-14C. Further chemically modified siRNA molecule [SEQ ID NO: 29: SEQ ID NO: 30] showed an IC₅₀ of 15 pm, which is a ˜4-fold improvement of RNAi activity compared with that of [SEQ ID NO: 26: SEQ ID NO: 27]. The significant improvement of in vitro activity was consistent with the trend observed with [SEQ ID NO: 5: SEQ ID NO: 6] when the same chemical modifications were applied to the 21/23mer sequences.

### Second Modification of 20/22Mer: Positional Effect of 2′-OMe and 2′-F Modifications of Sense Strand

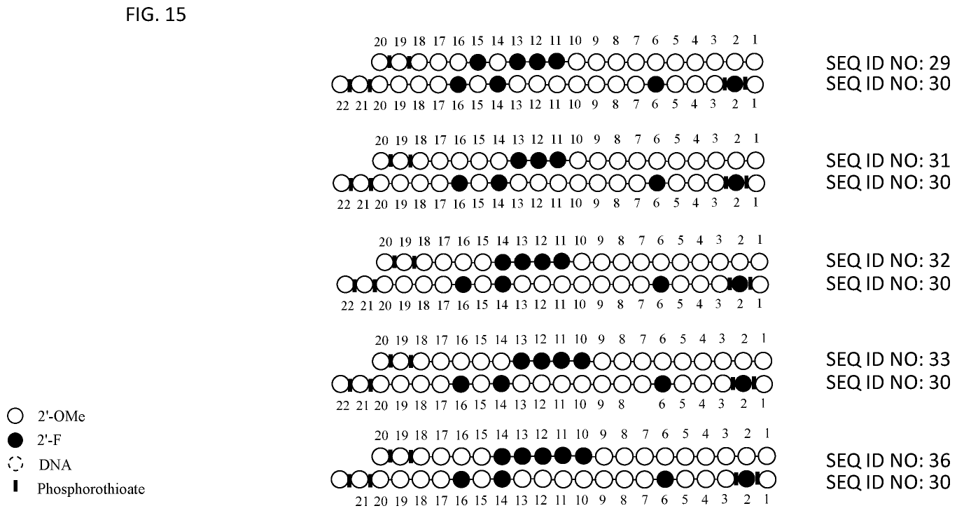

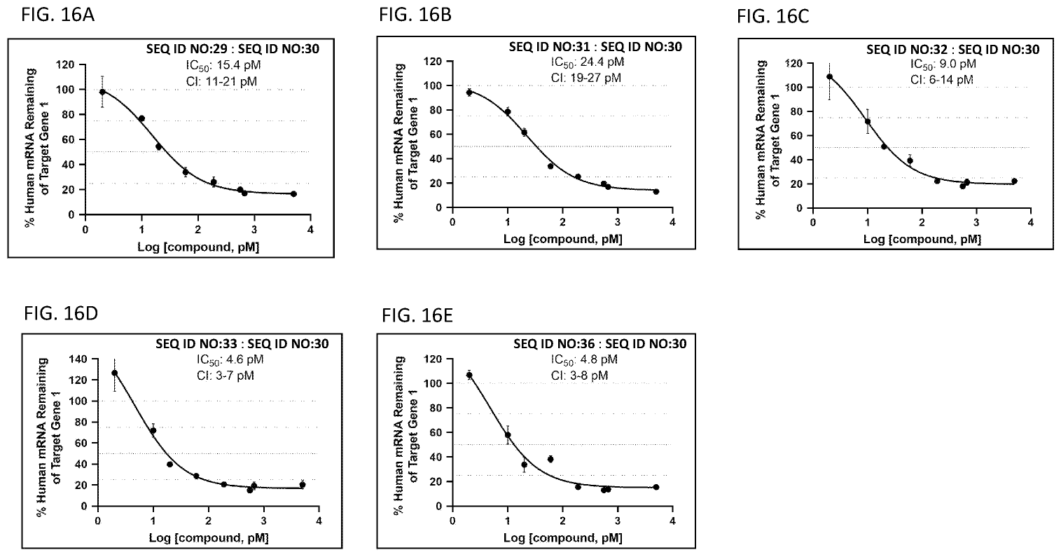

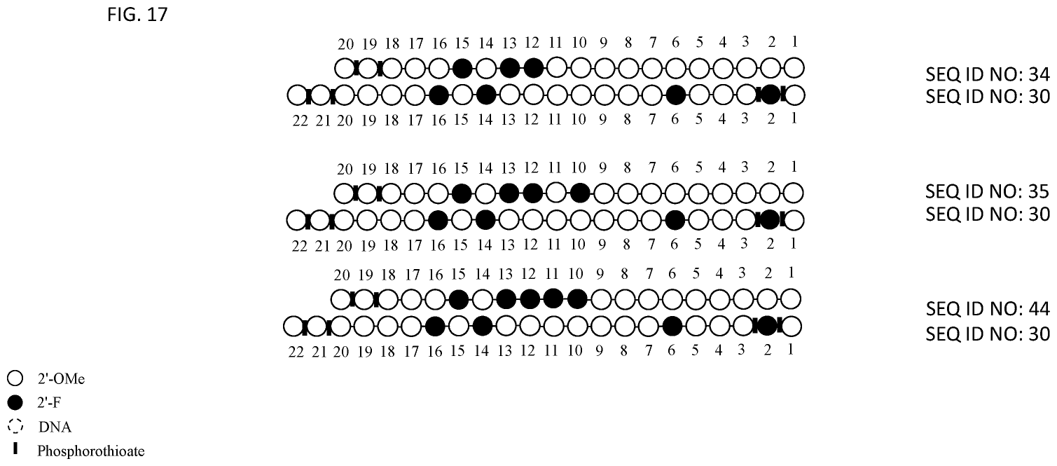

In the study described herein, the 2′-OMe content on the sense strand 2nd SAR set was refined (FIGS. 15 and 17). The study described herein identified that 2′-OMe modification on position 15 can well maintain the in vitro potency, regardless of the modification on position 14 [SEQ ID NO: 31: SEQ ID NO: 30] or [SEQ ID NO: 32: SEQ ID NO: 30]. The siRNA molecule [SEQ ID NO: 33: SEQ ID NO: 30] and [SEQ ID NO: 36: SEQ ID NO: 30] showed excellent IC₅₀s of 4-5 pm (FIGS. 16A-16E). These combinational effects of modification on positions 10, 14, and 15, on the sense strand of 20mer were consistent with those on the sense strand of 21mer.

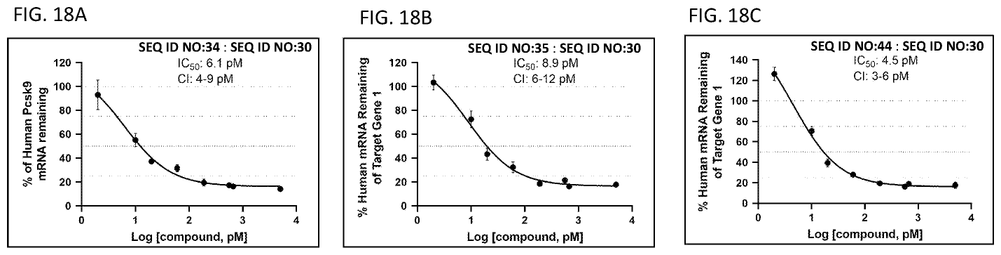

Further, the siRNA molecules [SEQ ID NO: 34: SEQ ID NO: 30], [SEQ ID NO: 35: SEQ ID NO: 30], and [SEQ ID NO: 44: SEQ ID NO: 30] all showed excellent IC₅₀s of 5-10 pm as shown in FIGS. 18A-18C.

### Third Modification of 20/22Mer: Positional Effects of 2′-OMe and 2′-F Modifications of Antisense Strand

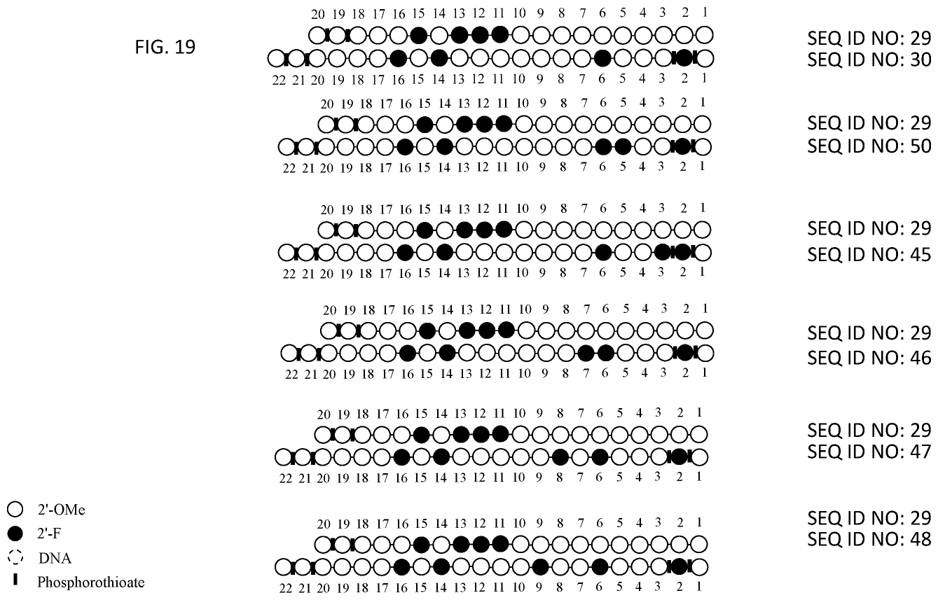

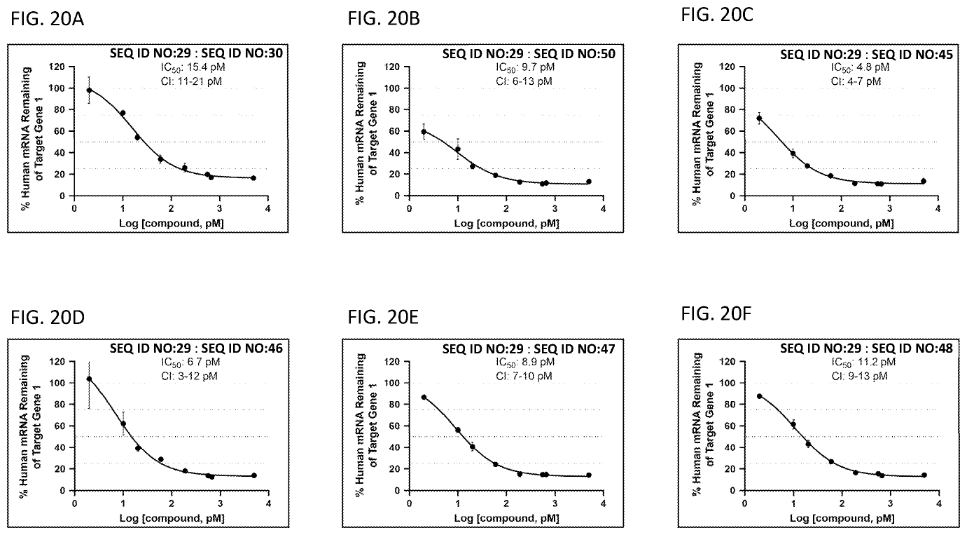

Described herein is generating of a 3rd set of siRNA modification to refine the 2′-F content on antisense strand for improved potency (FIG. 19). In the results described herein, it was shown that position 3 of the siRNA molecule [SEQ ID NO: 29: SEQ ID NO: 45] showed an IC₅₀ of 5 pm. 2′-F modification on other positions in the seed region, including positions 3 of the siRNA molecule [SEQ ID NO: 29: SEQ ID NO: 50], 5 of the siRNA molecule [SEQ ID NO: 29: SEQ ID NO: 46], 7 of the siRNA molecule [SEQ ID NO: 29: SEQ ID NO: 47], 8 of the siRNA molecule [SEQ ID NO: 29: SEQ ID NO: 47], and 9 of the siRNA molecule [SEQ ID NO: 29: SEQ ID NO: 48], also showed positive impact on the in vitro activity with IC₅₀s ranging from 7 to 11 pm (FIGS. 20B and 20D-20F).

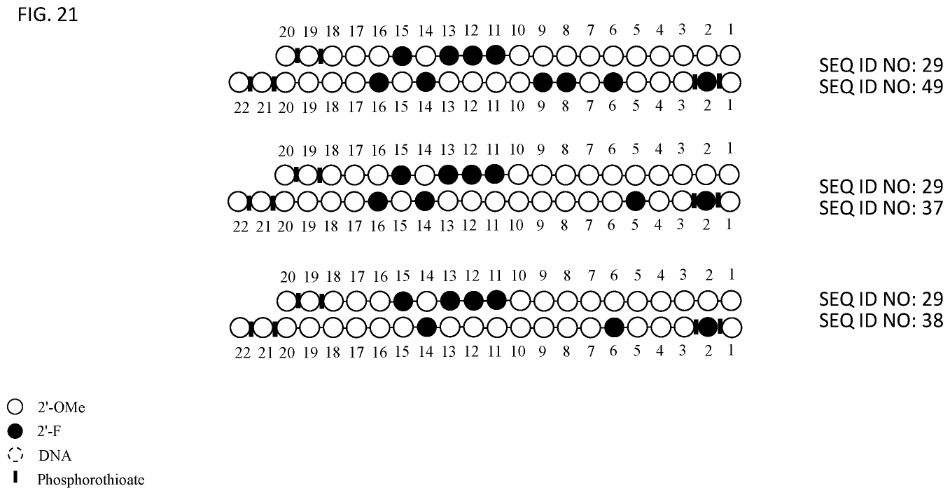

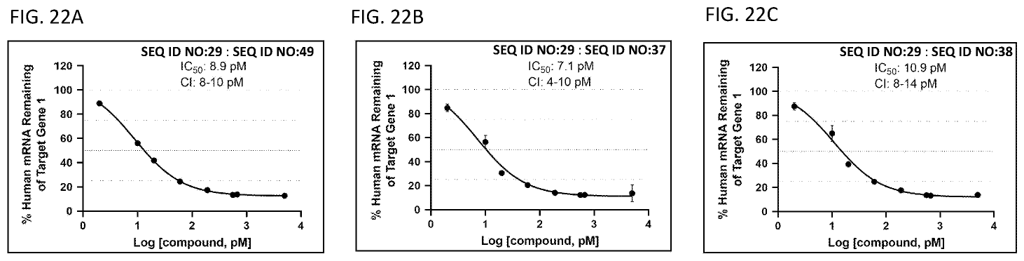

Further, the combinational effects of 2′-F modification on some of these critical positions in the seed region was examined (FIG. 21). As shown in FIGS. 22A-22C, the siRNA molecule [SEQ ID NO: 29: SEQ ID NO: 37] demonstrated 2-fold improvements of potency, compared with that of the parental design [SEQ ID NO: 29: SEQ ID NO: 30]. Also, the intrinsic potency of siRNA molecule [SEQ ID NO: 29: SEQ ID NO: 38] was well maintained.

Fourth Modification of 20/22Mer: Combinational Effect of the Optimized Sense with the Optimized (or Further Optimized) Antisense Strands

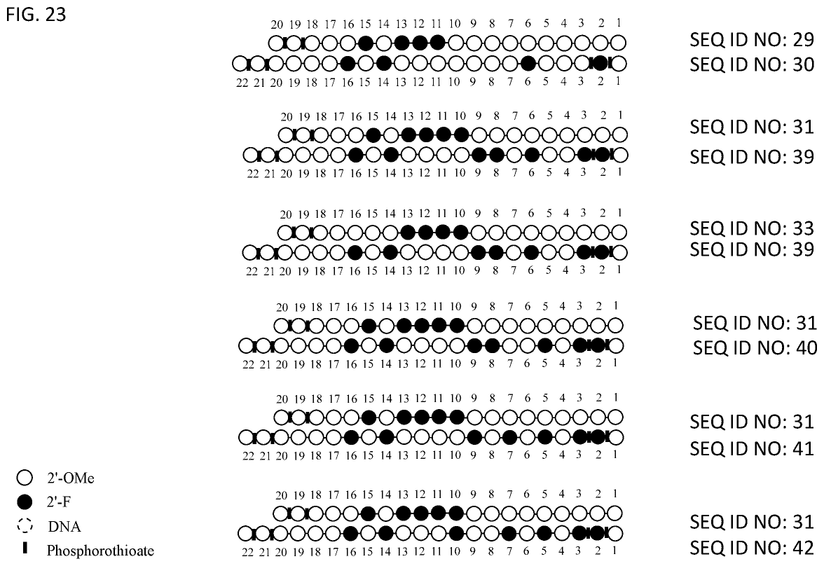

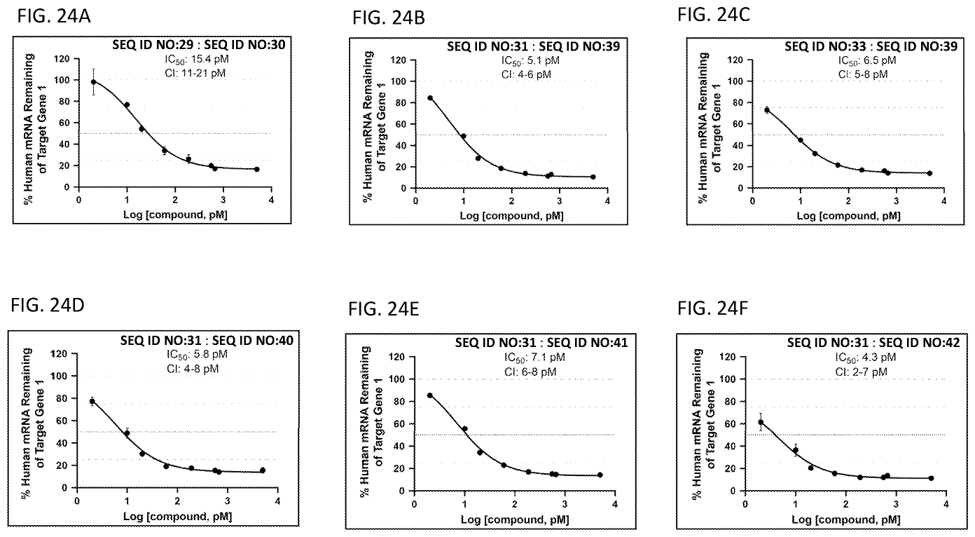

Described herein designing of a 4th set of siRNA modification to examine the combinational effect of the optimized sense with the optimized (or further optimized) antisense strands (FIG. 23). In the results described herein, all dual-optimized duplexes showed IC₅₀s <7 pm, which were shown to be improved by 2-fold of potency compared with the parental design (FIGS. 24A-24F). Among these molecules, the design comprising a sense strand and an antisense strand of the siRNA molecule [SEQ ID NO: 44: SEQ ID NO: 42], showed the lowest IC₅₀ of 4 pm, which is ˜60-fold improvement of in vitro activity compared with that of the parent siRNA molecule [SEQ ID NO: 1: SEQ ID NO: 2] with an IC₅₀ of 245 pm.

### Summary

In the results described herein, in both platforms of 21/23mer and 20/22mer, the 2′-O-methyl and 2′-fluoro content across the sense and antisense strands of siRNA molecules were systematically refined and the advanced modification design in terms of improvement of in vitro activity and stability were identified. The improved in vitro activity and stability disclosed herein can be translated to enhanced potency, duration, and tolerability in vivo.

The studies described herein also showed that paring of the optimized sense and antisense strands conferred the combinational improvement of activity, that led to the identification of several advanced designs e.g., [SEQ ID NO: 44: SEQ ID NO: 42] with up to 60-fold improvement of in vitro activity compared with that of a prototype benchmark.

### ADDITIONAL EMBODIMENTS

Additional embodiments of the disclosure include the following:
Embodiment 1. An isolated oligonucleotide, comprising:
   (a) a sense strand comprising X₁ nucleotides, wherein at least one nucleotide is modified with a first modification,
   each of the remaining nucleotides is independently modified with a second modification, and
   X₁ is an integer selected from 13-36,
   wherein the first modification and the second modification are different; and
   (b) an antisense strand comprising X₂ nucleotides, wherein at least one nucleotide is modified with a third modification,
   each of the remaining nucleotides is independently modified with a fourth modification, and
   X₂ is an integer selected from 18-31,
   wherein the third modification and the fourth modification are different.
Embodiment 2. The isolated oligonucleotide of embodiment 1, wherein the first modification is modification of the sugar moiety of the at least one nucleotide at the 2′-position selected from 2′-F modification, 2′-CN modification, 2′-N3 modification, 2′-deoxy modification, and an equivalent thereof, and a combination thereof.
Embodiment 3. The isolated oligonucleotide of embodiment 1 or 2, wherein the first modification is 2′-F modification, 2′-CN modification, 2′-N3 modification, or 2′-deoxy modification, or a stereoisomer thereof.
Embodiment 4. The isolated oligonucleotide of any one of embodiments 1-3, wherein the first modification is 2′-F modification, 2′-CN modification, or 2′-N3 modification, or a stereoisomer thereof.
Embodiment 5. The isolated oligonucleotide of any one of embodiments 1-4, wherein the first modification is 2′-F modification or a stereoisomer thereof.
Embodiment 6. The isolated oligonucleotide of any one of embodiments 1-5, wherein the second modification is modification of the sugar moiety of one or more of the remaining nucleotides at the 2′-position selected from 2′-C₁-C₆ alkyl, 2′-OR modification wherein R is C₁-C₆ alkyl optionally substituted with C₁-C₆ alkoxy, acetamide, phenyl, or heteroaryl comprising a 5- or 6-membered ring and 1 or 2 heteroatoms selected from N, O, and S, 2′-amino, and morpholino replacement, and an equivalent thereof, and a combination thereof.
Embodiment 7. The isolated oligonucleotide of any one of embodiments 1-6, wherein the second modification is 2′-OR modification.
Embodiment 8. The isolated oligonucleotide of any one of embodiments 1-7, wherein the second modification is 2′-O-methyl modification or 2′-methoxyethoxy modification.
Embodiment 9. The isolated oligonucleotide of any one of embodiments 1-8, wherein the second modification is 2′-O-methyl modification.
Embodiment 10. The isolated oligonucleotide of any one of embodiments 1-8, wherein the first modification is 2′-F modification or a stereoisomer thereof, and the second modification is 2′-O-methyl modification or 2′-methoxyethoxy modification.
Embodiment 11. The isolated oligonucleotide of any one of embodiments 1-10, wherein the first modification is 2′-F modification or a stereoisomer thereof, and the second modification is 2′-O-methyl modification.
Embodiment 12. The isolated oligonucleotide of any one of embodiments 1-11, wherein the third modification is modification of the sugar moiety of the at least one nucleotide at the 2′-position selected from 2′-F modification, 2′-CN modification, 2′-N3 modification, 2′-deoxy modification, and an equivalent thereof, and a combination thereof.
Embodiment 13. The isolated oligonucleotide of any one of embodiments 1-12, wherein the third modification is 2′-F modification, 2′-CN modification, 2′-N3 modification, or 2′-deoxy modification, or a stereoisomer thereof.
Embodiment 14. The isolated oligonucleotide of any one of embodiments 1-13, wherein the third modification is 2′-F modification, 2′-CN modification, or 2′-N3 modification, or a stereoisomer thereof.
Embodiment 15. The isolated oligonucleotide of any one of embodiments 1-14, wherein the third modification is 2′-F modification or a stereoisomer thereof.
Embodiment 16. The isolated oligonucleotide of any one of embodiments 1-15, wherein the fourth modification is modification of the sugar moiety of one or more of the remaining nucleotides at the 2′-position selected from 2′-C₁-C₆ alkyl, 2′-OR modification wherein R is C₁-C₆ alkyl optionally substituted with C₁-C₆ alkoxy, acetamide, phenyl, or heteroaryl comprising a 5- or 6-membered ring and 1 or 2 heteroatoms selected from N, O, and S, 2′-amino, and morpholino replacement, and an equivalent thereof, and a combination thereof.
Embodiment 17. The isolated oligonucleotide of any one of embodiments 1-16, wherein the fourth modification is 2′-OR modification.
Embodiment 18. The isolated oligonucleotide of any one of embodiments 1-17, wherein the fourth modification is 2′-O-methyl modification or 2′-methoxyethoxy modification.
Embodiment 19. The isolated oligonucleotide of any one of embodiments 1-18, wherein the fourth modification is 2′-O-methyl modification.
Embodiment 20. The isolated oligonucleotide of any one of embodiments 1-18, wherein the third modification is 2′-F modification or a stereoisomer thereof, and the fourth modification is 2′-O-methyl modification or 2′-methoxyethoxy modification.
Embodiment 21. The isolated oligonucleotide of any one of embodiments 1-20, wherein the third modification is 2′-F modification or a stereoisomer thereof, and the fourth modification is 2′-O-methyl modification.
Embodiment 22. The isolated oligonucleotide of any one of embodiments 1-21, wherein in the sense strand at least three nucleotides are modified with the first modification.
Embodiment 23. The isolated oligonucleotide of embodiment 22, wherein at least two of the at least three nucleotides modified with the first modification are consecutively located.
Embodiment 24. The isolated oligonucleotide of embodiment 22 or 23, wherein at least three of the at least three nucleotides modified with the first modification are consecutively located.
Embodiment 25. The isolated oligonucleotide of any one of embodiments 1-24, wherein in the sense strand at least four nucleotides are modified with the first modification.
Embodiment 26. The isolated oligonucleotide of embodiment 25, wherein at least three of the at least four nucleotides modified with the first modification are consecutively located.
Embodiment 27. The isolated oligonucleotide of embodiment 25 or 26, wherein at least four of the at least four nucleotides modified with the first modification are consecutively located.
Embodiment 28. The isolated oligonucleotide of any one of embodiments 1-27, wherein in the sense strand at least five nucleotides are modified with the first modification.
Embodiment 29. The isolated oligonucleotide of embodiment 28, wherein at least three of the at least five nucleotides modified with the first modification are consecutively located.
Embodiment 30. The isolated oligonucleotide of embodiment 28 or 29, wherein at least four of the at least five nucleotides modified with the first modification are consecutively located.
Embodiment 31. The isolated oligonucleotide of any one of embodiments 22-30, wherein the at least three nucleotides, the at least four nucleotides, or the at least five nucleotides modified with the first modification are located from position 10 to position 15 from the nucleotide complementary to the first nucleotide at the 5′-terminus of the antisense strand.
Embodiment 32. The isolated oligonucleotide of any one of embodiments 22-31, wherein two of the at least three nucleotides modified with the first modification are located at positions selected from position 10, 11, 12, and 13 from the nucleotide complementary to the first nucleotide at the 5′-terminus of the antisense strand.
Embodiment 33. The isolated oligonucleotide of any one of embodiments 22-32, wherein three of the at least three nucleotides modified with the first modification are located at positions selected from position 10, 11, 12, and 13 from the nucleotide complementary to the first nucleotide at the 5′-terminus of the antisense strand.
Embodiment 34. The isolated oligonucleotide of any one of embodiments 22-33, wherein one of the at least three nucleotides modified with the first modification is located at position 11 from the nucleotide complementary to the first nucleotide at the 5′-terminus of the antisense strand.
Embodiment 35. The isolated oligonucleotide of any one of embodiments 22-34, wherein one of the at least four nucleotides modified with the first modification is located at position 10 from the nucleotide complementary to the first nucleotide at the 5′-terminus of the antisense strand.
Embodiment 36. The isolated oligonucleotide of any one of embodiments 22-34, wherein one of the at least four nucleotides modified with the first modification is located at position 14 from the nucleotide complementary to the first nucleotide at the 5′-terminus of the antisense strand.
Embodiment 37. The isolated oligonucleotide of any one of embodiments 22-34, wherein two of the at least four nucleotides modified with the first modification are located from position 10 to position 14 from the nucleotide complementary to the first nucleotide at the 5′-terminus of the antisense strand.
Embodiment 38. The isolated oligonucleotide of any one of embodiments 22-37, wherein not all of the at least three nucleotides modified with the first modification are consecutively located.
Embodiment 39. The isolated oligonucleotide of any one of embodiments 22-38, wherein the at least three nucleotides, the at least four nucleotides, or the at least five nucleotides are modified with 2′-F modification.
Embodiment 40. The isolated oligonucleotide of embodiment 31, wherein in the sense strand at most three nucleotides are modified with the first modification at positions 12, 13 and 15, from the nucleotide complementary to the first nucleotide at the 5′-terminus of the antisense strand.
Embodiment 41. The isolated oligonucleotide of embodiment 31, wherein in the sense strand at most four nucleotides are modified with the first modification at positions 11, 12, 13 and 14, from the nucleotide complementary to the first nucleotide at the 5′-terminus of the antisense strand.
Embodiment 42. The isolated oligonucleotide of embodiment 31, wherein in the sense strand at most four nucleotides are modified with the first modification at positions 10, 11, 12 and 13, from the nucleotide complementary to the first nucleotide at the 5′-terminus of the antisense strand.
Embodiment 43. The isolated oligonucleotide of embodiment 31, wherein in the sense strand at most four nucleotides are modified with the first modification at positions 10, 12, 13 and 15, from the nucleotide complementary to the first nucleotide at the 5′-terminus of the antisense strand.
Embodiment 44. The isolated oligonucleotide of embodiment 31, wherein in the sense strand at most five nucleotides are modified with the first modification at positions 10, 11, 12, 13 and 15, from the nucleotide complementary to the first nucleotide at the 5′-terminus of the antisense strand.
Embodiment 45. The isolated oligonucleotide of embodiment 31, wherein in the sense strand at most five nucleotides are modified with the first modification at positions 10, 11, 12, 13 and 14, from the nucleotide complementary to the first nucleotide at the 5′-terminus of the antisense strand.
Embodiment 46. The isolated oligonucleotide of any one of embodiments 1-45, wherein the sense strand comprises nucleotides modified with 2′-F modification, and nucleotides modified with 2′-O-methyl modification, according to the formula:

5′ (M)_{g}(F)_{f}(M)ₑ(F)_{d}(M)_{c}(F)_{b}(M)ₐ 3′,

wherein M is 2′-O-methyl modified nucleotide, F is 2′-F modified nucleotide, and each of a, b, c, d, e, f, and g are any one of 0-16, and indicates the number of consecutive nucleotides modified with the modification, and wherein the sense strand is any one of:

| | a) |
|---|---|
| (SEQ ID NO: 52) | |
| | 5′(M)₀(F)₀(M)₀(F)₀(M)₈(F)₃(M)₁₀ 3′; |
| | b) |
| (SEQ ID NO: 53) | |
| | 5′(M)₀(F)₀(M)₅(F)₁(M)₁(F)₃(M)₁₀ 3′; |
| | c) |
| (SEQ ID NO: 54) | |
| | 5′(M)₀(F)₀(M)₀(F)₀(M)₇(F)₃(M)₁₀ 3′; |
| | d) |
| (SEQ ID NO: 55) | |
| | 5′(M)₀(F)₀(M)₅(F)₁(M)₁(F)₂(M)₁₁ 3′; |
| | e) |
| (SEQ ID NO: 56) | |
| | 5′(M)₀(F)₀(M)₆(F)₁(M)₁(F)₂(M)₁₁ 3′; |
| | f) |
| (SEQ ID NO: 57) | |
| | 5′(M)₆(F)₁(M)₁(F)₂(M)₁(F)₁(M)₉ 3′; |
| | g) |
| (SEQ ID NO: 58) | |
| | 5′(M)₅(F)₁(M)₁(F)₂(M)₁(F)₁(M)₉ 3′; |
| | h) |
| (SEQ ID NO: 59) | |
| | 5′(M)₀(F)₀(M)₀(F)₀(M)₇(F)₄(M)₁₀ 3′; |
| | i) |
| (SEQ ID NO: 60) | |
| | 5′(M)₀(F)₀(M)₆(F)₁(M)₁(F)₄(M)₉ 3′; |
| | j) |
| (SEQ ID NO: 61) | |
| | 5′(M)₀(F)₀(M)₀(F)₀(M)₈(F)₄(M)₉ 3′; |
| | k) |
| (SEQ ID NO: 62) | |
| | 5′(M)₀(F)₀(M)₀(F)₀(M)₆(F)₄(M)₁₀ 3′; |
| | l) |
| (SEQ ID NO: 63) | |
| | 5′(M)₀(F)₀(M)₅(F)₁(M)₁(F)₄(M)₉ 3′; |
| | m) |
| (SEQ ID NO: 64) | |
| | 5′(M)₀(F)₀(M)₀(F)₀(M)₇(F)₄(M)₉ 3′; |
| | n) |
| (SEQ ID NO: 65) | |
| | 5′(M)₀(F)₀(M)₀(F)₀(M)₇(F)₅(M)₉ 3′; |
| | and |
| | o) |
| (SEQ ID NO: 66) | |
| | 5′(M)₀(F)₀(M)₀(F)₀(M)₆(F)₅(M)₉ 3′. |

Embodiment 47. The isolated oligonucleotide of embodiment 1-46, wherein in the antisense strand at most seven nucleotides are modified with the third modification.
Embodiment 48. The isolated oligonucleotide of embodiment 47, wherein at most four of the at most seven nucleotides modified with the third modification are located from position 2 to position 8 from the first nucleotide at the 5′-terminus of the antisense strand.
Embodiment 49. The isolated oligonucleotide of embodiment 47 or 48, wherein at most two of the at most seven nucleotides modified with the third modification are consecutively located.
Embodiment 50. The isolated oligonucleotide of any one of embodiments 47-49, wherein three or four of the at most seven nucleotides modified with the third modification are located at positions selected from position 2, 3, 5, 6, 7, and 8 from the first nucleotide at the 5′-terminus of the antisense strand.
Embodiment 51. The isolated oligonucleotide of any one of embodiments 47-50, wherein at least one of the at most seven nucleotides modified with the third modification is located at position 14 from the first nucleotide at the 5′-terminus of the antisense strand.
Embodiment 52. The isolated oligonucleotide of any one of embodiments 47-51, wherein two or three of the at most seven nucleotides modified with the third modification are located at positions selected from position 2, 3, 5, and 6 from the first nucleotide at the 5′-terminus of the antisense strand.
Embodiment 53. The isolated oligonucleotide of any one of embodiments 47-52, wherein three of the at most seven nucleotides modified with the third modification are located at positions selected from position 2, 3, 5, and 6 from the first nucleotide at the 5′-terminus of the antisense strand.
Embodiment 54. The isolated oligonucleotide of any one of embodiments 47-53, wherein two of the at most seven nucleotides modified with the third modification are located at positions 8 and 14 from the nucleotide complementary to the first nucleotide at the 5′-terminus of the antisense strand.
Embodiment 55. The isolated oligonucleotide of any one of embodiments 47-54, wherein one or two of the at most seven nucleotides modified with the third modification are located at positions selected from position 14 and 16 from the first nucleotide at the 5′-terminus of the antisense strand.
Embodiment 56. The isolated oligonucleotide of any one of embodiments 47-55, wherein two of the at most seven nucleotides modified with the third modification are located at positions 14 and 16 from the first nucleotide at the 5′-terminus of the antisense strand.
Embodiment 57. The isolated oligonucleotide of any one of embodiments 47-56, wherein the at most seven nucleotides are modified with 2′-F modification.
Embodiment 58. The isolated oligonucleotide of any one of embodiments 47-57, wherein at most six nucleotides are modified with the third modification.
Embodiment 59. The isolated oligonucleotide of any one of embodiments 47-58, wherein the at most six nucleotides are modified with 2′-F modification.
Embodiment 60. The isolated oligonucleotide of any one of embodiments 47-59, wherein at most five nucleotides are modified with the third modification.
Embodiment 61. The isolated oligonucleotide of any one of embodiments 47-60, wherein the at most five nucleotides are modified with 2′-F modification.
Embodiment 62. The isolated oligonucleotide of any one of embodiments 47-61, wherein at most four nucleotides are modified with the third modification.
Embodiment 63. The isolated oligonucleotide of any one of embodiments 47-62, wherein at most four nucleotides are modified with 2′-F modification.
Embodiment 64. The isolated oligonucleotide of any one of embodiments 47-63, wherein at most three nucleotides are modified with the third modification.
Embodiment 65. The isolated oligonucleotide of any one of embodiments 47-64, wherein the at least three nucleotides are modified with 2′-F modification.
Embodiment 66. The isolated oligonucleotide of any one of embodiments 60-61, wherein in the antisense strand at most five nucleotides are modified with the third modification at positions 2, 3, 6, 14 and 16, from the nucleotide complementary to the first nucleotide at the 5′-terminus of the antisense strand.
Embodiment 67. The isolated oligonucleotide of any one of embodiments 60-61, wherein in the antisense strand at most five nucleotides are modified with the third modification at positions 2, 6, 7, 14 and 16, from the nucleotide complementary to the first nucleotide at the 5′-terminus of the antisense strand.
Embodiment 68. The isolated oligonucleotide of any one of embodiments 58-59, wherein in the antisense strand at most six nucleotides are modified with the third modification at positions 2, 3, 6, 8, 9 and 16, from the nucleotide complementary to the first nucleotide at the 5′-terminus of the antisense strand.
Embodiment 69. The isolated oligonucleotide of any one of embodiments 58-59, wherein in the antisense strand at most six nucleotides are modified with the third modification at positions 2, 3, 5, 8, 9 and 16, from the nucleotide complementary to the first nucleotide at the 5′-terminus of the antisense strand.
Embodiment 70. The isolated oligonucleotide of any one of embodiments 47-57, wherein in the antisense strand at most seven nucleotides are modified with the third modification at positions 2, 3, 5, 7, 9, 14 and 16, from the nucleotide complementary to the first nucleotide at the 5′-terminus of the antisense strand.
Embodiment 71. The isolated oligonucleotide of any one of embodiments 47-57, wherein in the antisense strand at most seven nucleotides are modified with the third modification at positions 2, 3, 5, 7, 10, 14 and 16, from the nucleotide complementary to the first nucleotide at the 5′-terminus of the antisense strand.
Embodiment 72. The isolated oligonucleotide of any one of embodiments 1-71, wherein the antisense strand comprises nucleotides modified with 2′-F modification, and nucleotides modified with 2′-O-methyl modification, according to the formula:

3′ (M)ₐ(F)_{b}(M)_{c}(F)_{d}(M)ₑ(F)_{f}(M)_{g}(F)ₕ(M)ᵢ(F)ⱼ(M)ₖ
(F)ₗ(M)ₘ(F)ₙ(M)ₒ 5′,

wherein M is 2′-O-methyl modified nucleotide, F is 2′-F modified nucleotide, and each of a, b, c, d, e, f, g, h, i, j, k, l, m, n, and o are any one of 0-16, and indicates the number of consecutive nucleotides modified with the modification, and wherein the antisense strand is any one of:

1)
(SEQ ID NO: 14)
3′(M)₀(F)₀(M)₀(F)₀(M)₀(F)₀(M)₇(F)₁(M)₁(F)₁(M)₇(F)₂(M)₂(F)₁(M)₁ 5′;
2)
(SEQ ID NO: 15)
3′(M)₀(F)₀(M)₀(F)₀(M)₀(F)₀(M)₇(F)₁(M)₁(F)₁(M)₇(F)₁(M)₂(F)₂(M)₁ 5′;
3)
(SEQ ID NO: 16)
3′(M)₀(F)₀(M)₀(F)₀(M)₀(F)₀(M)₇(F)₁(M)₁(F)₁(M)₆(F)₂(M)₃(F)₁(M)₁ 5′;
4)
(SEQ ID NO: 17)
3′(M)₀(F)₀(M)₀(F)₀(M)₇(F)₁(M)₁(F)₁(M)₅(F)₁(M)₁(F)₁(M)₃(F)₁(M)₁ 5′;
5)
(SEQ ID NO: 18)
3′(M)₀(F)₀(M)₀(F)₀(M)₇(F)₁(M)₁(F)₁(M)₄(F)₁(M)₂(F)₁(M)₃(F)₁(M)₁ 5′;
6)
(SEQ ID NO: 20)
3′(M)₀(F)₀(M)₀(F)₀(M)₀(F)₀(M)₇(F)₁(M)₁(F)₁(M)₈(F)₁(M)₂(F)₁(M)₁ 5′;
7)
(SEQ ID NO: 21)
3′(M)₀(F)₀(M)₀(F)₀(M)₀(F)₀(M)₀(F)₀(M)₉(F)₁(M)₇(F)₁(M)₃(F)₁(M)₁ 5′;
8)
(SEQ ID NO: 22)
3′(M)₀(F)₀(M)₀(F)₀(M)₇(F)₁(M)₁(F)₁(M)₄(F)₂(M)₁(F)₁(M)₂(F)₂(M)₁ 5′;
9)
(SEQ ID NO: 23)
3′(M)₀(F)₀(M)₀(F)₀(M)₇(F)₁(M)₁(F)₁(M)₄(F)₂(M)₂(F)₁(M)₁(F)₂(M)₁ 5′;
10)
(SEQ ID NO: 24)
3′(M)₀(F)₀(M)₀(F)₀(M)₇(F)₁(M)₁(F)₁(M)₄(F)₁(M)₁(F)₁(M)₁(F)₂(M)₁ 5′;
11)
(SEQ ID NO: 25)
3′(M)₀(F)₀(M)₀(F)₀(M)₇(F)₁(M)₁(F)₁(M)₃(F)₁(M)₂(F)₁(M)₁(F)₁(M)₁(F)₂(M)₁ 5′;
12)
(SEQ ID NO: 27)
3′(M)₀(F)₀(M)₄(F)₁(M)₁(F)₁(M)₁(F)₁(M)₃(F)₁(M)₁(F)₁(M)₁(F)₃(M)₁(F)₁(M)₁ 5′;
13)
(SEQ ID NO: 30)
3′(M)₀(F)₀(M)₀(F)₀(M)₀(F)₀(M)₆(F)₁(M)₁(F)₁(M)₇(F)₁(M)₃(F)₁(M)₁ 5′;
14)
(SEQ ID NO: 50)
3′(M)₀(F)₀(M)₀(F)₀(M)₀(F)₀(M)₆(F)₁(M)₁(F)₁(M)₇(F)₂(M)₂(F)₁(M)₁ 5′;
15)
(SEQ ID NO: 45)
3′(M)₀(F)₀(M)₀(F)₀(M)₀(F)₀(M)₆(F)₁(M)₁(F)₁(M)₇(F)₁(M)₂(F)₂(M)₁ 5′;
16)
(SEQ ID NO: 46)
3′(M)₀(F)₀(M)₀(F)₀(M)₀(F)₀(M)₆(F)₁(M)₁(F)₁(M)₆(F)₂(M)₃(F)₁(M)₁ 5′;
17)
(SEQ ID NO: 47)
3′(M)₀(F)₀(M)₀(F)₀(M)₆(F)₁(M)₁(F)₁(M)₅(F)₁(M)₁(F)₁(M)₃(F)₁(M)₁ 5′;
18)
(SEQ ID NO: 48)
3′(M)₀(F)₀(M)₀(F)₀(M)₆(F)₁(M)₁(F)₁(M)₄(F)₁(M)₂(F)₁(M)₃(F)₁(M)₁ 5′;
19)
(SEQ ID NO: 49)
3′(M)₀(F)₀(M)₀(F)₀(M)₆(F)₁(M)₁(F)₁(M)₄(F)₂(M)₁(F)₁(M)₃(F)₁(M)₁ 5′;
20)
(SEQ ID NO: 37)
3′(M)₀(F)₀(M)₀(F)₀(M)₀(F)₀(M)₆(F)₁(M)₁(F)₁(M)₈(F)₁(M)₂(F)₁(M)₁ 5′;
21)
(SEQ ID NO: 38)
3′(M)₀(F)₀(M)₀(F)₀(M)₀(F)₀(M)₀(F)₀(M)₈(F)₁(M)₇(F)₁(M)₃(F)₁(M)₁ 5′;
22)
(SEQ ID NO: 39)
3′(M)₀(F)₀(M)₀(F)₀(M)₆(F)₁(M)₁(F)₁(M)₄(F)₂(M)₁(F)₁(M)₂(F)₂(M)₁ 5′;
23)
(SEQ ID NO: 40)
3′(M)₀(F)₀(M)₀(F)₀(M)₆(F)₁(M)₁(F)₁(M)₄(F)₂(M)₂(F)₁(M)₁(F)₂(M)₁ 5′;
24)
(SEQ ID NO: 41)
3′(M)₀(F)₀(M)₆(F)₁(M)₁(F)₁(M)₄(F)₁(M)₁(F)₁(M)₁(F)₁(M)₁(F)₂(M)₁ 5′;
and
25)
(SEQ ID NO: 42)
3′(M)₀(F)₀(M)₆(F)₁(M)₁(F)₁(M)₃(F)₁(M)₂(F)₁(M)₁(F)₁(M)₁(F)₂(M)₁ 5′.

Embodiment 73. The isolated oligonucleotide of embodiment 72, wherein the sense strand comprises
   nucleotides modified with 2′-F modification, and nucleotides modified with 2′-O-methyl modification, according to the formula:

5′ (M)_{g}(F)_{f}(M)ₑ(F)_{d}(M)_{c}(F)_{b}(M)ₐ 3′,

Wherein M is 2′-O-methyl modified nucleotide, F is 2′-F modified nucleotide, and each of a, b, c, d, e, f, and g are any one of 0-16, and indicates the number of consecutive nucleotides modified with the modification, and wherein the sense strand is any one of:

| | a) |
|---|---|
| (SEQ ID NO: 52) | |
| | 5′(M)₀(F)₀(M)₀(F)₀(M)₈(F)₃(M)₁₀ 3′; |
| | b) |
| (SEQ ID NO: 53) | |
| | 5′(M)₀(F)₀(M)₅(F)₁(M)₁(F)₃(M)₁₀ 3′; |
| | c) |
| (SEQ ID NO: 54) | |
| | 5′(M)₀(F)₀(M)₀(F)₀(M)₇(F)₃(M)₁₀ 3′; |
| | d) |
| (SEQ ID NO: 55) | |
| | 5′(M)₀(F)₀(M)₅(F)₁(M)₁(F)₂(M)₁₁ 3′; |
| | e) |
| (SEQ ID NO: 56) | |
| | 5′(M)₀(F)₀(M)₆(F)₁(M)₁(F)₂(M)₁₁ 3′; |
| | f) |
| (SEQ ID NO: 57) | |
| | 5′(M)₆(F)₁(M)₁(F)₂(M)₁(F)₁(M)₉ 3′; |
| | g) |
| (SEQ ID NO: 58) | |
| | 5′(M)₅(F)₁(M)₁(F)₂(M)₁(F)₁(M)₉ 3′; |
| | h) |
| (SEQ ID NO: 59) | |
| | 5′(M)₀(F)₀(M)₀(F)₀(M)₇(F)₄(M)₁₀ 3′; |
| | i) |
| (SEQ ID NO: 60) | |
| | 5′(M)₀(F)₀(M)₆(F)₁(M)₁(F)₄(M)₉ 3′; |
| | j) |
| (SEQ ID NO: 61) | |
| | 5′(M)₀(F)₀(M)₀(F)₀(M)₈(F)₄(M)₉ 3′; |
| | k) |
| (SEQ ID NO: 62) | |
| | 5′(M)₀(F)₀(M)₀(F)₀(M)₆(F)₄(M)₁₀ 3′; |
| | l) |
| (SEQ ID NO: 63) | |
| | 5′(M)₀(F)₀(M)₅(F)₁(M)₁(F)₄(M)₉ 3′; |
| | m) |
| (SEQ ID NO: 64) | |
| | 5′(M)₀(F)₀(M)₀(F)₀(M)₇(F)₄(M)₉ 3′; |
| | n) |
| (SEQ ID NO: 65) | |
| | 5′(M)₀(F)₀(M)₀(F)₀(M)₇(F)₅(M)₉ 3′; |
| | and |
| | o) |
| (SEQ ID NO: 66) | |
| | 5′(M)₀(F)₀(M)₀(F)₀(M)₆(F)₅(M)₉ 3′. |

Embodiment 74. The isolated oligonucleotide of any one of embodiments 40-45, wherein in the antisense strand at most five nucleotides are modified with the third modification at positions 2, 3, 6, 14 and 16, from the nucleotide complementary to the first nucleotide at the 5′-terminus of the antisense strand.
Embodiment 75. The isolated oligonucleotide of any one of embodiments 40-45, wherein in the antisense strand at most five nucleotides are modified with the third modification at positions 2, 6, 7, 14 and 16, from the nucleotide complementary to the first nucleotide at the 5′-terminus of the antisense strand.
Embodiment 76. The isolated oligonucleotide of any one of embodiments 40-45, wherein in the antisense strand at most six nucleotides are modified with the third modification at positions 2, 3, 6, 8, 9 and 16, from the nucleotide complementary to the first nucleotide at the 5′-terminus of the antisense strand.
Embodiment 77. The isolated oligonucleotide of any one of embodiments 40-45, wherein in the antisense strand at most six nucleotides are modified with the third modification at positions 2, 3, 5, 8, 9 and 16, from the nucleotide complementary to the first nucleotide at the 5′-terminus of the antisense strand.
Embodiment 78. The isolated oligonucleotide of any one of embodiments 40-45, wherein in the antisense strand at most seven nucleotides are modified with the third modification at positions 2, 3, 5, 7, 9, 14 and 16, from the nucleotide complementary to the first nucleotide at the 5′-terminus of the antisense strand.
Embodiment 79. The isolated oligonucleotide of any one of embodiments 40-45, wherein in the antisense strand at most seven nucleotides are modified with the third modification at positions 2, 3, 5, 7, 10, 14 and 16, from the nucleotide complementary to the first nucleotide at the 5′-terminus of the antisense strand.
Embodiment 80. The isolated oligonucleotide of any one of embodiments 1-79, wherein X₁ is 18-21 and X2 is 20-23.
Embodiment 81. The isolated oligonucleotide of embodiment 80, wherein X₁ is 20 or 21 and X2 is 22 or 23.
Embodiment 82. The isolated oligonucleotide of any one of embodiments 1-80, wherein X2 equals X₁ plus 2.
Embodiment 83. The isolated oligonucleotide of any one of embodiments 1-82, wherein the sense strand comprises at least one nucleotide having a modified phosphate backbone.
Embodiment 84. The isolated oligonucleotide of any one of embodiments 1-83, wherein the antisense strand comprises at least one nucleotide having a modified phosphate backbone.
Embodiment 85. The isolated oligonucleotide of embodiment 83 or 84, wherein the modified phosphate backbone comprises a modified phosphodiester bond.
Embodiment 86. The isolated oligonucleotide of embodiment 85, wherein the modified phosphodiester bond is modified by replacing one or more oxygen atoms with a moiety, wherein the moiety is bonded to the phosphorus atom in the phosphodiester bond with a carbon, nitrogen, or sulfur atom in the moiety, or by forming a 2′-5′ linkage.
Embodiment 87. The isolated oligonucleotide of embodiment 85, wherein the modified phosphodiester bond comprises phosphorothioate, phosphorodithioate, methylphosphonate, phosphoramidate diester, mesyl phosphoramidate, or phosphonoacetate.
Embodiment 88. The isolated oligonucleotide of any one of embodiments 1-84, comprising one or more non-natural base-containing nucleotide, a locked nucleotide, or an abasic nucleotide.
Embodiment 89. The isolated oligonucleotide of any one of embodiments 1-88, wherein the terminal nucleotide at the 5′ end comprises a phosphate mimic.
Embodiment 90. The isolated oligonucleotide of embodiment 89, wherein the 5′-phosphate mimic is ethylphosphonate, vinylphosphonate, or an analog thereof
Embodiment 91. The isolated oligonucleotide of any one of embodiments 1-90, wherein the antisense strand comprises at least two single-stranded nucleotides at the 3′-terminus.
Embodiment 92. The isolated oligonucleotide of any one of embodiments 1-91, wherein in the sense strand or the antisense strand or both, a terminal or internal nucleotide is linked to a targeting ligand.
Embodiment 93. The isolated oligonucleotide of embodiment 92, wherein the targeting ligand is selected from one or more of a carbohydrate, a peptide, a lipid, an antibody or a fragment thereof, an aptamer, an albumin, a fibrinogen, and a folate.
Embodiment 94. The isolated oligonucleotide of embodiment 93, wherein the targeting ligand is GalNAc.
Embodiment 95. The isolated oligonucleotide of any one of embodiments 1-94, wherein the antisense strand is complementary to an mRNA, wherein the sequence-specific hybridization of the antisense strand to the mRNA results in degradation of the mRNA.
Embodiment 96. The isolated oligonucleotides of any one of embodiments 1-95, wherein they can target any specific gene target, including but not limited to PCSK9, ANGPTL-3, AGT, HSD17b3, C3, CFB, and HBV.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. An isolated oligonucleotide, comprising:
(a) a sense strand comprising X₁ nucleotides, wherein at least one nucleotide is modified with a first modification,
each of the remaining nucleotides is independently modified with a second modification, and
X₁ is an integer selected from 13-36,
wherein the first modification and the second modification are different; and
(b) an antisense strand comprising X₂ nucleotides, wherein at least one nucleotide is modified with a third modification,
each of the remaining nucleotides is independently modified with a fourth modification, and
X₂ is an integer selected from 18-31,
wherein the third modification and the fourth modification are different.

2. The isolated oligonucleotide of claim 1, wherein the first modification is modification of the sugar moiety of the at least one nucleotide at the 2′-position selected from 2′-F modification, 2′-CN modification, 2′-N3 modification, 2′-deoxy modification, and an equivalent thereof, and a combination thereof,
preferably wherein the first modification is 2′-F modification, 2′-CN modification, 2′-N3 modification, or 2′-deoxy modification, or a stereoisomer thereof,
more preferably wherein the first modification is 2′-F modification, 2′-CN modification, or 2′-N3 modification, or a stereoisomer thereof,
even more preferably wherein the first modification is 2′-F modification or a stereoisomer thereof.

3. The isolated oligonucleotide of claim 1, wherein the second modification is modification of the sugar moiety of one or more of the remaining nucleotides at the 2′-position selected from 2′-C₁-C₆ alkyl, 2′-OR modification wherein R is C₁-C₆ alkyl optionally substituted with C₁-C₆ alkoxy, acetamide, phenyl, or heteroaryl comprising a 5- or 6-membered ring and 1 or 2 heteroatoms selected from N, O, and S, 2′-amino, and morpholino replacement, and an equivalent thereof, and a combination thereof,
preferably wherein the second modification is 2′-OR modification, more preferably wherein the second modification is 2′-O-methyl modification or 2′-methoxyethoxy modification,
even more preferably wherein the second modification is 2′-O-methyl modification.

4. The isolated oligonucleotide of claim 1, wherein the first modification is 2′-F modification or a stereoisomer thereof, and the second modification is 2′-O-methyl modification or 2′-methoxyethoxy modification,
preferably wherein the first modification is 2′-F modification or a stereoisomer thereof, and the second modification is 2′-O-methyl modification.

5. The isolated oligonucleotide of claim 1, wherein the third modification is modification of the sugar moiety of the at least one nucleotide at the 2′-position selected from 2′-F modification, 2′-CN modification, 2′-N3 modification, 2′-deoxy modification, and an equivalent thereof, and a combination thereof,
preferably wherein the third modification is 2′-F modification, 2′-CN modification, 2′-N3 modification, or 2′-deoxy modification, or a stereoisomer thereof,
more preferably wherein the third modification is 2′-F modification, 2′-CN modification, or 2′-N3 modification, or a stereoisomer thereof,
even more preferably wherein the third modification is 2′-F modification or a stereoisomer thereof.

6. The isolated oligonucleotide of claim 1, wherein the fourth modification is modification of the sugar moiety of one or more of the remaining nucleotides at the 2′-position selected from 2′-C₁-C₆ alkyl, 2′-OR modification wherein R is C₁-C₆ alkyl optionally substituted with C₁-C₆ alkoxy, acetamide, phenyl, or heteroaryl comprising a 5- or 6-membered ring and 1 or 2 heteroatoms selected from N, O, and S, 2′-amino, and morpholino replacement, and an equivalent thereof, and a combination thereof,
preferably wherein the fourth modification is 2′-OR modification,
more preferably wherein the fourth modification is 2′-O-methyl modification or 2′-methoxyethoxy modification,
even more preferably wherein the fourth modification is 2′-O-methyl modification.

7. The isolated oligonucleotide of claim 1, wherein the third modification is 2′-F modification or a stereoisomer thereof, and the fourth modification is 2′-O-methyl modification or 2′-methoxyethoxy modification,
preferably wherein the third modification is 2′-F modification or a stereoisomer thereof, and the fourth modification is 2′-O-methyl modification.

8. The isolated oligonucleotide of claim 1, wherein in the sense strand at least three nucleotides are modified with the first modification, optionally wherein at least two of the at least three nucleotides modified with the first modification are consecutively located, or optionally wherein at least three of the at least three nucleotides modified with the first modification are consecutively located.

9. The isolated oligonucleotide of claim 1, wherein in the sense strand at least four nucleotides are modified with the first modification, optionally wherein at least three of the at least four nucleotides modified with the first modification are consecutively located, or optionally wherein at least four of the at least four nucleotides modified with the first modification are consecutively located.

10. The isolated oligonucleotide of claim 1, wherein in the sense strand at least five nucleotides are modified with the first modification, optionally wherein at least three of the at least five nucleotides modified with the first modification are consecutively located, or optionally wherein at least four of the at least five nucleotides modified with the first modification are consecutively located.

11. The isolated oligonucleotide of claim 8, wherein the at least three nucleotides, the at least four nucleotides, or the at least five nucleotides modified with the first modification are located from position 10 to position 15 from the nucleotide complementary to the first nucleotide at the 5′-terminus of the antisense strand,
optionally wherein two of the at least three nucleotides modified with the first modification are located at positions selected from position 10, 11, 12, and 13 from the nucleotide complementary to the first nucleotide at the 5′-terminus of the antisense strand, or
optionally wherein three of the at least three nucleotides modified with the first modification are located at positions selected from position 10, 11, 12, and 13 from the nucleotide complementary to the first nucleotide at the 5′-terminus of the antisense strand, or
optionally wherein one of the at least three nucleotides modified with the first modification is located at position 11 from the nucleotide complementary to the first nucleotide at the 5′-terminus of the antisense strand.

12. The isolated oligonucleotide of claim 8, wherein one of the at least four nucleotides modified with the first modification is located at position 10 from the nucleotide complementary to the first nucleotide at the 5′-terminus of the antisense strand, or
optionally wherein one of the at least four nucleotides modified with the first modification is located at position 14 from the nucleotide complementary to the first nucleotide at the 5′-terminus of the antisense strand, or
optionally wherein two of the at least four nucleotides modified with the first modification are located from position 10 to position 14 from the nucleotide complementary to the first nucleotide at the 5′-terminus of the antisense strand.

13. The isolated oligonucleotide of claim 8, wherein not all of the at least three nucleotides modified with the first modification are consecutively located.

14. The isolated oligonucleotide of claim 8, wherein the at least three nucleotides, the at least four nucleotides, or the at least five nucleotides are modified with 2′-F modification.

15. The isolated oligonucleotide of claim 8, wherein in the sense strand
at most three nucleotides are modified with the first modification at positions 12, 13 and 15, from the nucleotide complementary to the first nucleotide at the 5′-terminus of the antisense strand,
at most four nucleotides are modified with the first modification at positions 11, 12, 13 and 14, from the nucleotide complementary to the first nucleotide at the 5′-terminus of the antisense strand,
at most four nucleotides are modified with the first modification at positions 10, 11, 12 and 13, from the nucleotide complementary to the first nucleotide at the 5′-terminus of the antisense strand,
at most four nucleotides are modified with the first modification at positions 10, 12, 13 and 15, from the nucleotide complementary to the first nucleotide at the 5′-terminus of the antisense strand,
at most five nucleotides are modified with the first modification at positions 10, 11, 12, 13 and 15, from the nucleotide complementary to the first nucleotide at the 5′-terminus of the antisense strand, or
at most five nucleotides are modified with the first modification at positions 10, 11, 12, 13 and 14, from the nucleotide complementary to the first nucleotide at the 5′-terminus of the antisense strand.

16. The isolated oligonucleotide of claim 1, wherein the sense strand comprises nucleotides modified with 2′-F modification, and nucleotides modified with 2′-O-methyl modification, according to the formula:
5′ (M)_{g}(F)_{f}(M)ₑ(F)_{d}(M)_{c}(F)_{b}(M)ₐ 3′,
wherein M is 2′-O-methyl modified nucleotide, F is 2′-F modified nucleotide, and each of a, b, c, d, e, f, and g are any one of 0-16, and indicates the number of consecutive nucleotides modified with the modification, and wherein the sense strand is any one of:

| | a) |
|---|---|
| (SEQ ID NO: 52) | |
| | 5′ (M)₀(F)₀(M)₀(F)₀(M)₈(F)₃(M)₁₀ 3′; |
| | b) |
| (SEQ ID NO: 53) | |
| | 5′ (M)₀(F)₀(M)₅(F)₁(M)₁(F)₃(M)₁₀ 3′; |
| | c) |
| (SEQ ID NO: 54) | |
| | 5′ (M)₀(F)₀(M)₀(F)₀(M)₇(F)₃(M)₁₀ 3′; |
| | d) |
| (SEQ ID NO: 55) | |
| | 5′ (M)₀(F)₀(M)₅(F)₁(M)₁(F)₂(M)₁₁ 3′; |
| | e) |
| (SEQ ID NO: 56) | |
| | 5′ (M)₀(F)₀(M)₆(F)₁(M)₁(F)₂(M)₁₁ 3′; |
| | f) |
| (SEQ ID NO: 57) | |
| | 5′ (M)₆(F)₁(M)₁(F)₂(M)₁(F)₁(M)₉ 3′; |
| | g) |
| (SEQ ID NO: 58) | |
| | 5′ (M)₅(F)₁(M)₁(F)₂(M)₁(F)₁(M)₉ 3′; |
| | h) |
| (SEQ ID NO: 59) | |
| | 5′ (M)₀(F)₀(M)₀(F)₀(M)₇(F)₄(M)₁₀ 3′; |
| | i) |
| (SEQ ID NO: 60) | |
| | 5′ (M)₀(F)₀(M)₆(F)₁(M)₁(F)₄(M)₉ 3′; |
| | j) |
| (SEQ ID NO: 61) | |
| | 5′ (M)₀(F)₀(M)₀(F)₀(M)₈(F)₄(M)₉ 3′; |
| | k) |
| (SEQ ID NO: 62) | |
| | 5′ (M)₀(F)₀(M)₀(F)₀(M)₆(F)₄(M)₁₀ 3′; |
| | l) |
| (SEQ ID NO: 63) | |
| | 5′ (M)₀(F)₀(M)₅(F)₁(M)₁(F)₄(M)₉ 3′; |
| | m) |
| (SEQ ID NO: 64) | |
| | 5′ (M)₀(F)₀(M)₀(F)₀(M)₇(F)₄(M)₉ 3′; |
| | n) |
| (SEQ ID NO: 65) | |
| | 5′ (M)₀(F)₀(M)₀(F)₀(M)₇(F)₅(M)₉ 3′; |
| | and |
| | o) |
| (SEQ ID NO: 66) | |
| | 5′ (M)₀(F)₀(M)₀(F)₀(M)₆(F)₅(M)₉ 3′. |

17. The isolated oligonucleotide of claim 1, wherein in the antisense strand at most seven nucleotides are modified with the third modification,
optionally wherein at most four of the at most seven nucleotides modified with the third modification are located from position 2 to position 8 from the first nucleotide at the 5′-terminus of the antisense strand, or
optionally wherein at most two of the at most seven nucleotides modified with the third modification are consecutively located, or
optionally wherein three or four of the at most seven nucleotides modified with the third modification are located at positions selected from position 2, 3, 5, 6, 7, and 8 from the first nucleotide at the 5′-terminus of the antisense strand, or
optionally wherein at least one of the at most seven nucleotides modified with the third modification is located at position 14 from the first nucleotide at the 5′-terminus of the antisense strand, or
optionally wherein two or three of the at most seven nucleotides modified with the third modification are located at positions selected from position 2, 3, 5, and 6 from the first nucleotide at the 5′-terminus of the antisense strand, or
optionally wherein three of the at most seven nucleotides modified with the third modification are located at positions selected from position 2, 3, 5, and 6 from the first nucleotide at the 5′-terminus of the antisense strand, or
optionally wherein two of the at most seven nucleotides modified with the third modification are located at positions 8 and 14 from the nucleotide complementary to the first nucleotide at the 5′-terminus of the antisense strand, or
optionally wherein one or two of the at most seven nucleotides modified with the third modification are located at positions selected from position 14 and 16 from the first nucleotide at the 5′-terminus of the antisense strand, or
optionally wherein two of the at most seven nucleotides modified with the third modification are located at positions 14 and 16 from the first nucleotide at the 5′-terminus of the antisense strand.

18. The isolated oligonucleotide of claim 17, wherein at most six nucleotides are modified with the third modification.

19. The isolated oligonucleotide of claim 17, wherein at most five nucleotides are modified with the third modification.

20. The isolated oligonucleotide of claim 17, wherein at most four nucleotides are modified with the third modification.

21. The isolated oligonucleotide of claim 17, wherein at most three nucleotides are modified with the third modification.

22. The isolated oligonucleotide of claim 17, wherein the at least seven nucleotides, at least six nucleotides, at least five nucleotides, at least four nucleotides, or at least three nucleotides are modified with 2′-F modification,

23. The isolated oligonucleotide of claim 17, wherein in the antisense strand:
at most five nucleotides are modified with the third modification at positions 2, 3, 6, 14 and 16, from the nucleotide complementary to the first nucleotide at the 5′-terminus of the antisense strand, or
at most five nucleotides are modified with the third modification at positions 2, 6, 7, 14 and 16, from the nucleotide complementary to the first nucleotide at the 5′-terminus of the antisense strand.

24. The isolated oligonucleotide of claim 17, wherein in the antisense strand:
at most six nucleotides are modified with the third modification at positions 2, 3, 6, 8, 9 and 16, from the nucleotide complementary to the first nucleotide at the 5′-terminus of the antisense strand, or
at most six nucleotides are modified with the third modification at positions 2, 3, 5, 8, 9 and 16, from the nucleotide complementary to the first nucleotide at the 5′-terminus of the antisense strand.

25. The isolated oligonucleotide of claim 17, wherein in the antisense strand
at most seven nucleotides are modified with the third modification at positions 2, 3, 5, 7, 9, 14 and 16, from the nucleotide complementary to the first nucleotide at the 5′-terminus of the antisense strand, or
at most seven nucleotides are modified with the third modification at positions 2, 3, 5, 7, 10, 14 and 16, from the nucleotide complementary to the first nucleotide at the 5′-terminus of the antisense strand.

26. The isolated oligonucleotide of claim 1, wherein the antisense strand comprises nucleotides modified with 2′-F modification, and nucleotides modified with 2′-O-methyl modification, according to the formula:
3′ (M)ₐ(F)_{b}(M)_{c}(F)_{d}(M)ₑ(F)_{f}(M)_{g}(F)ₕ(M)ᵢ(F)ⱼ(M)ₖ
(F)ₗ(M)ₘ(F)ₙ(M)ₒ 5′,
wherein M is 2′-O-methyl modified nucleotide, F is 2′-F modified nucleotide, and each of a, b, c, d, e, f, g, h, i, j, k, l, m, n, and o are any one of 0-16, and indicates the number of consecutive nucleotides modified with the modification, wherein the antisense strand is any one of:
1)
(SEQ ID NO: 14)
3′(M)₀(F)₀(M)₀(F)₀(M)₀(F)₀(M)₇(F)₁(M)₁(F)₁(M)₇(F)₂(M)₂(F)₁(M)₁ 5′;
2)
(SEQ ID NO: 15)
3′(M)₀(F)₀(M)₀(F)₀(M)₀(F)₀(M)₇(F)₁(M)₁(F)₁(M)₇(F)₁(M)₂(F)₂(M)₁ 5′;
3)
(SEQ ID NO: 16)
3′(M)₀(F)₀(M)₀(F)₀(M)₀(F)₀(M)₇(F)₁(M)₁(F)₁(M)₆(F)₂(M)₃(F)₁(M)₁ 5′;
4)
(SEQ ID NO: 17)
3′(M)₀(F)₀(M)₀(F)₀(M)₇(F)₁(M)₁(F)₁(M)₅(F)₁(M)₁(F)₁(M)₃(F)₁(M)₁ 5′;
5)
(SEQ ID NO: 18)
3′(M)₀(F)₀(M)₀(F)₀(M)₇(F)₁(M)₁(F)₁(M)₄(F)₁(M)₂(F)₁(M)₃(F)₁(M)₁ 5′;
6)
(SEQ ID NO: 20)
3′(M)₀(F)₀(M)₀(F)₀(M)₀(F)₀(M)₇(F)₁(M)₁(F)₁(M)_{:}(F)₁(M)₂(F)₁(M)₁ 5′;
7)
(SEQ ID NO: 21)
3′(M)₀(F)₀(M)₀(F)₀(M)₀(F)₀(M)₀(F)₀(M)₉(F)₁(M)₇(F)₁(M)₃(F)₁(M)₁ 5′;
8)
(SEQ ID NO: 22)
3′(M)₀(F)₀(M)₀(F)₀(M)₇(F)₁(M)₁(F)₁(M)₄(F)₂(M)₁(F)₁(M)₂(F)₂(M)₁ 5′;
9)
(SEQ ID NO: 23)
3′(M)₀(F)₀(M)₀(F)₀(M)₇(F)₁(M)₁(F)₁(M)₄(F)₂(M)₂(F)₁(M)₁(F)₂(M)₁ 5′;
10)
(SEQ ID NO: 24)
3′(M)₀(F)₀(M)₀(F)₀(M)₇(F)₁(M)₁(F)₁(M)₄(F)₁(M)₁(F)₁(M)₁(F)₂(M)₁ 5′;
11)
(SEQ ID NO: 25)
3′(M)₀(F)₀(M)₀(F)₀(M)₇(F)₁(M)₁(F)₁(M)₃(F)₁(M)₂(F)₁(M)₁(F)₁(M)₁(F)₂(M)₁ 5′;
12)
(SEQ ID NO: 27)
3′(M)₀(F)₀(M)₄(F)₁(M)₁(F)₁(M)₁(F)₁(M)₃(F)₁(M)₁(F)₁(M)₁(F)₃(M)₁(F)₁(M)₁ 5′;
13)
(SEQ ID NO: 30)
3′(M)₀(F)₀(M)₀(F)₀(M)₀(F)₀(M)₆(F)₁(M)₁(F)₁(M)₇(F)₁(M)₃(F)₁(M)₁ 5′;
14)
(SEQ ID NO: 50)
3′(M)₀(F)₀(M)₀(F)₀(M)₀(F)₀(M)₆(F)₁(M)₁(F)₁(M)₇(F)₂(M)₂(F)₁(M)₁ 5′;
15)
(SEQ ID NO: 45)
3′(M)₀(F)₀(M)₀(F)₀(M)₀(F)₀(M)₆(F)₁(M)₁(F)₁(M)₇(F)₁(M)₂(F)₂(M)₁ 5′;
16)
(SEQ ID NO: 46)
3′(M)₀(F)₀(M)₀(F)₀(M)₀(F)₀(M)₆(F)₁(M)₁(F)₁(M)₆(F)₂(M)₃(F)₁(M)₁ 5′;
17)
(SEQ ID NO: 47)
3′(M)₀(F)₀(M)₀(F)₀(M)₆(F)₁(M)₁(F)₁(M)₅(F)₁(M)₁(F)₁(M)₃(F)₁(M)₁ 5′;
18)
(SEQ ID NO: 48)
3′(M)₀(F)₀(M)₀(F)₀(M)₆(F)₁(M)₁(F)₁(M)₄(F)₁(M)₂(F)₁(M)₃(F)₁(M)₁ 5′;
19)
(SEQ ID NO: 49)
3′(M)₀(F)₀(M)₀(F)₀(M)₆(F)₁(M)₁(F)₁(M)₄(F)₂(M)₁(F)₁(M)₃(F)₁(M)₁ 5′;
20)
(SEQ ID NO: 37)
3′(M)₀(F)₀(M)₀(F)₀(M)₀(F)₀(M)₆(F)₁(M)₁(F)₁(M)₈(F)₁(M)₂(F)₁(M)₁ 5′;
21)
(SEQ ID NO: 38)
3′(M)₀(F)₀(M)₀(F)₀(M)₀(F)₀(M)₀(F)₀(M)₈(F)₁(M)₇(F)₁(M)₃(F)₁(M)₁ 5′;
22)
(SEQ ID NO: 39)
3′(M)₀(F)₀(M)₀(F)₀(M)₆(F)₁(M)₁(F)₁(M)₄(F)₂(M)₁(F)₁(M)₂(F)₂(M)₁ 5′;
23)
(SEQ ID NO: 40)
3′(M)₀(F)₀(M)₀(F)₀(M)₆(F)₁(M)₁(F)₁(M)₄(F)₂(M)₂(F)₁(M)₁(F)₂(M)₁ 5′;
24)
(SEQ ID NO: 41)
3′(M)₀(F)₀(M)₆(F)₁(M)₁(F)₁(M)₄(F)₁(M)₁(F)₁(M)₁(F)₁(M)₁(F)₂(M)₁ 5′;
and
25)
(SEQ ID NO: 42)
3′(M)₀(F)₀(M)₆(F)₁(M)₁(F)₁(M)₃(F)₁(M)₂(F)₁(M)₁(F)₁(M)₁(F)₂(M)₁ 5′.

27. The isolated oligonucleotide of claim 26, wherein the sense strand comprises nucleotides modified with 2′-F modification, and nucleotides modified with 2′-O-methyl modification, according to the formula:
5′ (M)_{g}(F)_{f}(M)ₑ(F)_{d}(M)_{c}(F)_{b}(M)ₐ 3′,
wherein M is 2′-O-methyl modified nucleotide, F is 2′-F modified nucleotide, and each of a, b, c, d, e, f, and g are any one of 0-16, and indicates the number of consecutive nucleotides modified with the modification, and wherein the sense strand is any one of:

| | a) |
|---|---|
| (SEQ ID NO: 52) | |
| | 5′(M)₀(F)₀(M)₀(F)₀(M)₈(F)₃(M)₁₀ 3′; |
| | b) |
| (SEQ ID NO: 53) | |
| | 5′(M)₀(F)₀(M)₅(F)₁(M)₁(F)₃(M)₁₀ 3′; |
| | c) |
| (SEQ ID NO: 54) | |
| | 5′(M)₀(F)₀(M)₀(F)₀(M)₇(F)₃(M)₁₀ 3′; |
| | d) |
| (SEQ ID NO: 55) | |
| | 5′(M)₀(F)₀(M)₅(F)₁(M)₁(F)₂(M)₁₁ 3′; |
| | e) |
| (SEQ ID NO: 56) | |
| | 5′(M)₀(F)₀(M)₆(F)₁(M)₁(F)₂(M)₁₁ 3′; |
| | f) |
| (SEQ ID NO: 57) | |
| | 5′(M)₆(F)₁(M)₁(F)₂(M)₁(F)₁(M)₉ 3′; |
| | g) |
| (SEQ ID NO: 58) | |
| | 5′(M)₅(F)₁(M)₁(F)₂(M)₁(F)₁(M)₉ 3′; |
| | h) |
| (SEQ ID NO: 59) | |
| | 5′(M)₀(F)₀(M)₀(F)₀(M)₇(F)₄(M)₁₀ 3′; |
| | i) |
| (SEQ ID NO: 60) | |
| | 5′(M)₀(F)₀(M)₆(F)₁(M)₁(F)₄(M)₉ 3′; |
| | j) |
| (SEQ ID NO: 61) | |
| | 5′(M)₀(F)₀(M)₀(F)₀(M)₈(F)₄(M)₉ 3′; |
| | k) |
| (SEQ ID NO: 62) | |
| | 5′(M)₀(F)₀(M)₀(F)₀(M)₆(F)₄(M)₁₀ 3′; |
| | l) |
| (SEQ ID NO: 63) | |
| | 5′(M)₀(F)₀(M)₅(F)₁(M)₁(F)₄(M)₉ 3′; |
| | m) |
| (SEQ ID NO: 64) | |
| | 5′(M)₀(F)₀(M)₀(F)₀(M)₇(F)₄(M)₉ 3′; |
| | n) |
| (SEQ ID NO: 65) | |
| | 5′(M)₀(F)₀(M)₀(F)₀(M)₇(F)₅(M)₉ 3′; |
| | and |
| | o) |
| (SEQ ID NO: 66) | |
| | 5′(M)₀(F)₀(M)₀(F)₀(M)₆(F)₅(M)₉ 3′. |

28. The isolated oligonucleotide of claim 15, wherein in the antisense strand:
at most five nucleotides are modified with the third modification at positions 2, 3, 6, 14 and 16, from the nucleotide complementary to the first nucleotide at the 5′-terminus of the antisense strand,
at most five nucleotides are modified with the third modification at positions 2, 6, 7, 14 and 16, from the nucleotide complementary to the first nucleotide at the 5′-terminus of the antisense strand,
at most six nucleotides are modified with the third modification at positions 2, 3, 6, 8, 9 and 16, from the nucleotide complementary to the first nucleotide at the 5′-terminus of the antisense strand,
at most six nucleotides are modified with the third modification at positions 2, 3, 5, 8, 9 and 16, from the nucleotide complementary to the first nucleotide at the 5′-terminus of the antisense strand,
at most seven nucleotides are modified with the third modification at positions 2, 3, 5, 7, 9, 14 and 16, from the nucleotide complementary to the first nucleotide at the 5′-terminus of the antisense strand, or
at most seven nucleotides are modified with the third modification at positions 2, 3, 5, 7, 10, 14 and 16, from the nucleotide complementary to the first nucleotide at the 5′-terminus of the antisense strand.

29. The isolated oligonucleotide of claim 1, wherein the sense strand comprises at least one nucleotide having a modified phosphate backbone, and/or the antisense strand comprises at least one nucleotide having a modified phosphate backbone.

30. The isolated oligonucleotide of claim 1, comprising one or more non-natural base-containing nucleotide, a locked nucleotide, or an abasic nucleotide.

31. The isolated oligonucleotide of claim 1, wherein the terminal nucleotide at the 5′ end comprises a phosphate mimic.

32. The isolated oligonucleotide of claim 1, wherein in the sense strand or the antisense strand or both, a terminal or internal nucleotide is linked to a targeting ligand,
optionally wherein the targeting ligand is selected from one or more of a carbohydrate, a peptide, a lipid, an antibody or a fragment thereof, an aptamer, an albumin, a fibrinogen, and a folate, or
optionally wherein the targeting ligand is GalNAc.
